(19) United States
(12) Patent Application Publication
(10) Pub. No.: US 2006/0160087 A1
(43) Pub. Date: Jul. 20, 2006

(54) Monitoring and treatment of amyotrophic lateral sclerosis

(76) Inventors: Michael McGrath, Burlingame, CA (US); Kenneth G. Hadlock, San Francisco, CA (US)

Correspondence Address: FOLEY AND LARDNER LLP;SUITE 500, 3000 K STREET NW, WASHINGTON, DC, 20007, US

(21) Appl. No.: 10/544,059

(22) PCT Filed: Jan. 30, 2004
(86) PCT No.: PCT/US04/02704

§ 371 Date: Mar. 10, 2006

(30) Foreign Application Priority Data
Jan. 31, 2003 (US) 60444224

(51) Int. Cl. C12Q 1/68; G01N 33/567

(52) U.S. Cl. 435/6; 435/7.2

(57) ABSTRACT

The invention provides methods of monitoring amyotrophic lateral sclerosis (ALS) disease development or progression and monitoring an ALS therapy in an individual by determining the presence or absence of Herv-K/HML-2 expression in a biological sample from the individual. The invention is also directed to methods for aiding diagnosis of ALS by determining expression of Herv-K/HML-2 in a biological sample from the individual. The invention is also directed to methods of reducing Herv-K/HML-2 expression in infected cells and individuals. The invention includes reagents for use in these methods.

### TECHNICAL FIELD

The invention relates to the fields of Amyotrophic Lateral Sclerosis (ALS) disease and endogenous retroviruses. More specifically, it pertains to the expression of a specific endogenous retrovirus in individuals with ALS and monitoring of ALS progression, monitoring ALS therapy and treating patients with ALS.

### BACKGROUND OF THE INVENTION

Amyotrophic lateral sclerosis (ALS), known colloquially as Lou Gehrig's disease, is a heterogeneous group of progressive neurodegenerative disorders characterized by a selective loss of upper and/or lower motor neurons in the brain and spinal cord. Affected individuals demonstrate a variety of symptoms including twitching and cramping of muscles, loss of motor control in hands and arms, impaired use of the arms and legs, weakness and fatigue, tripping and falling, dropping things, slurred or thick speech and difficulty breathing or swallowing. Most cases of ALS are sporadic, however, 5-10% are familial. ALS eventually results in death of the affected individual, typically within one to five years of symptom onset.

Clinically, ALS is typically characterized by progressive muscle weakness, wasting and fasiculations (i.e., cramping), in conjunction with spasticity, hyperreflexia and pathological corticospinal tract findings. Generally, ALS is neuropathologically characterized by degeneration of motor neurons in the brainstem, spinal cord and cerebral cortex. ALS tissue is also characterized by neuroinflammatory changes that are typical of several neurodegenerative conditions (McGeer et al. (2002) Muscle Neive 26:459-470). These neuroinflammatory changes are seen in sporadic and familial ALS and in the superoxide dismutase 1 (SOD1) transgenic mouse model for ALS.

Immune dysfunction has also been proposed to be involved with ALS. Helper and cytotoxic T lymphocytes expressing the major histocompatibility glycoproteins HLA-A, B, C and HLA-DR were found in ALS spinal cord (McGeer et al. (1991) Can J. Neurol. Sci. 18:376-379). Cellular infiltrates consisting mainly of T lymphocytes and macrophages were found in muscle biopsy specimens from autopsied ALS patients (Troost et al. (1992) Clin. Neuropathol. 11:115-120). Most of the T lymphocytes and macrophages surrounding the atrophied muscle fibers expressed a high level of HLA-DR indicating an activated state of the cells and suggesting a role for the cells in ALS-associated muscle atrophy. Also, Schwann cells expressing HLA-DR have been identified in the endoneurium of peripheral nerve in ALS (Olivera et al. (1994) Arq. Neuropsiquiatr. 52:493-500).

In addition, both familial and sporadic ALS are characterized by high levels of immune activation of the microglial cells of the spinal cord and cerebellum, with large numbers of reactive microglial and astrocytes found particularly throughout the degenerating areas (McGeer et al. (2002)). Other clinical observations with ALS include the presence of significant deposits of endogenous IgG and spheroid bodies, which are composed of various classes of neurofilament proteins (McGeer et al. (2002), Kawamata et al. (1992) Am. J. Pathol. 140:691-707, Alexianu et al. (2001) Neurology 57:1282-1289). Use of the SOD1 mouse model has confirmed that immune activation of the microglial cells proceeded overt hind limb paralysis and increased as paralysis increased (Alexianu et al. (2001)).

Accordingly, immune activation of cells in and around the spinal cord, including microglial and lymphocytes, appears to play a role in neuroinflammation and neurodegeneration in ALS.

ALS is diagnosed using a variety of tests and examinations, including muscle and nerve biopsy, spinal tap, X-rays, magnetic resonance imaging (MRI) and electrodiagnostic tests, many of which involve invasive procedures or complex imaging and analysis. There remains a need for additional measures of ALS disease progression for use in monitoring of the disease as well as in evaluation of potential therapies for ALS. There also remains a need for effective therapies for amelioration of symptoms of ALS.

All publications and patent applications cited herein are hereby incorporated by reference in their entirety.

### SUMMARY OF THE INVENTION

The present invention provides methods of monitoring development or progression of ALS in an individual comprising determining the presence or absence of Herv-K/HML-2 expression in a biological sample from the individual.

Accordingly, in one aspect of the invention, monitoring of ALS is done by comparing the level of Herv-K/HML-2 expression in a biological sample at different time points in the course of the disease, with the presence of Herv-K/HML-2 expression or an increase in the level of Herv-K/HML-2 expression generally being consistent with an increase in disease severity and/or rate of progression.

The present invention also provides methods of monitoring therapy of ALS in an individual comprising determining the presence, absence or level of Herv-K/HML-2 expression in a biological sample from the individual.

Accordingly, in another aspect of the invention, the effect of an ALS therapy is monitored by comparing Herv-K/HML-2 expression in a biological sample from the recipient of the therapy before and during treatment, with a decrease in expression of Herv-K/HML-2 generally being consistent with a positive effect of the therapy.

The present invention also provides methods for aiding diagnosis or prediction of ALS through detection of Herv-K/HML-2 expression in a biological sample from an individual. In some embodiments, detection of such Herv-K/HML-2 expression is combined with one or more other disease indicators for diagnosis of ALS. In some embodiments, detection of Herv-K/HML-2 expression in a biological sample from an individual may assist in classifying an ALS diagnosis.

The present invention also provides methods for ameliorating a symptom of ALS through decreasing Herv-K/HML-2 expression and/or suppressing Herv-K/HML-2 viral replication in the individual. The present invention also provides methods for ameliorating a symptom of ALS through reducing and/or suppressing the level of anti-Herv-K/HML-2 antibodies in an individual in need thereof.

The present invention also provides compositions comprising probes for Herv-K/HML-2 expression for use in the methods of the invention. Accordingly, in another aspect of the invention, probes specific for detecting Herv-K/HML-2 expression, particularly specific for detecting expression of Herv-K/HML-2 GAG expression are provided. The present invention also provides kits for use in monitoring ALS which comprise the probes specific for detecting Herv-K/HML-2 expression.

The present invention also provides pharmaceutical compositions comprising at least one Herv-K/HML-2 polypeptide or a polynucleotide that encodes a Herv-K/HML-2 polypeptide. In some embodiments, the pharmaceutical compositions comprise a Herv-K/HML-2 GAG polypeptide or a polynucleotide that encodes a Herv-K/HML-2 GAG polypeptide.

### BRIEF DESCRIPTION OF THE DRAWINGS

FIG. 1 is a schematic showing a map of the Herv-K/HML-2 provirus (bottom line) and of the major gene products encoded by an intact provirus (top line). The positions of the recombinant polypeptides described in Example 2 and Table 1 relative to the provirus and encoded products are also depicted.

FIG. 2A-2C lists the nucleotide and amino acid sequences for seven of the Herv-K/HML-2 GAG and ENV polynucleotides and polypeptides generated (KG-ME-2, SEQ ID NOs: 1 and 2; KG-PT-5, SEQ ID NOs: 3 and 4; KG-KQ-13, SEQ ID NOs: 5 and 6; KG-LH-24, SEQ ID NOs: 7 and 8; KE-WS-7, SEQ ID NOs: 9 and 10; KE-WS2-17, SEQ ID NOs: 11 and 12; KE-HKX-24, SEQ ID NOs: 13 and 14).

FIG. 3 is a graph depicting reactivity of purified SE-HA antigen with sera from ALS patients and non-ALS blood donors in a representative ELISA assay. The graph indicates the mean signal obtained (y axis) from individual plasmas from ALS patients (gray bars) or healthy donors (white bars). Results obtained from control wells and antibodies are also provided (black bars). Blank indicates wells without any added plasma or antibody. Anti 5H indicates results obtained with a monoclonal antibody to the sequence HHHHH, diluted 1:1000. All plasma was diluted to an IgG concentration of 100 μg/ml and tested in duplicate. Error bars indicate one standard deviation from the mean.

FIG. 4 is a schematic showing a map of the Herv-W provirus and of the major gene products encoded by an intact provirus. The map is based on a consensus sequence generated from multiple individual Herv-W sequences present in Genbank. The positions of the recombinant GAG and ENV polypeptides described in Example 4 relative to the provirus and encoded products are also depicted.

FIG. 5 is a graph depicting expression of multiple endogenous retroviruses upon monocyte activation. Total RNA from PBMCs from a healthy blood donor that attached to tissue culture flasks (black bars) or remained in suspension (white bars) was subjected to RT-PCR and the amplified products were hybridzed to 40 nucleotide long probes and bound probe was detected by with a luminescent substrate. The bars indicate the mean luminescent signal from triplicate wells after subtraction of background signal obtained in the absence of RT. The y axis is a logarithmic scale.

FIG. 6A is an alignment of amino acid sequences encoded by the indicated clones from the 5′ GAG region of HML-2, HML-1, HML4, HML-5 and HML-6. Dashes indicate gaps in the sequence. The shading indicates the degree of conservation with lighter shading indicating complete conservation for all 5 sequences and darker shading indicating lesser degrees of conservation. A consensus sequence showing the most common amino acids at a given position is presented at the bottom. FIG. 6B is a table indicating the percent amino acid identity between each of the 5 amino acid sequences in FIG. 6A.

FIG. 7 is a schematic showing a map of KG-ME-2 fragments used to localize the reactive epitope within KG-ME-2. The map indicates the amino acid sequences expressed by the various deletion constructs (names at the left) described in Example 6 and Table 1. The reactivity obtained with the constructs is indicated at right with the scale as follows: ++=highly reactive; −=non-reactive.

FIG. 8 is a graph depicting Herv-K RNA levels in PBMCs from patients with ALS or AD. The threshold cycles obtained with each of the samples and the various primers were employed to compare the Herv-K RNA levels to the actin expression of the same sample (y axis, logarithmic scale). The Herv-K expression levels of SE-HA reactive ALS patients (squares), SE-HA negative ALS patients (triangles), and AD patients (open circles) are indicated and the black lines indicate the median value of each of the three groups. The significance levels of the differences between the median values as determined by the Mann-Whitney test are indicated above the graph.

### MODES FOR CARRYING OUT THE INVENTION

We have discovered that a high percentage of individuals with sporadic ALS have serum antibodies reactive to GAG and/or ENV proteins of the endogenous retrovirus Herv-K/HML-2. The percentage of individuals with this immunoreactivity was significantly higher in those with ALS than in non-ALS blood donors. We have also observed that the presence of antibodies reactive to particular Herv-K/HML-2 GAG proteins is concurrent with the incidence of neurological symptoms in the ALS individuals. The presence of anti-Herv-K/HML-2 antibodies indicates that Herv-K/HML-2 genes have been and/or are being expressed in the individual.

Thus, we have discovered methods for monitoring ALS disease progression and/or activity, methods for monitoring effectiveness of agents for the treatment of ALS, as well as methods for aiding diagnosis of ALS disease based on expression of Herv-K/HML-2 in an individual. Our discovery also indicates a potentially significant target for therapeutic intervention, as the expression of Herv-K/HML-2 in these individuals may mediate at least one symptom of the disease.

Since retroviral GAG proteins generally require full-length retroviral RNA in order to be produced, the existence of anti-Herv-K/HML-2 GAG antibodies in the ALS individuals indicates that full-length Herv-K/HML-2 viral RNA was present in cells of those individuals. Thus, such individuals likely contain cells infected with Herv-K/HML-2 virus. Accordingly, the present invention provides methods for identifying,cells infected with Herv-K/HML-2 virus and methods for monitoring for the presence of Herv-K/HML-2 infected cells.

The invention provides a replication competent Herv-K/HML-2 virus comprising an RNA genome encoding a GAG polypeptide comprising an amino acid sequence of amino acid residues of about 31 to about 93 of the polypeptide herein designated KG-ME-2. The invention also provides various compositions comprising a polynucleotide sequence comprising a nucleotide sequence of nucleotides about 91 to about 279 of the KG-ME-2 nucleotide sequence or a polypeptide comprising an amino acid sequence of amino acid residues about 31 to about 93 of the KG-ME-2 polypeptide. The invention also provides anti-Herv-K/HML-2 antibodies, particularly antibodies which specifically bind to a polypeptide comprising an amino acid sequence of amino acid residues about 1 to about 93 of the KG-ME-2 polypeptide and antibodies which specifically bind to a polypeptide comprising an amino acid sequence of amino acid residues about 31 to about 93 of the KG-ME-2 polypeptide (e.g., SE-HA).

Without wishing to be bound by any particular theory, the results herein presented are consistent with a model of ALS in which infection and/or re-activation of an Herv-K L-2 like agent in spinal column microglia initiates an inflammatory cascade which attracts additional monocytes and/or T cell infiltration leading to further up-regulation of endogenous Herv-K/HML-2 expression and, consequently greater inflammation. Eventually, circulating Herv-K/HML-2 levels are sufficient to initiate a humoral immune response. The humoral immune response may then result in immune complex formation and antibody deposits within the spinal column and such deposits may further drive the inflammatory process. Accordingly, down regulation of Herv-K/HML-2 antigen expression and/or Herv-K/HML-2 infection may be effective in reducing inflammation associated with ALS.

The invention provides methods for decreasing expression of Herv-K/HML-2 in an individual. The invention also provides methods for ameliorating a symptom of ALS by decreasing Herv-K/HML-2 expression, including, for example, ameliorating ongoing inflammation and/or microglial activation associated with ALS. The invention also provides methods for decreasing production of Herv-K/HML-2 virus in an individual with ALS through administration of a retroviral inhibitor specific for Herv-K/HML-2, which alone or in conjunction with other treatment modalities may delay development of and/or ameliorate one or more symptoms of ALS. The invention also provides methods for decreasing production of Herv-K/HML-2 virus in an individual with ALS through administration of a vaccine comprising Herv-K/HML-2 GAG and/or ENV polypeptides, which alone or in conjunction with other treatment modalities may delay development of and/or ameliorate one or more symptoms of ALS.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989, Cold Spring Harbor Laboratory Press, Cold Spring Harbor, N.Y.); Oligonucleotide Synthesis (Gait, ed., 1984); Animal Cell Culture (Freshney, ed., 1987); Handbook of Experimental Immunology (Weir et al., eds.); Gene Transfer Vectors for Mammalian Cells (Miller et al., eds., 1987); Current Protocols in Molecular Biology (Ausubel et al., eds., 1995); PCR: The Polymerase Chain Reaction, (Rullis et al., eds., 1994); Current Protocols in Immunology (Coligan et al., eds., 1991); The Immunoassay Handbook (Wild, ed., Stockton Press NY, 1994); Antibodies: A Laboratory Manual (Harlow et al., 1988, Cold Spring Harbor Laboratory Press, Cold Spring Harbor, N.Y.); Bioconjugate Techniques (Hermanson, ed., Academic Press, 1996); and Methods of Immunological Analysis (Masseyeff et al., eds., Weinheim: VCH Verlags gesellschaft mbH, 1993). In general, the flow cytometric methods used in the examples described herein and appropriate for use in the invention are well known in the art. See, for example, Flow Cytometry: A Practical Approach, 2nd ed. (Ormerod, ed., Oxford University Press, 1997); Handbook of Flow Cytometry Methods (Robinson, ed., John Wiley & Sons, 1993); Current Protocols in Cytometry (Robinson, ed., John Wiley & Sons, October 1997, with periodic updates); Becton Dickinson Cytometry Source Book, Becton Dickinson Immunocytometry Systems, 1998, with periodic updates, San Jose, Calif.).

Definitions

“Amyotrophic lateral sclerosis” or “ALS” are terms understood in the art and as used herein to denote a progressive neurodegenerative disease that affects upper motor neurons (motor neurons in the brain) and/or lower motor neurons (motor neurons in the spinal cord) and results in motor neuron death. As used herein, the term “ALS” includes all of the classifications of ALS known in the art, including, but not limited to classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking), Progressive Muscular Atrophy (PMA, typically affecting only the lower motor neurons) and familial ALS (a genetic version of ALS).

As used interchangeably herein, the terms “Herv-K/HML-2” and “Herv-K” and “HML-2” are meant to refer to human endogenous retroviruses (Hervs) that belong to a specific subgroup of human endogenous mouse mammary tumor virus (MMTV)-like retroviruses (HMLs). Hervs are divided into different families based on degrees of nucleic acid sequence similarity to other retroviruses and other features such as the tRNA primer that is used in replicating the viral genome. For example, Herv-K uses a lysine tRNA in its replication and Herv-W uses a tryptophan tRNA in its replication. See, for example, Urnovitz et al. (1996) Clin. Microbiol. Reviews 9:72-99. Herv-K, which belongs to the group HML-2, was found to be relatively uninterrupted by stop codons in the open reading frames (ORFs) for all genes. See, for example, Ono et al. (1986) J. Virol. 60:589-598 and Medstrand et al. (1993) J. Virol. 67:6778-6787.

By “Herv-K/HML-2 associated disease or disorder” is meant a disease or disorder associated with the expression of Herv-K/HML-2 and/or the infection of cells by Herv-K/HML-2. A Herv-K/HML-2 associated disease or disorder is associated particularly with the expression of Herv-K/HML-2 GAG polypeptide and/or ENV polypeptide including, but not limited to, a polypeptide comprising the amino acid sequence of KG-ME-2, or a portion thereof. ALS is an example of such a Herv-K/HML-2-associated disease. Some Herv-K/HML 2 associated diseases or disorders are caused or perpetuated in whole or in part due to uncontrolled expression of Herv-K/HML-2 proviruses including, for example, certain types of germ cell tumors including seminomas (Sauter et al. (1995) J. Virol. 69:414-421, Boller et al. (1997) J. Virol. 71:4581-4588).

As used interchangeably herein, the terms “nucleic acid” and “polynucleotide” and “oligonucleotide” include single-stranded DNA (ssDNA), double-stranded DNA (dsDNA), single-stranded RNA (ssRNA) and double-stranded RNA (dsRNA), cDNA, DNA-RNA hybrids, modified oligonucleotides and oligonucleosides or combinations thereof The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, deoxythymidine, and deoxycytidine. A nucleotide is a phosphate ester of a nucleoside.

It is understood that reference to DNA in the context of a Herv-K/HML-2 RNA virus particle, and other RNA virus particles, is meant to refer to a DNA sequence as it would be produced from the genomic RNA, without limitation as to the method of making the DNA sequence. Similarly, its is understood that DNA sequences of Herv-K/HML-2 RNA virus sequences, and other RNA viruses, encompasses the corresponding RNA, where uracil (U) is substituted for thymine (I), and further encompasses the complementary strand and its corresponding RNA sequence. DNA in the context of the endogenous retrovirus Herv-K/HML-2 provirus and other endogenous retrovirus proviruses, is meant to refer to the provirus DNA sequence as it is found integrated into the host DNA.

The terms “polypeptide” and “protein”, used interchangeably herein, refer to a polymeric form of amino acids of any length, which can include coded and non-coded amino acids, chemically or biochemically modified (e.g., post-translational modification such as glycosylation) or derivatized amino acids, polymeric polypeptides, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. Polypeptides can also be modified to, for example, facilitate attachment to a support (e.g., to a solid or semi-solid support, to a support for use as an array, and the liked).

The term “peptide” are polypeptides that are of sufficient length and composition to effect a biological response, e.g., antibody production or cytokine activity whether or not the peptide is a hapten. Typically, the peptides are at least six amino acid residues in length. The term “peptide” further includes modified amino acids (whether or not naturally or non-naturally occurring), such modifications including, but not limited to, phosphorylation, glycosylation, pegylation, lipidization and methylation.

As used herein, a polynucleotide “derived from” a designated sequence refers to a polynucleotide sequence which is comprised of a sequence of approximately at least about 6 contiguous nucleotides, at least about 8 nucleotides, at least about 10-12 contiguous nucleotides, and at least about 15-20 contiguous nucleotides corresponding to a region of the designated nucleotide sequence. “Corresponding” means homologous to, identical to or complementary to the designated sequence. Particularly, the sequence of the region from which the polynucleotide is derived is homologous or identical to or complementary to a sequence which is unique to a Herv-K/HML-2 genome. Regions from which typical polynucleotide sequences may be “derived” include, but are not limited to, for example, regions encoding specific epitopes, as well as non-transcribed and/or non-translated regions.

The derived polynucleotide is not necessarily physically derived from the nucleotide sequence shown, but may be generated in any manner, including for example, chemical synthesis or DNA replication or reverse transcription or transcription. In addition, combinations of regions corresponding to that of the designated sequence may be modified in ways known in the art to be formulated with an intended use.

Similarly, a polypeptide or amino acid sequence “derived from” a designated nucleic acid sequence refers to a polypeptide having an amino acid sequence identical to that of a polypeptide encoded in the sequence, or a portion thereof, wherein the portion consists of at least 3-5 contiguous amino acids, and more preferably at least 8-10 contiguous amino acids, and even more preferably at least 11-15 contiguous amino acids, or which is immunologically identifiable with a polypeptide encoded in the sequence. This terminology also includes a polypeptide expressed from a designated nucleic acid sequence.

A recombinant or derived polypeptide is not necessarily translated from a designated nucleic acid sequence, for example, the sequence encoding Herv-K/HML-2 GAG polypeptide as set forth in the nucleotide sequence designated KG-ME-2, or from a Herv-K/HML-2 genome. A recombinant or derived polypeptide, e.g., Herv-K/HML-2 GAG, may be generated in any manner, including for example, chemical synthesis, or expression of a recombinant expression system, or isolation from Herv-K/HML-2 virus, including mutated Herv-K/HML-2 virus. A recombinant or derived polypeptide may include one or more analogs of amino acids or unnatural amino acids in its sequence. Methods of inserting analogs of amino acids into a sequence are known in the art. It also may include one or more labels, which are known to those of skill in the art.

The term “recombinant polynucleotide” as used herein intends a polynucleotide of genomic, cDNA, semisynthetic, or synthetic origin which, by virtue of its origin or manipulation: (1) is not associated with all or a portion of a polynucleotide with which it is associated in nature, (2) is linked to a polynucleotide other than that to which it is linked in nature, or (3) does not occur in nature.

The term “3′” generally refers to a region or position in a polynucleotide or oligonucleotide 3′ (downstream) from another region or position in the same polynucleotide or oligonucleotide. The term “3′ end” refers to the 3′ terminus of the polynucleotide.

The term “5′ ” generally refers to a region or position in a polynucleotide or oligonucleotide 5′ (upstream) from another region or position in the same polynucleotide or oligonucleotide. The term “5′ end” refers to the 5′ terminus of the polynucleotide.

“Operably linked” refers to ajuxtaposition wherein the components so described are in a relationship permitting them to function in their intended manner. A control sequence “operably linked” to a coding sequence is ligated in such a way that expression of the coding sequence is achieved under conditions compatible with the control sequences.

An “open reading frame” (ORF) is a region of a polynucleotide sequence which encodes a polypeptide. This region may represent a portion of a coding sequence or a total coding sequence.

A “coding sequence” is a polynucleotide sequence which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, and recombinant polynucleotide sequences.

An “antibody titer”, or “amount of antibody”, which is “elicited” by an antigen refers to the amount of a given antibody measured at a time point in a particular amount or volume of a sample.

By “specifically binds” as used in the context of a Herv-K/HML-2 polynucleotide (e.g., nucleic acid probe) or polypeptide (e.g., as detected using an antibody that specifically binds the polypeptide) means that the Herv-K/HML-2 polynucleotide or polypeptide can be used as a marker for Herv-K/HML-2 expression so that Herv-K/HML-2 expression is detected so as to be distinguished from non-Herv-K/HML-2 polynucleotides or non-Herv-K/HML-2 polypeptides. For example, a specific Herv-K/HML-2 polynucleotide is one that can be used to specifically detect Herv-K/HML-2 nucleic acid (in, e.g., nucleic acid amplification- or hybridization-based assays) so as to differentiate Herv-K/HML-2 nucleic acid from non-Herv-K/HML-2 nucleic acid. Similarly, a specific Herv-K/HML-2 polypeptide is a polypeptide that can be detected (e.g., by antibody-based assay) so as to specifically detect Herv-K/HML-2 polypeptide in a sample and differentiate Herv-K/HML-2 polypeptide from non-Herv-K/HML-2 polypeptides. Similarly, an Herv-K/HML-2-specific antibody is an antibody that can be used in detection of a Herv-K/HML-2-specific polypeptide or Herv-K/HML-2-specific epitope so as to specifically detect Herv-K/HML-2 in a sample and differentiate Herv-K/HML-2 polypeptide from non-Herv-K/HML-2 polypeptides.

As used herein, the term “probe” refers to a molecule useful in specific detection of Herv-K/HML-2 expression. “Probes” thus include, a polynucleotide that specifically hybridizes to a Herv-K/HML-2 polynucleotide in a target region, due to complementarity of at least one sequence in the probe with a sequence in the target region. Unless specifically noted otherwise, probes encompass primers (e.g., primers used in PCR-based amplification of a region adjacent to a target region). “Probes” also include antibodies that specifically bind a Herv-K/HML-2 polypeptide, as well as Herv-K/HML-2 polypeptides that specifically bind anti-Herv-K/HML-2 antibodies. The meaning of probe will be readily apparent to the ordinarily skilled artisan from the context of the use of the term.

An “Herv-K/HML-2-specific probe” is a molecule (e.g., nucleic acid, antibody, polypeptide) that specifically binds a Herv-K/HML-2-specific probe target. Exemplary Herv-K/HML-2-specific probes include nucleic acid that specifically hybridizes to a sequence of Herv-K/HML-2, nucleic acid primer pairs that facilitate amplification of a Herv-K/HML-2-specific nucleic acid sequence, an anti-Herv-K/HML-2 GAG antibody that specifically binds the GAG of Herv-K/HML-2, a Herv-K/HML-2 GAG polypeptide that specifically binds an anti-Herv-K/HML-2 GAG antibody, an anti-Herv-K/HML-2 ENV antibody that specifically binds the ENV of Herv-K/HML-2, and a Herv-K/HML-2 ENV polypeptide that specifically binds an anti-Herv-K/HML-2 ENV antibody.

As used herein, the term “target region” as used in the context of a nucleic acid probe refers to a region of the nucleic acid which is to be amplified and/or detected. “Target region” as used in the context of antibody-polypeptide (antibody-antigen) complex formation refers to a region of the polypeptide that forms the epitope specifically bound by the antibody.

“Probe target” as used herein is meant to refer to a molecule to which a Herv-K/HML-2-specific probe specifically binds. As used herein, a Herv-K/HML-2 probe target is a molecule that can be used to indicate Herv-K/HML-2 expression. The probe target can be nucleic acid (RNA or DNA), an antibody or a polypeptide. Combinations of probes and probe targets described herein will be readily apparent to one of ordinary skill in the art upon reading the present specification.

As used herein, the term “viral nucleic acid”, which includes Herv-K/HML-2 nucleic acid, refers to nucleic acid from the viral genome, fragments thereof, transcripts thereof, and mutant sequences derived therefrom. Viral nucleic acid can be derived from any source, e.g., synthetic production techniques, recombinant expression techniques, and the like.

As used herein, microglia are cells of macrophage/monocyte origin found in all neural tissues that provide support functions to the actual neurons.

As used herein, the terms “macrophage” and “monocyte” are used interchangeably, as it is understood that in the art the term “monocyte” is often used to describe a circulating mononuclear cell that expresses the CD14 cell surface marker, and when in a tissue this cell is also classified as a macrophage.

As used herein, detecting the “expression of Herv-K/HML-2” generally means detecting a direct product of transcription of Herv-K/HML-2 DNA, e.g., Herv-K/HML-2 RNA, or a downstream product that results from transcription of Herv-K/HML-2 DNA, e.g., a polypeptide encoded by a Herv-K/HML-2 gene, a Herv-K/HML-2 virus particle or an anti-Herv-K/HML-2 antibody that binds a polypeptide encoded by a Herv-K/HML-2 gene. It is understood that an absolute or even relative level of Herv-K/HML-2 expression need not be determined; an observation of expression of Herv-K/HML-2 is sufficient.

An “individual” is a vertebrate, preferably a mammal, more preferably a human. Mammals include, but are not limited to, farm animals, sport animals, rodents, primates, and pets. An “ALS individual” or an “ALS patient” is an individual who is diagnosed as having ALS or is suspected of having ALS by demonstrating ALS-associated symptoms. A “non-ALS individual” is an individual who is not diagnosed as having ALS or not suspected of having ALS. ALS and methods of diagnosing ALS are known in the art and are discussed herein.

As used herein, “biological sample” encompasses a variety of sample types obtained from an individual and can be used in a diagnostic or monitoring assay. The definition encompasses blood, cerebral spinal fluid (CSF), urine and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples. Generally, the particular biological sample will depend on the type of probe target to which the assay is directed. For example, when the probe target is anti-Herv-K/HML-2 antibodies, the biological sample will generally be, or be derived from, a blood sample. In another example, when the probe target is Herv-K/HML-2 RNA, the biological sample may be CSF, or be derived from CSF, or may be a biopsy specimen from an area of neuroinflammation.

A “blood sample” is a biological sample which is derived from blood, preferably peripheral (or circulating) blood. A blood sample may be, for example, whole blood, plasma or serum.

As used herein, methods for “aiding diagnosis” means that these methods assist in making a clinical determination regarding the classification, or nature, of the ALS, and may or may not be conclusive with respect to the definitive diagnosis. Accordingly, a method of aiding diagnosis of ALS can comprise the step of testing for expression of Herv-K/HML-2 in a biological sample from the individual. As described herein, expression of Herv-K/HML-2 genes, particularly expression of the Herv-K/HML-2 gag gene, is associated with sporadic ALS. In various embodiments, expression of Herv-K/HML-2 can be detected by determining the presence of anti-Herv-K/HML-2 antibodies in a biological sample from an individual, preferably a blood sample.

“Development” or “progression” of ALS herein means initial manifestations and/or ensuing progression of the disorder. Development of ALS can be detectable and assessed using standard clinical techniques, such as nerve and muscle biopsy and CNS scanning technologies such as MRI. However, development also refers to disease progression that may be undetectable. For purposes of this invention, development or progression refers to the biological course of the disease state. “Development” includes occurrence, recurrence, and onset. As used herein “onset” or “occurrence” of ALS includes initial onset and/or recurrence.

As used herein, “delaying development” of ALS means to defer, hinder, slow, retard, stabilize, and/or postpone development of the disease. This delay can be of varying lengths of time, depending on the history of the disorder and/or the medical profile of the individual being treated. As is evident to one skilled in the art, a sufficient or significant delay can, in effect, encompass prevention, in that the individual does not develop detectable disease. A method that “delays” development of disease is a method that reduces the extent of the disease in a given time frame, when compared to not using the method. Such comparisons are typically based on clinical studies, using a statistically significant number of subjects, although this knowledge can be based upon anecdotal evidence. “Delaying development” can mean that the extent and/or undesirable clinical manifestations are lessened and/or time course of the progression is slowed or lengthened, as compared to not administering the agent. Thus the term also includes, but is not limited to, alleviation of symptoms, diminishment of extent of disease, stabilized (i.e., not worsening) state of disease, delay or slowing of disease progression, and remission (whether partial or total) whether detectable or undetectable.

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Palliating” a disease or disorder means that the extent and/or undesirable clinical manifestations of a disorder or a disease state are lessened and/or time course of the progression is slowed or lengthened, as compared to not treating the disorder. “Treatment” can also mean prolonging survival as compared to expected survival if not receiving treatment.

As used herein, an “effective amount” or a “sufficient amount” (e.g., of an agent) is an amount (of the agent) that produces a desired and/or beneficial result, including clinical results, and, as such, an “effective amount” or a “sufficient amount” depends upon the context in which it is being applied. An effective amount can be administered in one or more administrations. In some embodiments, an effective amount is an amount sufficient to decrease expression of Herv-K/HML-2 in an ALS patient. An “amount sufficient to decrease expression of Herv-K/HML-2” preferably is able to decrease expression of Herv-K/HML-2 by at least about 25%, preferably at least about 50%, more preferably at least about 75%, and even more preferably at least about 90%. Such decreases may have desirable concomitant effects, such as to palliate, ameliorate, stabilize, reverse, slow or delay progression of disease, delay and/or even prevent onset of disease.

As used herein, the term “agent” means a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic compounds based on various core structures, and these are also included in the term “agent”. In addition, various natural sources can provide compounds, such as plant or animal extracts, and the like. Agents include, but are not limited to, polyamine analogs. Agents can be administered alone or in various combinations.

As used herein, “a”, “an”, and “the” can mean singular or plural (i.e., can mean one or more) unless indicated otherwise.

As used herein, the term “comprising” and its cognates are used in their inclusive sense; that is, equivalent to the term “including” and its corresponding cognates.

### METHODS OF THE INVENTION

The present invention provides methods of aiding diagnosis of ALS, particularly sporadic ALS, comprising determining the presence or absence of Herv-K/HML-2 expression in an individual. The present invention also provides methods of monitoring therapy of ALS in an individual comprising determining expression of Herv-K/HML-2 in a biological sample from the individual. The present invention also provides methods of monitoring development or progression of ALS in a patient with ALS comprising determining Herv-K/HML-2 expression in a biological sample from the ALS patient.

As described herein, expression of Herv-K/HML-2 correlates with an individual having sporadic ALS. In one study, Herv-K/HML-2 expression (as determined by the presence of anti-KG-ME-2 antibodies) correlates with the length of time the individual has been symptomatic for ALS and correlates with low ALS functional rating scores. Accordingly, monitoring for expression of Herv-K/HML-2 may in turn indicate initial responsiveness and/or efficacy, as well as the appropriate dosage of the therapy. It is understood that monitoring therapy means that biological sample(s) are obtained at different times, for example, during application of therapy, and are compared, either with each other, a control, and/or a desired value. In one embodiment, monitoring therapy includes the step of determining the presence, absence or level of Herv-K/HML-2 expression in a biological sample from the individual. In another embodiment, expression of Herv-K/HML-2 in a biological sample determined during and/or at completion of the therapy is generally compared with expression of Herv-K/HML-2 in a control sample and/or with a desired value.

For the purpose of monitoring an ALS therapy in one embodiment, the expression of Herv-K/HML-2 in a sample taken at a particular time from a patient undergoing the therapy and/or a sample taken after or at completion of the therapy is generally compared with expression of Herv-K/HML-2 in a sample taken from the patient prior to the therapy and/or with expression of Herv-K/HML-2 in a sample taken from the patient at a different time point in the therapy. For example, a decrease in expression of Herv-K/HML-2 in the sample taken during therapy as compared to the sample taken prior to or at an earlier time point in therapy would generally be consistent with a positive effect of the ALS therapy.

In one embodiment, for the purpose of monitoring an ALS therapy, expression of Herv-K/HML-2 is assessed by the determining the absence, presence, and/or level of Herv-K/HML-2 expression in a biological sample, such as a blood or CSF sample. For example, the effect of a therapy is determined by comparing the level of Herv-K/HML-2 expression in a biological sample before and during treatment, with a downward trend in Herv-K/HML-2 expression generally being consistent with a positive effect.

In those individuals with ALS, assessment of Herv-K/HML-2 expression in a biological sample, e.g., blood, CSF or a biopsy, may also assist in monitoring development or progression of the disease. Thus, the invention also includes methods of monitoring disease development or progression in an individual with ALS, comprising assaying for Herv-K/HML-2 expression in a biological sample from that individual. Preferably, the individual is “afflicted with” (e.g., diagnosed as having, suffering from and/or displaying one or more clinical symptoms of) ALS.

As expression of Herv-K/HML-2 correlates with an individual having sporadic ALS, monitoring Herv-K/HML-2 expression may provide an indication of changes in the development or progression of the disease. It is understood that monitoring an individual with ALS generally means that biological sample(s) are obtained at different times, for example, over weeks, months and/or years, and are compared with each other, a control, and/or a desired value. In some embodiments of monitoring of ALS, expression of Herv-K/HML-2 is generally consistent with an increase in disease severity and/or rate of progression.

In those individuals considered at high or significant risk of developing ALS, determining expression of Herv-K/HML-2 in a biological sample may also assist in alerting the individual and/or the clinician of possible precursor disease. Thus, the invention also includes methods of monitoring an individual at risk or high risk of developing ALS, comprising assessing for Herv-K/HML-2 expression in a biological sample from that individual. Preferably, the individual is displaying one or more clinical symptoms associated with ALS, or at “risk” for (e.g., having a genetic predisposition for, or family history of, or being environmentally exposed to factors which increase the probability of acquiring) ALS. In monitoring an individual at risk or high risk of developing ALS, expression of Herv-K/HML-2 in a biological sample is generally consistent with an increase in risk of development of a symptom of ALS disease.

It is understood that monitoring an individual at (high) risk generally, but not necessarily, means that biological sample(s) are obtained at different times, for example, over weeks, months and/or years, and are compared with each other, a control, and/or a desired value. In one embodiment, monitoring an individual at (high) risk includes the step of assessing for expression of Herv-K/HML-2 in a biological sample, e.g. a blood sample or a CSF sample.

For the purpose of monitoring a therapy, monitoring disease development or progression, or monitoring an individual at (high) risk, generally expression of Herv-K/HML-2 in a sample may be compared with expresssion of Herv-K/HML-2 in samples taken from healthy individuals or from non-ALS patients, matched where necessary for sex and/or age. Alternatively, results of these indicia can be compared with expression of Herv-K/HML-2 from samples taken from the same monitored individual at various time points. A difference or change in Herv-K/HML-2 expression or in the level of Herv-K/HML-2 expression from the ALS samples when compared to that of the non-ALS samples generally correlates with a change in the disease development or activity. For example, the presence and/or an increase in Herv-K/HML-2 expression correlates with an increase in ALS progression.

In combination with one or more other disease indicators, the detection of Herv-K/HML-2 expression in an individual may aid in diagnosis or prediction of ALS. The differential diagnosis will include any condition associated with ALS as a causative or consequential effect, with the ultimate diagnosis being the responsibility of the managing physician or clinician. Accordingly, the invention includes methods of aiding diagnosis of ALS. These methods generally comprise the step of assessing for Herv-K/HML-2 expression in a biological sample from the individual suspected of having ALS.

Circulating monocytes isolated from 2 of 3 patients with ALS appear to express polypeptides comprising the KG-ME-2 amino acid sequence since these cells were significantly stained with labeled IgG purified from ALS sera that contained antibodies reactive to the KG-ME-2 fragment of Herv-K/HML-2. Since retroviral GAG proteins require full-length retroviral RNA in order to be produced, the existence of anti-Herv-K/HML-2 GAG antibodies in the ALS individuals indicates that full-length Herv-K/HML-2 viral RNA was present in cells of those individuals. Thus, such individuals likely contain cells, including monocytes, infected with Herv-K/HML-2 virus.

The invention provides methods for decreasing expression of Herv-K/HML-2 and/or suppressing Herv-K/HML-2 viral replication in a individual in need thereof, for example, in an individual with a Herv-K/HML-2 associated disease or disorder. The invention also provides methods for ameliorating a symptom of ALS through decreasing expression of Herv-K/HML-2 expression in the individual. In some embodiments, expression of Herv-K/HML-2 is sufficiently decreased in the individual such that ongoing inflammation and/or microglial activation associated with ALS is decreased and at least one symptom of ALS is ameliorated.

The invention provides methods for decreasing production of Herv-K/HML-2 virus in an individual with ALS through administration of a vaccine comprising a Herv-K/HML-2 polypeptide, e.g., a Herv-K/HML-2 GAG and/or ENV polypeptide, to the individual, which alone or in conjunction with other treatment modalities may delay development of and/or ameliorate one or more symptoms of ALS. Administration of such a polypeptide as a vaccine may result, for example, in decreasing viral titer in the individual, in reducing expression of Herv-K/HML-2 in the individual, in a destruction of cell producing virus particles or expressing the polypeptide, and in ameliorating one or more symptoms of ALS.

The invention also provides methods for decreasing production of Herv-K/HML-2 virus in an individual with ALS through administration of a retroviral inhibitor specific for Herv-K/HML-2, which alone or in conjunction with other treatment modalities may delay development of and/or ameliorate one or more symptoms of ALS.

Symptoms associated with ALS are known in the art (see, for example, Rowland et al. (2001) N. Engl. J Med. 344:1688-1700). Such symptoms include, but are not limited to, muscle weakness, decrease in muscle strength and coordination, paralysis, muscle cramps, voice changes and/or hoarseness, speech impairment, difficulty swallowing, gagging or choking easily, difficulty breathing, muscle contractions, muscle atrophy, urinary frequency/urgency, and ankle, feet and leg swelling. ALS symptoms indicated upon neuromuscular examination may include, for example, weakness beginning in one limb or in proximal groups (e.g., shoulders, hips), muscle tremors, spasms, fasciculation, muscle atrophy, clumsy gait and abnormal reflexes. With respect to disease progression, multiple rating scales (i.e., indices of clinical function) have been established and are known in the art for ALS.

The agents that decrease Herv-K/HML-2 expression and/or suppress Herv-K/HML-2 viral replication, including but not limited to those agents identified as described herein, can be used in these methods to treat individuals with a Herv-K/HML-2 associated disease or disorder. Since, Herv-K/HML-2 carries a reverse transcriptase and protease enzyme, both of which have been successful targets for anti-HIV therapeutics, agents that act in a similar manner but effective against Herv-K/HML-2 may find particular use in treatment of Herv-K/HML-2 infection and/or amelioration of a symptom of a Herv-K/HML-2 associated disease or disorder, such as ALS.

The invention also is directed to methods for identifying agents that decrease Herv-K/HML-2 expression and/or suppress Herv-K/HML-2 viral replication. In some embodiments, agents identified that decrease Herv-K/HML-2 expression and/or suppress Herv-K/HML-2 viral replication are further tested for their specificity toward Herv-K/HML-2. In some embodiments, the invention is directed to methods for identifying agents that decrease Herv-K/HML-2 GAG expression, in particular, methods for identifying agents that decrease expression of a polypeptide comprising the Herv-K/HML-2 GAG fragment designated KG-ME-2. In some embodiments, the invention is directed to methods for identifying agents that decrease Herv-K/HML-2 ENV expression. Accordingly, the invention provides methods of screening for agents effective for ameliorating a symptom of ALS.

In these methods, the term “agent” refers to any molecule, e.g., protein or pharmaceutical, which is amenable for screening for anti-Herv-K/HML-2 activity (e.g., gene or polypeptide expression, activity in inhibiting replication, infection, or other aspect of Herv-K/HML-2 infection and propagation). Generally, pluralities of assay mixtures are run in parallel with different agent concentrations to detect differential responses to the various concentrations. Typically, one of these concentrations serves as a negative control, i.e., at zero concentration or below the level of detection.

Candidate agents encompass numerous chemical classes, though typically they are organic molecules, and are generally small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to peptides, saccharides, fatty acids, steroids, pheromones, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

Various screening methods useful in the present invention are known by those of skill in the art. Generally, the agents for decreasing Herv-K/HML-2 expression are tested at a variety of concentrations, for their, effect on reducing expression of Herv-K/HML-2 (e.g., RNA and/or polypeptides) in cell culture systems which support Herv-K/HML-2 expression, and then for reducing expression of Herv-K/HML-2 (and a low level of toxicity) in an animal model system. The anti-Herv-K/HML-2 expression agents which may be tested for efficacy by these methods are known in the art, and include, for example, those which interact with Herv-K/HML-2 transcription, translation, and/or cellular components which are necessary for the processing of Herv-K/HML-2 RNA and/or polypeptide to generate a Herv-K/HML-2 antigen. Typical anti-gene expression agents may include, for example, inhibitors of translation that are specific for a particular RNA, such as those that include antisense polynucleotide technology.

Antisense polynucleotides molecules, which are comprised of a complementary nucleotide sequence which allows them to hybridize specifically to designated regions of Herv-K/HML-2 genomes or RNAs, is an example of an anti-Herv-K/HML-2 expression agent of interest that can be identified through screening assays according to the invention. Antisense polynucleotides may include, for example, molecules that will block protein translation by binding to mRNA, or may be molecules which prevent replication of viral RNA by transcriptase. They may also include molecules which carry agents (non-covalently attached or covalently bound) which cause the Herv-K/HML-2 RNA to be inactive by causing, for example, scissions in the viral RNA, such as ribozymes and the like.

Antisense molecules which are to hybridize to Herv-K/HML-2 derived RNAs may be designed based upon the sequence information of the Herv-K/HML-2 polynucleotide sequences known in the art and provided herein. The anti-Herv-K/HML-2 expression agents and/or anti-Herv-K/HML-2 viral agents based upon anti-sense polynucleotides for Herv-K/HML-2 may be designed to bind with high specificity, to be of increased solubility, to be stable, and to have low toxicity. Hence, they may be delivered in specialized systems, for example, liposomes, or by gene therapy. In addition, they may include analogs, attached proteins, substituted or altered bonding between bases, and the like. Other types of drugs having anti-Herv-K/HML-2 expression and/or anti-Herv-K/HML-2 viral activity may be based upon polynucleotides which “mimic” important control regions of the Herv-K/HML-2 genome, and which may be therapeutic due to their interactions with key components of the system responsible for viral expression, viral infectivity and/or replication.

Generally, the anti-viral agents are tested at a variety of concentrations, for their effect on preventing viral replication in cell culture systems which support viral replication, and then for an inhibition of infectivity or of viral pathogenicity (and a low level of toxicity) in an animal model system. Exemplary methods include, but are not necessarily limited to, assays to determine the effect of the agent upon viral ID₅₀ or upon the ability of the virus to induce cell plaque formation. The methods and compositions provided herein for detecting Herv-K/HML-2 polypeptides and polynucleotides are useful for screening of anti-viral agents in that they provide an alternative, and sensitive, means for detecting the agent's effect on viral replication other than the cell plaque assay or ID₅₀ assay.

For example, the Herv-K/HML-2 polynucleotide probes described herein may be used to quantitate the amount of viral nucleic acid produced in a cell culture. This could be accomplished, for example, by hybridization or competition hybridization of the infected cell nucleic acids with a labeled Herv-K/HML-2-polynucleotide probe. For example, also, anti-Herv-K/HML-2 antibodies may be used to identify and quantitate Herv-K/HML-2 antigen(s) in the cell culture utilizing the immunoassays described herein. In addition, since it may be desirable to quantitate Herv-K/HML-2 antigens in the infected cell culture by a competition assay, the Herv-K/HML-2 polypeptides described herein are useful in these competition assays. Generally, a recombinant Herv-K/HML-2 polypeptide would be labeled, and the inhibition of binding of this labeled polypeptide to an Herv-K/HML-2 polypeptide due to the antigen produced in the cell culture system would be monitored. Moreover, these techniques are particularly useful in cases where the Herv-K/HML-2 may be able to replicate in a cell line without causing cell death.

The anti-viral agents which may be tested for efficacy by these methods are known in the art, and include, for example, those which interact with virion components and/or cellular components which are necessary for the binding and/or replication of the virus. Typical anti-viral agents may include, for example, inhibitors of virion polymerase and/or protease(s) necessary for cleavage of the precursor polypeptides. Other anti-viral agents may include those which act with nucleic acids to prevent viral replication, for example, anti-sense polynucleotide, etc.

Exemplary Herv-K/HML-2 anti-viral agents include those that inactivate the virus (e.g., by treatment of an instrument or biological material (e.g., blood, tissue) prior to use), inhibit Herv-K/HML-2 entry into a host cell, inhibit Herv-K/HML-2 replication, or otherwise disrupt or interfere with Herv-K/HML-2-associated pathogenesis. Those agents that allow growth and proliferation of the infected cell while inhibiting viral replication are of particular interest, with agents that facilitate inhibition of growth of infected cells, up to and including death of such cells, also being of interest.

Since antibody-antigen deposits can be detrimental to various organs and tissues, including neural tissue, the invention also provides methods for reducing and/or suppressing the level of anti-Herv-K/HML-2 antibodies in an individual in need thereof, for example, an individual with ALS. Methods for reducing levels of antibodies, including disease or disorder-associated antibodies are known in the art. In some embodiments, the invention provides methods for inducing specific B cell anergy to a particular immunogen (e.g., Herv-K/HML-2 GAG polypeptide) using methods described, for example, in U.S. Pat. No. 6,060,056. In such methods, an analog of the immunogen that (a) binds specifically to an antibody to which the immunogen binds specifically (e.g., an anti-Herv-K/HML-2 GAG antibody) and (b) lacks T cell epitopes is conjugated to a nonimmunogenic valency platform and administered to the individual. Administration of such a composition results in B cell anergy to the particular immunogen and, thus, improvement or elimination of the antibody-mediated condition being address.

In some embodiments, the invention provides methods for reducing the concentration of anti-Herv-K/HML-2 antibodies in the blood of an individual through the use of Herv-K/HML-2 polypeptides, in particular Herv-K/HML-2 GAG polypeptides, as an immunoadsorbant for the anti-Herv-K/HML-2 antibodies. For example, PCT Pub. No. WO 00/33887 describes methods for reducing levels of circulating antibodies in an individual through administration of an effective amount of an epitope-presenting moiety, such as an epitope conjugated to a valency platform molecule.

Methods for reducing the level of anti-Herv-K/HML-2 antibodies in the blood of an individual includes the use of apheresis or plasmapheresis techniques which involve affinity adsorption of the anti-viral antibodies from isolated plasma and then the reintroduction of the treated plasma to the individual. Accordingly, the invention provides methods for reducing the concentration of anti-Herv-K/HML-2 antibodies in the blood of an individual through the use of Herv-K/HML-2 polypeptides, in particular Herv-K/HML-2 GAG polypeptides, as an immunoadsorbant for the anti-Herv-K/HML-2 antibodies.

PCT Pub. No. WO 00/33887, for example, describes an ex vivo method for reducing antibodies in which an individual's blood, or an antibody-containing component thereof, is treated extracoporeally with an epitope presenting carrier. Antibody-epitope presenting carrier complexes, if any, are removed and the treated blood is returned to the individual.

Affinity adsorption apheresis is known in the art and described generally, for example, in Nilsson et al. (1981) Blood 58:38-44; Christie et al. (1993) Transfusion 33:234-242; Suzuki et al. (1994) Autoimmunity 19:105-112; U.S. Pat. No. 5,733,254; Richter et al. (1993) Metabol. Clin. Exp. 42:888-894. For example, U.S. Pat. No. 6,464,976 describes reduction in the concentration of antiviral antibodies in plasma through plasmapheresis and immunoaffinity of the antiviral antibodies to adsorb the antibodies out of the plasma.

The term “plasmapheresis” refers to an apheresis procedure in whereby blood removed from a mammal is separated into plasma and cellular blood components, the plasma being isolated for further processing. The principles and practice of apheresis are well known in the art. Standard procedures for apheresis are described in Apheresis: Principles and Practice commercially available from the American Association of Blood Banks (Bethesda, Md.). Plasmapheresis is generally performed in the clinical arena using continuous flow centrifugal separators, which separate cells by density; flat-sheet and intralumenal hollow fiber membrane devices, which operate by tangential flow microfiltration; and rotating membrane devices, which enhance microfiltration flux by inducing Taylor vortices. Such devices are commercially available and are well known in the literature, see, e.g. Plasmapheresis: Therapeutic Applications and New Techniques, Nose Y, et al., Raven Press, New York (1983); U.S. Pat. No. 5,783,085; U.S. Pat. No. 5,846,427; U.S. Pat. No. 5,919,369.

Assays for Detection of Herv-K/HML-2 Expression in a Sample

methods of the invention are based on determining the absence, presence and/or level of Herv-K/HML-2 expression in a biological sample of an individual. To determine expression of Herv-K/HML-2, a biological sample is assayed for the presence of a direct and/or downstream product of transcription of Herv-K/HML-2 DNA. Accordingly, in some embodiments, methods of the invention involve assaying biological samples suspected of containing evidence of Herv-K/HML-2 expression. Such methods generally involve assays that are based upon detection of a Herv-K/HML-2 probe target, such as Herv-K/HML-2 RNA, detection of Herv-K/HML-2 polypeptides, or detection of anti-Herv-K/HML-2 antibodies.

Accordingly, in some embodiments, expression of Herv-K/HML-2 can be determined using a Herv-K/HML-2-specific probe to detect Herv-K/HML-2 RNA in a sample, e.g. CSF or a biopsy sample. In other embodiments, expression of Herv-K/HML-2 can be determined using a Herv-K/HML-2-specific probe to detect Herv-K/HML-2 polypeptides in a sample, e.g. CSF or a biopsy sample. Expression of Herv-K/HML-2 can also be determined using a Herv-K/HML-2-specific probe to detect anti-Herv-K/HML-2 antibodies in a sample. The presence of anti-Herv-K/HML-2 antibodies indicates that the immune system of the individual has at some time been exposed to a Herv-K/HML-2 antigen, e.g., a Herv-K/HML-2 polypeptide, Herv-K/HML-2 virus or Herv-K/HML-2 RNA. Accordingly, presence of anti-Herv-K/HML-2 antibodies is an indication of, and marker for, expression of Herv-K/HML-2 DNA.

It will be readily apparent upon reading of the present specification that the assays described herein can be conducted as, or modified to be conducted as, in vitro or in vivo assays, and may be either cell-free (e.g., in vitro binding assays using polynucleotides isolated from or produced from nucleic acid of a biological sample) or cell-based (e.g., screening of whole cells suspected of expressing Herv-K/HML-2). In general, all assays are conducted under conditions, and for a period of time, sufficient to allow for specific binding of an Herv-K/HML-2-specific probe (e.g., nucleic acid probe, antibody probe, polypeptide probe) to an Herv-K/HML-2 probe target, e.g., to provide for detection of Herv-K/HML-2 probe target at a detectable level above background. The assays can include various positive and/or negative controls, the nature of which will be readily apparent to the ordinarily skilled artisan upon reading the present specification. Various aspects of the assays for detection are described herein in more detail.

Biological Samples for Detection Assays

A biological sample of use in the invention is any suitable sample suspected of containing an indication or evidence of Herv-K/HML-2 expression, such as, for example, a Herv-K/HML-2 viral particle, Herv-K/HML-2 RNA, Herv-K/HML-2 polypeptide, anti-Herv-K/HML-2 antibody, a cell expressing Herv-K/HML-2 RNA, polypeptide or anti-Herv-K/HML-2 antibody, and the like. Exemplary samples of interest for assaying include, but are not necessarily limited to, biological samples such as cerebral spinal fluid (CSF), blood, blood derivatives, serum, plasma, urine, platelets, mammalian cells (particularly mammalian lymphocytes, more particularly mammalian macrophages, monocytes, and/or microglia, with human cells being of particular interest), tissues (e.g., biopsy or prior to transplant or other transfer to another subject), and the like.

As demonstrated in the examples presented herein, circulating monocytes (CD14+) from individuals with ALS were found to express Herv-K/HML-2. Accordingly, the fraction of CD 14+/Herv-K/HML-2 expressing monocytes could be monitored as an indication of the extent of disease, with greater Herv-K/HML-2 expression in the monocytes and/or greater numbers of Herv-K/HML-2 expressing monocytes generally indicating more severe disease. The fraction of CD14+/Herv-K/HML-2 expressing monocytes could also be monitored in the methods for monitoring ALS therapy with decreased Herv-K/HML-2 expression and/or decreased numbers of Herv-K/HML-2 expressing monocytes generally indicating treatment efficacy.

As will be readily appreciated by the ordinarily skilled artisan, the specific assay selected will vary according to the source of sample and the entity to be detected (e.g., viral particle, nucleic acid, polypeptide, antibody). Examples of various types of assays are provided herein. Of particular interest are assays that can be readily conducted in a clinic or in the field, without the need for special tools or detection instruments.

The biological samples to be analyzed are maintained in appropriate conditions prior to analysis so that the Herv-K/HML-2 expression product or target, if present in the sample, are detectable at time of analysis.

Detection of Herv-K/HML-2 expression in a subject can also indicate that the subject has, or is at risk of developing, a Herv-K/HML-2-associated disease, such as ALS or a germ cell tumor, such as a seminoma.

Detection of Herv-K/HML-2 expression in a biological sample indicates that the individual from which the sample was obtained may be producing Herv-K/HML-2 viral particles and contain an infectious Herv-K/HML-2 genome. Accordingly, biological material from which the biological sample was obtained should not be used for the purpose of transfer to another subject, as such transfer may result in spread of infectious Herv-K/HML-2 viral particles to the recipient.

Exemplary methods for detection of Herv-K/HML-2 expression according to the invention are described herein.

Methods of Detecting Herv-K/HML-2 Nucleic Acid

Any suitable qualitative or quantitative methods known in the art for detecting specific Herv-K/HML-2 RNA can be used to detect Herv-K/HML-2 expression. For example, Herv-K/HML-2 RNA in cells can be measured by various techniques known in the art including, but not limited to, S1 nuclease analysis, ribonuclease protection assay, primer extension assay, RNA blot analysis (e.g., northern and/or slot blot hybridization) and reverse transcriptase-PCR (RT-PCR), as described, for example, in Ausubel et al., eds., 1995, supra. In addition, Herv-K/HML-2 RNA can be detected by in situ hybridization in tissue sections, using methods that detect single base pair differences between hybridizing nucleic acid (e.g., using the Invader technology described in, for example, U.S. Pat. No. 5,846,717) and other methods well known in the art. For detection of Herv-K/HML-2 RNA in blood or blood-derived samples, RT-PCR based methods are preferred.

Using Herv-K/HML-2 RNA as a basis, with Herv-K/HML-2 GAG and/or ENV polypeptide-encoding RNA being of particular interest, nucleic acid probes (e.g., including oligomers of at least about 8 nucleotides or more) can be prepared, either by excision from recombinant polynucleotides or synthetically, which probes hybridize with the Herv-K/HML-2 nucleic acid, and thus are useful in detection of Herv-K/HML-2 expression in a sample, and identification of individuals which express Herv-K/HML-2, as well as monitoring expression of Herv-K/HML-2 in individuals. The probes for Herv-K/HML-2 polynucleotides (natural or derived) are of a length or have a sequence which allows the detection of unique viral sequences by hybridization. While about 6-8 nucleotides may be useful, longer sequences may be preferred, eg., sequences of about 10-12 nucleotides, or about 20 nucleotides or more. Nucleic acid probes can be prepared using routine methods, including automated oligonucleotide synthetic methods.

Preferably, in some embodiments, these sequences will derive from the 5′ end of the GAG-encoding gene and/or regions which lack heterogeneity among Herv-K/HML-2 viral isolates. In some embodiments, these sequences will derive from the ENV-encoding gene and/or regions which lack heterogeneity among Herv-K/HML-2 viral isolates. In some instances, a complement to any portion of the Herv-K/HML-2 genome specific for Herv-K/HML-2 RNA will be satisfactory, e.g., a portion of the Herv-K/HML-2 genome that allows for distinguishing Herv-K/HML-2 RNA from other viral RNAs that may be present in the sample (e.g., to distinguish the Herv-K/HML-2 RNA from RNA of another endogenous retrovirus). For use as probes, complete complementarity is desirable, though it may be unnecessary as the length of the fragment is increased.

For use of such probes as diagnostics, the biological sample to be analyzed, such as a tissue biopsy, CSF, blood or serum, may be treated, if desired, to extract the RNA contained therein. The resulting RNA from the sample may be subjected to gel electrophoresis or other size separation techniques; alternatively, the RNA sample may be dot blotted without size separation. The probes are usually labeled with a detectable label. Suitable labels, and methods for labeling probes are known in the art, and include, for example, radioactive labels incorporated by nick translation or kinasing, biotin, fluorescent probes, and chemiluminescent probes. The RNA extracted from the sample is then treated with the labeled probe under hybridization conditions of suitable stringencies.

The probes can be made completely complementary to the Herv-K/HML-2 genome or portion thereof (e.g., to all or a portion of a sequence encoding a Herv-K/HML-2 GAG and/or ENV polypeptide). Therefore, usually high stringency conditions are desirable in order to prevent or at least minimize false positives. However, conditions of high stringency should only be used if the probes are complementary to regions of the viral genome which lack heterogeneity among Herv-K/HML-2 viral isolates. The stringency of hybridization is determined by a number of factors during hybridization and during the washing procedure, including temperature, ionic strength, length of time, and concentration of formamide. These factors are outlined in, for example, Sambrook et al. (1989), supra.

Generally, it is expected that the Herv-K/HML-2 RNA will be present in a biological sample (e.g., CSF, blood, cells, and the like) obtained from an individual at relatively low levels that may require that amplification techniques be used in detection assays. Such techniques are known in the art.

For example, the Enzo Biochemical Corporation “Bio-Bridge” system uses terminal deoxynucleotide transferase to add unmodified 3′-poly-dT-tails to a DNA probe. The poly-dT-tailed probe is hybridized to the target nucleotide sequence, and then to a biotin-modified poly-A. PCT publication WO 84/03520 and European patent application EPA124221 describe a nucleic acid hybridization assay in which: (1) analyte is annealed to a single-stranded DNA probe that is complementary to an enzyme-labeled oligonucleotide; and (2) the resulting tailed duplex is hybridized to an enzyme-labeled oligonucleotide. European patent application EPA204510 describes a DNA hybridization assay in which analyte DNA is contacted with a probe that has a tail, such as a poly-dT tail, an amplifier strand that has a sequence that hybridizes to the tail of the probe, such as a poly-A sequence, and which is capable of binding a plurality of labeled strands.

Non-PCR-based, sequence specific nucleic acid amplification techniques can also be used in the invention to detect Herv-KJHML-2 RNA. An example of such techniques include, but are not necessarily limited to, the Invader assay, see, e.g., Kwiatkowski et al. (1999) Mol. Diagn. 4:353-364. See also U.S. Pat. No. 5,846,717.

A particularly desirable technique may first involve amplification of the target Herv-K/HML-2 RNA from a sample. This may be accomplished, for example, by the polymerase chain reactions (PCR) technique described, for example, in Saiki et al. (1986) Nature 324:163-166; U.S. Pat No. 4,683,195, and U.S. Pat. No.4,683,202. Other amplification methods are well known in the art.

The probes, or alternatively nucleic acid isolated or derived from the samples, may be provided in solution for such assays, or may be affixed to a support (e.g., solid or semi-solid support). Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, and Protein A beads.

In one embodiment, the probe (or sample RNA or nucleic acid produced from the sample RNA) is provided on an array for detection. Arrays can be created by, for example, spotting polynucleotide probes onto a substrate (e.g., glass, nitrocellulose, and the like) in a two-dimensional matrix or array. The probes can be bound to the substrate by either covalent bonds or by nonspecific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Techniques for constructing arrays and methods of using these arrays are described, for example, in EP 721 016; EP 728 520; EP 785 280; EP 799 897; WO 95/22058; WO 97/29212; WO 97/27317; WO 97/02357; and U.S. Pat. Nos. 5,593,839, 5,578,832, 5,599,695, 5,556,752, 5,631,734. Arrays are particularly useful where, for example a single sample is to be analyzed for the presence of two or more nucleic acid target regions, as the probes for each of the target regions, as well as controls (both positive and negative) can be provided on a single array. Arrays thus facilitate rapid and convenience analysis.

Methods of Detecting Herv-K/HML-2 Polypeptides

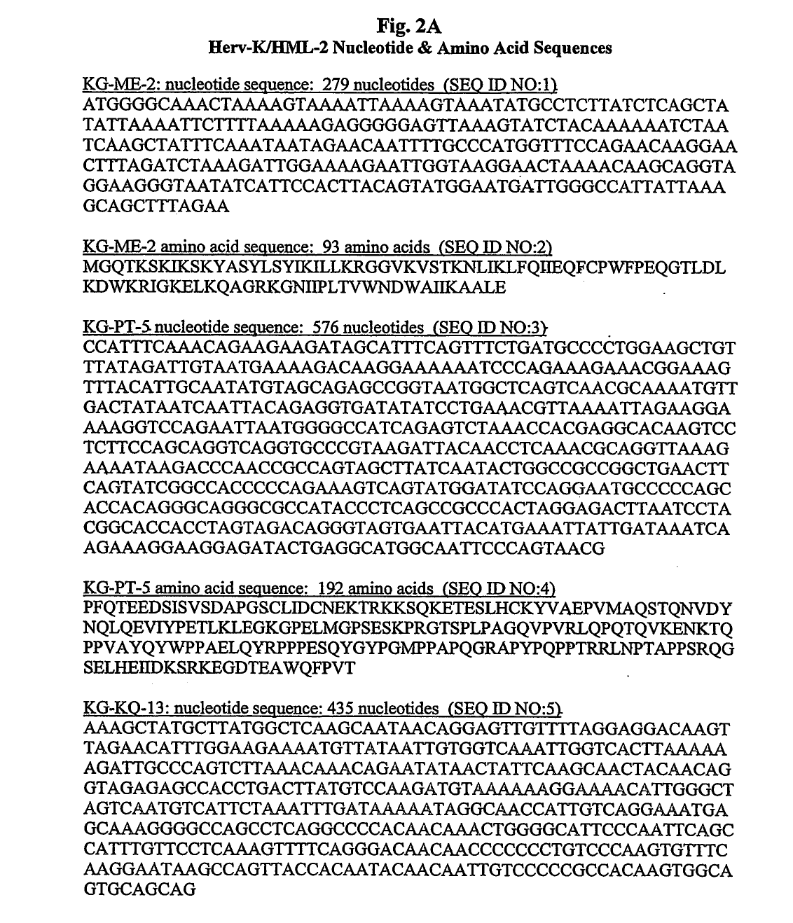

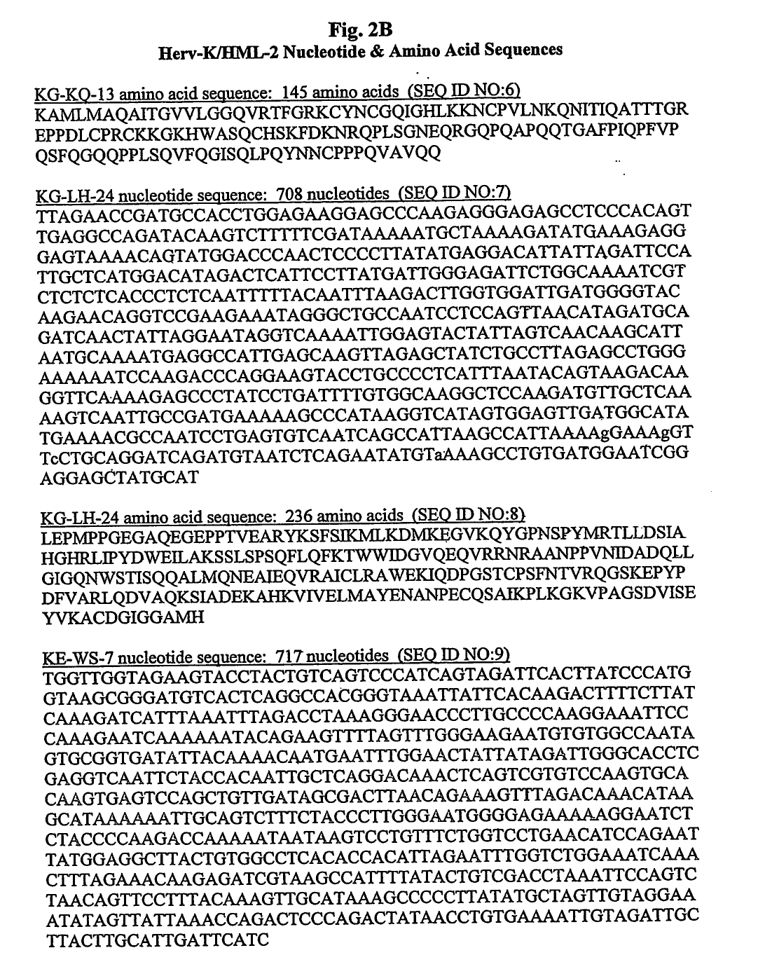

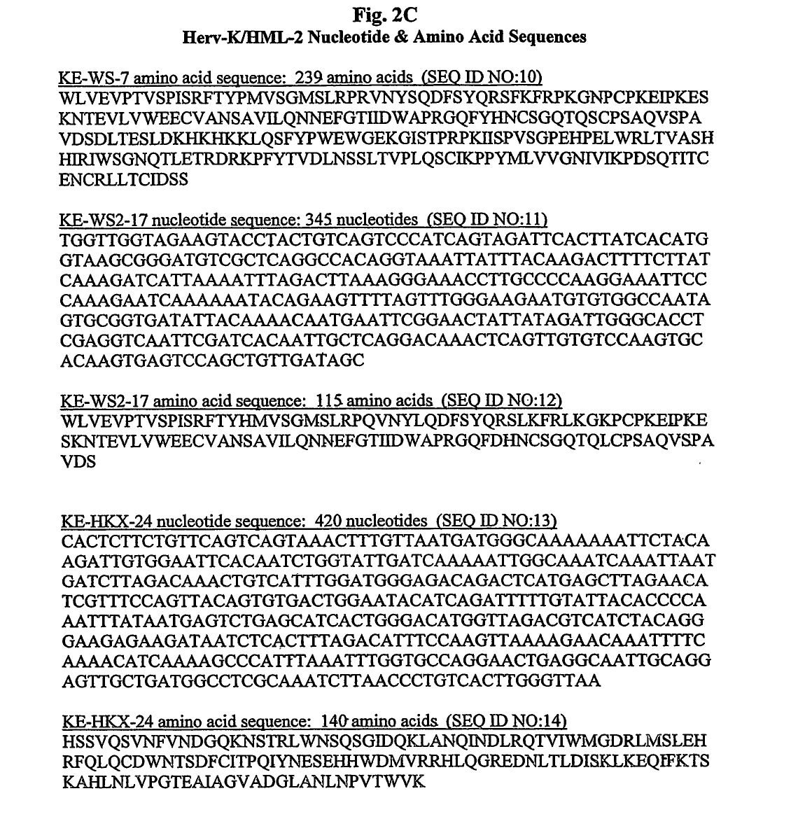

In one embodiment, the invention features methods for detecting Herv-K/HML-2 expression in a sample by detection of a Herv-K/HML-2 polypeptide in a biological sample. Of particular interest is detection of a Herv-K/HML-2 GAG polypeptide, such as that exemplified by the amino acid sequence designated KG-HE-2 in FIG. 2A-2C and that exemplified by amino acid sequence designated herein as SE-HA. Also of particular interest is detection of a Herv-K/HML-2 ENV polypeptide, those exemplified by the amino acid sequences in FIG. 2A-2C.

Polypeptide-based detection of Herv-K/HML-2 can be accomplished by use of a receptor (including ligand-binding receptor fragments) or an antibody (including antigen-binding antibody fragments) that specifically binds the target Herv-K/HML-2 polypeptide (e.g., an anti-Herv-K/HML-2 GAG polypeptide antibody). For example, the presence of Herv-K/HML-2 polypeptides in a sample can be determined using a Herv-K/HML-2-specific probe using various techniques known in the art including, but not limited to, quantitative immunoassays, such as, radioimmunoassay, immunofluorescent assay, enzyme immunoassay, chemiluminescent assay, ELISA, or western blot assay, as described in Coligan et al., eds., 1991, supra

Polypeptide-based detection of Herv-K/HML-2 can be accomplished using a variety of biological samples, e.g., blood or blood derivatives (e.g., serum, plasma, and the like), CSF, urine, cells, tissues, and the like. The anti-Herv-K/HML-2 antibody can be generated so as to detect the Herv-K/HML-2 polypeptide on a surface of a cell which expressed the polypeptide, on the surface of an Herv-K/HML-2 viral particle, or as free polypeptide (e.g., not associated with either a host cell or a viral particle, such as may be present in a sample due to lysis of the viral particle or cell which expressed the polypeptide). Anti-Herv-K/HML-2 antibodies are particularly useful reagents since generally antibodies are highly specific for the target antigen.

In one embodiment, the invention features immunoassays to determine the presence of Herv-K/HML-2 polypeptide (including Herv-K/HML-2 polypeptide present on viral particles) in a biological sample, e.g., a cell or a body fluid sample, by contacting the sample with an antibody (usually, but not necessarily, a monoclonal antibody); reacting the sample and the antibody for a time and under conditions that allow the formation of an immunocomplex between the antibody and Herv-K/HML-2 virus particles and/or Herv-K/HML-2 polypeptide in the sample; and detecting the immunocomplex. The presence of an immunocomplex indicates the presence of Herv-K/HML-2 polypeptide in the sample and, thus, indicates that Herv-K/HML-2 has been and/or is being expressed in the individual.

Design of the immunoassays is subject to a great deal of variation, and many formats are known in the art. The immunoassay will utilize at least one viral epitope derived from Herv-K/HML-2. In one embodiment, the immunoassay uses a combination of viral epitopes derived from Herv-K/HML-2. These epitopes may be derived from the same or from different viral polypeptides, and may be in separate recombinant or natural polypeptides, or together in the same recombinant polypeptides. An immunoassay may use, for example, a monoclonal antibody directed towards a viral epitope(s), a combination of monoclonal antibody directed towards a viral epitope(s), a combination of monoclonal antibodies directed towards epitopes of one viral antigen, monoclonal antibodies directed towards epitopes of different viral antigens, polyclonal antibodies directed towards the same viral antigen, or polyclonal antibodies directed towards different viral antigens.

Protocols may be based, for example, upon competition, or direct reaction, or sandwich type assays. Protocols may also, for example, use solid supports, or may be by immunoprecipitation. Most assays involve the use of labeled antibody or polypeptide; the labels may be, for example, enzymatic, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

The immunoassay may be, without limitations, in a heterogeneous or in a homogeneous format, and of a standard or competitive type. In a heterogeneous format, the anti-Herv-K/HML-2 antibody is typically bound to a solid support to facilitate separation of the sample from Herv-K/HML-2 polypeptide after incubation. After reaction for a time sufficient to allow for antibody-antigen complex formations, the solid support containing the antibody is typically washed prior to detection of bound polypeptides. Both standard and competitive formats are known in the art.

In a homogeneous format, the test sample is incubated with anti-Herv-K/HML-2 antibody in solution. For example, it may be under conditions that will precipitate any antigen-antibody complexes which are formed. Both standard and competitive formats for these assays are known in the art.

In a standard format, the level of Herv-K/HML-2 polypeptide-antibody complex is directly monitored. This may be accomplished by, for example, determining whether labeled anti-xenogeneic (e.g., anti-human) antibodies which recognize an epitope on anti-Herv-K/L-2 antibodies will bind due to complex formation. In a competitive format, the amount of Herv-K/HML-2 polypeptide in the sample is deduced by monitoring the competitive effect on the binding of a known amount of labeled Herv-K/HML-2 polypeptide (or other competing ligand) in the complex. Amounts of binding or complex formation can be determined either qualitatively or quantitatively.

Complexes formed comprising Herv-K/HML-2 polypeptide and anti-Herv-K/HML-2 antibody are detected by any of a number of known techniques, depending on the format. For example, unlabeled anti-Herv-K/HML-2 antibodies in the complex may be detected using a conjugate of anti-xenogeneic Ig complexed with a label, (e.g., an enzyme label).

The antibody in the immunoassays for detection of Herv-K/HML-2 polypeptides may be provided on a support (e.g., solid or semi-solid); alternatively, the polypeptides in the sample can be immobilized on a support. Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, and Protein A beads. Bead-based supports are generally more useful for immobilization of the antibody in the assay.

In one embodiment, the biological sample contains cells (i.e., whole cells) and detection is by reacting the sample with labeled antibodies, performed in accordance with conventional methods. In general, antibodies that specifically bind a Herv-K/HML-2 polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, and others). The absence or presence of antibody binding can be determined by various methods, including, but not limited to, flow cytometry of dissociated cells, microscopy, radiography, and scintillation counting. Any suitable alternative methods of qualitative or quantitative detection of levels or amounts of differentially expressed polypeptide can be used, for example ELISA, western blot, immunoprecipitation, radioimmunoassay, and the like.

In another embodiment of this assay, the immunocomplex can be detected by a competitive immunoassay by reacting the anti-Herv-K/HML-2 antibody with the sample and with a competing antigen to which the antibody is known to specifically bind, e.g., a detectably labeled Herv-K/HML-2 antigen or an immobilized competing antigen such as an isolated viral protein. The competing antigen can be labeled or immobilized.

Alternatively, the immunoassay is a sandwich immunoassay that uses a second antibody, e.g., a monoclonal antibody, that either also binds Herv-K/HML-2 viral polypeptides or binds to the first monoclonal antibody, one of the two antibodies being immobilized and the other being labeled using standard techniques. In the sandwich immunoassay procedures, the Herv-K/HML-2 polypeptide-binding antibody can be a capture antibody attached to an insoluble material, and the second Herv-K/HML-2 polypeptide-binding antibody can be a detector or labeling antibody.

Methods of Detecting Herv-K/HML-2 Antibodies

In another aspect, the presence of Herv-K/HML-2 expression in an individual may be detectable by assaying an appropriate biological sample from the individual for anti-Herv-K/HML-2 antibodies. In some embodiments, of particular interest is the detection of anti-Herv-K/HML-2 GAG polypeptide antibodies. In some embodiments, of interest is the detection of anti-Herv-K/HML-2 ENV polypeptide antibodies. The presence of anti-Herv-K/HML-2 antibodies in a sample can be determined by various techniques well known in the art including, but not limited to, quantitative immunoassays, such as, radioimmunoassay, immunofluorescent assay, enzyme immunoassay, chemiluminescent assay, ELISA, or western blot assay. In these assays, the biological sample contains the anti-Herv-K/HML-2 antibodies and a Herv-K/HML-2 antigen is used to detect the presence of the antibody. Exemplary methods are described, for example, in Examples 1-5.

Anti-Herv-K/HML-2 antibodies can be detected by, for example, obtaining a biological sample from an individual having or suspected of having Herv-K/HML-2 expression (e.g., suspected of having ALS), and which biological sample is suspected of containing an antibody that specifically binds to Herv-K/HML-2. The biological sample of the individual is contacted with an isolated Herv-K/HML-2 particle or with a Herv-K/HML-2 polypeptide (e.g., a Herv-K/HML-2 GAG polypeptide) or antigenic fragment thereof. Formation of antibody-viral particle or antibody-polypeptide complexes is monitored by standard techniques (see, for example, Harlow et al., 1988, supra).

Typically, an immunoassay for an anti-Herv-K/HML-2 antibody(s) will involve selecting and preparing the test sample suspected of containing the antibodies, such as a biological sample (e.g., blood or serum), then incubating it with an antigenic (e.g., epitope-containing) Herv-K/HML-2 polypeptide(s) under conditions that allow antigen-antibody complexes to form, and then detecting the formation of such complexes. Suitable incubation conditions are well known in the art.

Antibodies in the test sample that cross react with non-Herv-K/HML-2 particles or non-Herv-K/HML-2 polypeptides can be depleted from the test sample using standard control screening steps where desired. Variations on methods of detecting anti-Herv-K/HML-2 antibodies are similar to those described above for detection of Herv-K/HML-2 viral particles and/or Herv-K/HML-2 polypeptides and other variations that will be readily apparent to the ordinarily skilled artisan upon reading the present specification.

The immunoassays for detection of anti-Herv-K/HML-2 polypeptide antibodies may be conducted using an Herv-K/HML-2 polypeptide on a support (e.g., solid or semi-solid), as herein exemplified in the Example section; alternatively, the antibodies in the sample can be immobilized on a support for contacting with a Herv-K/HML-2 polypeptide. Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, and Protein A beads. Bead-based supports are generally more useful for immobilization of the Herv-K/HML-2 polypeptide in this embodiment of the invention.

In an exemplary embodiment, screening for anti-Herv-K/HML-2 antibodies in a sample is accomplished by contacting a biological sample with an isolated Herv-K/HML-2 polypeptide. An interaction between an antibody in the sample and the Herv-K/HML-2 protein is monitored by standard techniques (see, for example, Harlow et al., 1988, supra). Detection of antibody-Herv-K/HML-2 polypeptide complexes indicates that the sample contains anti-Herv-K/HML-2 antibodies, and in turn that the patient has generated a humoral response against the Herv-K/HML-2 polypeptide, which in turn indicates that Herv-K/HML-2 has been expressed or is being expressed in the individual.

### COMPOSITIONS OF THE INVENTION

Anti-Herv-K/HML-2 Antibodies

In yet another embodiment, the invention provides an antibody that specifically binds to a Herv-K/HML-2 polypeptide, which polypeptide may be associated with or separate from a Herv-K/HML-2 viral particle. The antibody can be generated using isolated, intact Herv-K/HML-2 viral particles, an antigenic portion of the virus, an isolated Herv-K/HML-2 polypeptide or an antigenic portion of an isolated Herv-K/HML-2 polypeptide. Such antibodies are generally referred to herein as anti-Herv-K/HML-2 antibodies.

In particular, the invention provides an antibody that specifically binds to a Herv-K/HML-2 GAG polypeptide. The invention also provides an antibody that specifically binds to a Herv-K/HML-2 ENV polypeptide. More particularly, the invention provides an antibody that specifically binds to the Herv-K/HML-2 GAG polypeptide KG-ME-2, or to a polypeptide comprising the amino acid sequence of KG-ME-2 (SEQ ID NO:2). Even more particularly, the invention provides an antibody that specifically binds to the Herv-K/HML-2 GAG polypeptide of about amino acid 31 to about amino acid 93 of KG-ME-2 polypeptide, or to a polypeptide comprising the amino acid residues of about amino acid 31 to about amino acid 93 of KG-ME-2.

As used herein, the term “antibody” refers to a polypeptide or group of polypeptides which are comprised of at least one antibody combining site. An “antibody combining site” or “binding domain” is formed from the folding of variable domains of an antibody molecule(s) to form three-dimensional binding spaces with an internal surface shape and charge distribution complementary to the features of an epitope of an antigen, which allows an immunological reaction with the antigen. An antibody combining site may be formed from a heavy and/or a light chain domain (V_{H} and V_{L}, respectively), which form hypervariable loops which contribute to antigen binding. The term “antibody” includes, for example, vertebrate antibodies, hybrid antibodies, chimeric antibodies, altered antibodies, univalent antibodies, the Fab proteins, and single domain antibodies.

Determination of immunogenicity of a protein and generation of an antibody to a virus or a protein are techniques well known in the art (see, for example Harlow et al., 1988, supra). By “immunogenic portion” or “immunogenically effective portion” is meant a portion of a virus or viral polypeptide, which is of sufficient size and/or conformation that when injected into an animal causes an immune response and antibodies are generated which bind to the immunogenic portion.

Methods for production of antibodies that specifically bind a selected antigen are well known in the art. Immunogens for raising antibodies can be prepared by mixing a Herv-K/HML-2 polypeptide with an adjuvant, and/or by making fusion proteins with larger immunogenic proteins. Herv-K/HML 2 polypeptides can also be covalently linked to other larger immunogenic proteins, such as keyhole limpet hemocyanin. Immunogens are typically administered intradermally, subcutaneously, or intramuscularly to experimental animals such as rabbits, sheep, and mice, to generate antibodies. Monoclonal antibodies can be generated by isolating spleen cells and fusing myeloma cells to form hybridomas.

Preparations of polyclonal and monoclonal antibodies specific for polypeptides encoded by a selected polynucleotide are made using standard methods known in the art. Typically, at least 6, 8, 10, or 12 contiguous amino acids are required to form an epitope. Epitopes that involve non-contiguous amino acids may require a longer polypeptide, e.g., at least 15, 25, or 50 amino acids. Antibodies that specifically bind to Herv-K/HML-2 polypeptides are generally those that provide a detection signal at least 5-, 10-, or 20-fold higher than a detection signal provided with non-Herv-K/HML-2 proteins when used in western blots or other immunochemical assays. Preferably, antibodies that specifically bind polypeptides of the invention do not bind to other proteins in immunochemical assays at detectable levels and can immunoprecipitate the specific polypeptide from solution.

As noted above, “antibodies” encompasses various kinds of antibodies, including, but not necessarily limited to, naturally occurring antibodies, single domain antibodies, hybrid antibodies, chimeric antibodies, single-chain antibodies, antibody fragments that retain antigen binding specificity, human antibodies, humanized antibodies, and the like.

Naturally occurring antibodies specific for Herv-K/HML-2 polypeptides, particularly for Herv-K/HML-2 GAG and/or ENV polypeptides, more particularly for Herv-K/HML-2 GAG polypeptides comprising the amino acid sequence of KG-ME-2, even more particularly for Herv-K/HML 2 GAG polypeptides comprising the amino acid sequence about amino acid 31 to about amino acid 93 of KG-ME-2 can be obtained according to methods well known in the art. For example, serum antibodies to a polypeptide of the invention in a human population can be purified by methods well known in the art, e.g., by passing antiserum over a column to which Herv-K/HML-2 viral particle, or the corresponding selected polypeptide or fusion protein is bound. The bound antibodies can then be eluted from the column, for example using a buffer with a high salt concentration.

The invention also encompasses single domain antibodies, hybrid antibodies, chimeric antibodies, single-chain antibodies, and antibody fragments that retain antigen binding specificity. As used herein, a “single domain antibody” (dAb) is an antibody which is comprised of an V_{H} domain, which reacts immunologically with a designated antigen. A dAb does not contain a V_{L} domain, but may contain other antigen binding domains known to exist in antibodies, for example, the kappa and lambda domains. Methods for preparing dAbs are known in the art. Antibodies may also be comprised of V_{H} and V_{L} domains, as well as other known antigen binding domains. Examples of these types of antibodies and methods for their preparation are known in the art, and include the following.

“Vertebrate antibodies” refers to antibodies which are tetramers or aggregates thereof, comprising light and heavy chains which are usually aggregated in a “Y” configuration and which may or may not have covalent linkages between the chains. In vertebrate antibodies, the amino acid sequences of all the chains of a particular antibody are homologous with the chains found in one antibody produced by the lymphocyte which produces that antibody in situ, or in vitro (for example, in hybridomas). Vertebrate antibodies typically include native antibodies, for example, purified polyclonal antibodies and monoclonal antibodies. Methods for the preparation of these antibodies are known in the art.

“Hybrid antibodies” are antibodies wherein one pair of heavy and light chains is homologous to those in a first antibody, while the other pair of heavy and light chains is homologous to those in a different second antibody. Typically, each of these two pairs will bind different epitopes, particularly on different antigens. This results in the property of “divalence”, i.e., the ability to bind two antigens simultaneously. Such hybrids may also be formed using chimeric chains, as set forth below.

“Chimeric antibodies”, are antibodies in which the heavy and/or light chains are fusion proteins. Typically the constant domain of the chains is from one particular species and/or class, and the variable domains are from a different species and/or class. Also included is any antibody in which either or both of the heavy or light chains are composed of combinations of sequences mimicking the sequences in antibodies of different sources, whether these sources be differing classes, or different species of origin, and whether or not the fusion point is at the variable/constant boundary. Thus, antibodies can be produced in which neither the constant nor the variable region mimic known antibody sequences, thus providing for antibodies having a variable region that has a higher specific affinity for a particular antigen, or having a constant region that can elicit enhanced complement fixation, or to make other improvements in properties possessed by a particular constant region.

The invention also encompasses “altered antibodies”, which refers to antibodies in which the naturally occurring amino acid sequence in a vertebrate antibody has been varied. Utilizing recombinant DNA techniques, antibodies can be redesigned to obtain desired characteristics. The possible variations are many, and range from the changing of one or more amino acids to the complete redesign of a region, for example, the constant region. Changes in the constant region, in general, to attain desired cellular process characteristics, e.g., changes in complement fixation, interaction with membranes, and other effector functions. Changes in the variable region may be made to alter antigen binding characteristics. The antibody may also be engineered to aid the specific delivery of a molecule or substance to a specific cell or tissue site. The desired alterations may be made by known techniques in molecular biology, e.g., recombinant techniques, site directed mutagenesis, and other techniques.

Further exemplary antibodies include “univalent antibodies”, which are aggregates comprised of a heavy chain/light chain dimer bound to the Fc (i.e., constant) region of a second heavy chain. This type of antibody escapes antigenic modulation. See, e.g., Glennie et al. (1982) Nature 295:712-714.

Included also within the definition of antibodies are “Fab” fragments of antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab′), as well as tetramers containing the 2H and 2L chains (referred to as F(ab)₂), which are capable of selectively reacting with a designated antigen or antigen family. “Fab” antibodies may be divided into subsets analogous to those described above, i.e., “vertebrate Fab”, “hybrid Fab”, “chimeric Fab”, and “altered Fab”. Methods of producing “Fab” fragments of antibodies are known within the art and include, for example, proteolysis, and synthesis by recombinant techniques.

Herv-K/HML-2 Nucleic Acid

In one aspect, the invention features polynucleotides of Herv-K/HML-2. “Herv-K/HML-2 polynucleotides” as used herein generally refers to polynucleotides that can be used to specifically identify Herv-K/HML-2 expression (e.g., as in a nucleic acid probe in detection by hybridization) are of particular interest. Exemplary of such polynucleotides are those having at least a portion of a sequence of the gag gene of Herv-K/HML-2, which sequence is useful in specific detection of Herv-K/HML-2 RNA expression, for example, in a biological sample from an individual with ALS. Exemplary Herv-K/HML-2 gag polynucleotide sequences encompassed by the invention include, but are not necessarily limited to, sequences of KG-ME-2, KG-PT-5, KG-LH24 and KG-KQ-13 as described in FIGS. 2A-2C and Examples 1 and 2. Exemplary polynucleotides of the invention thus also encompass those having, as a contiguous sequence, a sequence immediately 5′ of the Herv-K/HML-2 GAG-encoding sequence (e.g., a GAG open reading frame (ORF)) and a sequence within a 5′ portion of the Herv-K HML-2 GAG-encoding region are also contemplated by the invention. Likewise, exemplary polynucleotides of the invention include polynucleotides having, as a contiguous sequence, a sequence within a 3′ portion of the Herv-K/HML-2 GAG-encoding region and a sequence immediately 3′ of the Herv-K/HML-2 GAG-encoding region.

Other specific, exemplary Herv-K/HML-2 polynucleotides contemplated by the invention are those polynucleotides that encode a Herv-K/HML-2 GAG polypeptide, including, for example, the polypeptides of KG-ME-2, KG-PT-S, KG-LH24 and KG-KQ-13 as described in FIGS. 2A-2C, as well as polynucleotide that specifically hybridizes to such a polynucleotide molecule or a portion thereof. A Herv-K/HML-2 polynucleotide of particular interest is one comprising a sequence encoding a polypeptide having an amino acid sequence of the Herv-K/HML-2 GAG polypeptide designated KG-ME-2, e.g., a polypeptide having at least the amino acid sequence of the contiguous amino acid residues about 1 to about 93 of KG-ME-2 amino acid sequence. Another Herv-K/HML-2 polynucleotide of particular interest is one comprising a sequence encoding a polypeptide having an amino acid sequence of the Herv-K/HML-2 GAG polypeptide designated KG-ME-2, e.g., a polypeptide having at least the amino acid sequence of the contiguous amino acid residues about 31 to about 93 of KG-ME-2 amino acid sequence, e.g., at least about 1-4 contiguous amino acid residues from amino acid residues about 31 to about 93 of KG-ME-2 amino acid sequence, at least about 2-5 contiguous amino acid residues from amino acid residues about 31 to about 93 of KG-ME-2 amino acid sequence, at least about 4-10 contiguous amino acid residues from amino acid residues about 31 to about 93 of KG-ME-2 amino acid sequence, at least about 8-15 contiguous amino acid residues from amino acid residues about 31 to about 93 of KG-ME-2 amino acid sequence, at least about 12-20 contiguous amino acid residues from amino acid residues about 31 to about 93 of KG-ME-2 amino acid sequence, at least about 20-40 contiguous amino acid residues from amino acid residues about 31 to about 93 of KG-ME-2 amino acid sequence, up to 63 contiguous amino acid residues from amino acid residues about 31 to about 93 of KG-ME-2 amino acid sequence. Further specific exemplary Herv-K/HML-2 polynucleotides include polynucleotides having at least about 10 contiguous nucleotides, at least about 15 contiguous nucleotides, at least about 20 contiguous nucleotides, at least about 50 contiguous nucleotides of KG-ME-2 nucleotide sequence.

Other specific, exemplary Herv-K/HML-2 polynucleotides of the invention are those having at least a portion of a sequence of the env gene of Herv-K/HML-2, which sequence is useful in specific detection of Herv-K/HML-2 RNA expression, for example, in a biological sample from an individual with ALS. Exemplary Herv-K/HML-2 env polynucleotide sequences encompassed by the invention include, but are not necessarily limited to, sequences of KE-WS-7, KE-WS2-17 and KE-HKX-24 as described in FIGS. 2A-2C and Examples 1 and 2. Exemplary polynucleotides of the invention thus also encompass those having, as a contiguous sequence, a sequence immediately 5′ of the Herv-K/HML-2 ENV-encoding sequence (e.g., an ENV ORF) and a sequence within a 5′ portion of the Herv-K/HML-2 ENV-encoding region are also contemplated by the invention. Likewise, exemplary polynucleotides of the invention include polynucleotides having, as a contiguous sequence, a sequence within a 3′ portion of the Herv-K/HML-2 ENV-encoding region and a sequence immediately 3′ of the Herv-K/HML-2 ENV-encoding region. Other specific, exemplary Herv-K/HML-2 polynucleotides contemplated by the invention are those polynucleotides that encode a Herv-K/HML-2 ENV polypeptide, including, for example, the polypeptides of KE-WS-7, KE-WS2-17 and KE-HKX-24 as described in FIGS. 2A-2C, as well as polynucleotide that specifically hybridizes to such a polynucleotide molecule or a portion thereof.

The invention also encompasses polynucleotides having sequence complementary to the sequence of the polynucleotides described herein; RNA having a sequence corresponding to DNA sequences described herein; viral genes corresponding to the provided polynucleotides; polynucleotides obtained from the biological materials described herein or other biological sources (particularly human sources) (e.g., by hybridization under stringent conditions, particularly conditions of high stringency); variants of the provided polynucleotides and their corresponding genes, particularly those variants that are present due to the degeneracy of the genetic code (referred to herein as “degenerate variants”) and other variants that are specific to Herv-K/HML-2 sequences of the invention or retain a biological activity of the gene product encoded by a polynucleotide specifically described herein (e.g., retain the biological activity of the GAG polypeptide in, for example, its reactivity of Herv-K/HML-2 GAG-specific antibodies). Other nucleic acid compositions contemplated by and within the scope of the present invention will be readily apparent to one of ordinary skill in the art when provided with the disclosure here.

The polynucleotides of the subject invention can be isolated and obtained in substantial purity, generally as other than an intact chromosome or intact viral particle. Usually, the polynucleotides, either as DNA or RNA, will be obtained substantially free of other naturally-occurring nucleic acid sequences, generally being at least about 50%, usually at least about 90% pure and can be “recombinant”, e.g., flanked by one or more nucleotides with which it is not normally associated on a naturally occurring chromosome, as exemplified herein.

The polynucleotides of the invention can be provided as a linear molecule or within a circular molecule, and can be provided within autonomously replicating molecules (vectors) or within molecules without replication sequences. Expression of the polynucleotides can be regulated by their own or by other regulatory sequences known in the art. The polynucleotides of the invention can be introduced into suitable host cells using a variety of techniques available in the art, such as polycation-mediated DNA transfer, transfection with naked or encapsulated nucleic acids, liposome-mediated DNA transfer, intracellular transportation of DNA coated latex beads, protoplast fusion, viral infection, electroporation, gene gun, calcium phosphate-mediated transfection, and the like.

The host cells suitable for use in production of recombinant host cells can be any prokaryotic or eukaryotic cell suitable for, for example, maintenance and/or replication of vectors containing Herv-K/HML-2 nucleic acid, or for replication and production of Herv-K/HML-2 viral particles. Exemplary host cells include, but are not necessarily limited to, bacterial, yeast, and mammalian host cells. Isolated recombinant host cells containing Herv-K/HML-2 nucleic acid are also contemplated by the invention. Isolated recombinant vectors or constructs containing Herv-K/HML-2 nucleic acid are likewise contemplated by the invention. Such vectors can include other components for expression of polypeptides encoded by the Herv-K/HML-2 nucleic acid (e.g., promoter elements, transcription termination elements, enhancers, and the like), as well as element for the maintenance, replication, or (optionally) genomic integration of the construct in the host cell (e.g., origin of replication, and the like).

The isolated Herv-K/HML-2 polynucleotides of the invention can be provided with 5′, 3′ or both 5′ and 3′ flanking sequences. Suitable flanking sequences include, but are not necessarily limited to, promoter sequence, enhancer sequences, transcriptional start and/or stop sites, construct or vector sequences (e.g., sequences that provide for manipulation of the polynucleotide within a linear or circular molecule (e.g., plasmid), including, but not necessarily limited to, sequences for replication and maintenance of the construct or vector, sequences encoding gene products that provide for selection (e.g., antibiotic resistance or sensitivity, factors that affect growth in media with or without supplements, and the like), sequences that provide for production or a fusion protein with the polynucleotide and a heterologous polypeptide (i.e., a polypeptide encoded by a polynucleotide that originates from a source other than the polynucleotide to which it is operably linked), and the like.

The polynucleotides of the invention include polynucleotides having sequence similarity or sequence identity. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under stringent conditions, for example, at 50° C. or higher and 0.1×SSC (9 mM saline/0.9 mM sodium citrate).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 2000 nucleotides, preferably less than about 1000 nucleotides in length, more preferably less than about 500 nucleotides, less than about 200, 150, 100, 75, 50 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1.0 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60° C. to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and % is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity.

For example, if sequences with up to and including about 90% identity are sought, the Tm can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ.

Using the equation, hybridization and wash compositions, and desired Tₘ, those of ordinary skill will understand that variations in the stringency of hybridization and/or wash solutions are within the scope of the present disclosure, as are variations in the lengths of the hybridization and wash steps (e.g., from minutes (e.g., 15 min to 30 min) to hours (e.g., 1-2 hrs to overnight). If the desired degree of mismatching results in a Tₘ of less than 45° C. (aqueous solution) or 32° C. (formamide solution), it is preferred to increase these concentration so that a higher temperature can be used. An extensive guide to the hybridization of nucleic acids is found in Tijssen (1993) Laboratory Techniques in Biochemistry and Molecular Biolog—Hybridization with Nucleic Acid Probes, Part I, Chapter 2 (Elsevier, New York); and Ausubel et al. (1995), supra, and Sambrook et al. (1989), supra.

Nucleic acids of particular interest are those that are substantially identical to the provided polynucleotide sequences (for example, KG-ME-2, KG-PT-5, KG-LH-24, KG-KQ-13, KE-WS2-17, KE-WS-7 and KE-HKX-24) including for example, genetically altered versions of the gene, and the like. Nucleic acids that hybridize to the provided polynucleotide sequences under stringent hybridization conditions are also of particular interest. Nucleic acid probes, particularly labeled probes of DNA sequences, can be used to isolate homologous or related Herv-K/HML-2 polynucleotides. The source of homologous nucleic acid can be any species, e.g. primate species, particularly human.

Generally, nucleic acid hybridization is performed using at least 15 contiguous nucleotides (nt) of a polynucleotide provided herein. Nucleic acid probes of at least 15 contiguous nt preferentially hybridize with a nucleic acid comprising the complementary sequence, allowing the detection, identification and retrieval of the nucleic acids that uniquely hybridize to the selected probe. Probes of more than 15 nucleotides can be used, e.g., probes of from about 18 nucleotides to about 100 nucleotides in length, but 15 nucleotides represents sufficient sequence for unique identification.

Sequence similarity and sequence identity can also be determined by sequence analysis. In general, sequence identity is calculated based on a reference sequence, which may be a subset of a larger sequence, such as a conserved motif, coding region, flanking region, and the like. A reference sequence will usually be at least about 18 contiguous nt long, more usually at least about 30 nt long, and may extend to the complete sequence that is being compared. Algorithms for sequence analysis are known in the art, such as gapped BLAST, described in Altschul et al. Nucleic Acids Res. (1997) 25:3389-3402. Sequence analysis can be performed using the Smith-Waterman homology search algorithm as implemented in MPSRCH program (Oxford Molecular). For the purposes of this invention, a preferred method of calculating percent identity is determined by the Smith-Waterman homology search algorithm as implemented in MPSRCH program (Oxford Molecular) using an affine gap search with the following search parameters: gap open penalty, 12; and gap extension penalty, 1.

Another embodiment of the invention provides an isolated polynucleotide having at least 90%, at least 92%, at least 94%, at least 96 %, at least 98%, or at least 99% sequence identity with the polynucleotides of the invention as described herein. One embodiment provides an isolated polynucleotide having at least 90%, at least 92%, at least 94%, at least 96 %, at least 98%, or at least 99% sequence identity with the sequences shown in FIG. 2A-2C. In other embodiments, isolated polynucleotides additionally have less than 85%, 83%, 80%, 75%, 70% sequence identity with the sequence of KG-ME-2 nucleotide sequence.

The nucleic acids of the invention can be cDNAs or isolated as a component of a genomic DNA (e.g., from a patient isolate), as well as fragments thereof, particularly fragments that are useful in the methods disclosed herein (e.g., in diagnosis, as a unique identifier of Herv-K/HML-2 nucleic acid, and the like). The term “cDNA” as used herein . is intended to include all nucleic acids that share an arrangement of sequence elements that can found in a native mature MRNA species, including splice variants.

The nucleic acid compositions of the invention can encode all or a part of a Herv-K/HML-2 polypeptide, e.g., Herv-K/HML-2 GAG polypeptide or Herv-K/HML-2 ENV polypeptide, or can be flanking sequences of the Herv-K/HML-2-polypeptide-encoding region. Double or single stranded fragments can be obtained from the DNA sequence by chemically synthesizing oligonucleotides in accordance with conventional methods, by restriction enzyme digestion, by PCR amplification, and the like. Isolated polynucleotides and polynucleotide fragments of the invention comprise at least about 10, about 15, about 20, about 35, about 50, about 100, about 150 to about 200, about 250 to about 300, or about 350 contiguous nucleotides selected from the polynucleotide sequences designated as KG-ME-2, KG-PT-5, KG-LH-24, KG-KQ-13, KE-WS2-17, KE-WS-7 and KE-HKX-24 (see FIG. 2A-2C). In general, fragments will be of at least 15 nucleotides, usually at least 18 nucleotides or 25 nucleotides, and up to at least about 50 contiguous nucleotides in length or more. Nucleic acid fragments of particular interest include a polynucleotide of about 279 contiguous nucleotides, and fragments thereof, and corresponding to a PCR product of a Herv-K/HML-2 GAG gene designated KG-ME-2.

The subject nucleic acid compositions can be used as single- or double-stranded probes or primers for the detection of Herv-K/HML-2 RNA or cDNA generated from such RNA, as obtained may be present in a biological sample (e.g., extracts of human cells). The Herv-K/HML-2 polynucleotides of the invention can also be used to generate additional copies of the polynucleotides, to generate antisense oligonucleotides, and as triple-strand forming oligonucleotides.

The polynucleotides of the invention, particularly where used as a probe in a diagnostic assay, can be detectably labeled. Exemplary detectable labels include, but are not limited to, radiolabels, fluorochromes, (e.g. fluoresceinisothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-F AM) or N,N,N′,N′-tetramethyl-6-carboxyrhodamine (TAMRA), radioactive labels, (e.g. ³²P, ³⁵S, and ³H), and the like. The detectable label can involve two stage systems (e.g., biotin-avidin, hapten-anti-hapten antibody, and the like). The invention also includes solid substrates such as arrays comprising any of the polynucleotides described herein. An array may have one or more different polynucleotides. The polynucleotides are immobilized on the arrays using methods known in the art.

Herv-K/HML-2 Polypeptides

The polypeptides of the invention include those encoded by the disclosed Herv-K/HML-2 polynucleotides, as well as nucleic acids that, by virtue of the degeneracy of the genetic code, are not identical in sequence to the disclosed polynucleotides but encode the same polypeptide. Of particular interest is the Herv-K/HML-2 GAG polypeptide, and fragments thereof, such as that provided in KG-ME-2, KG-PT-5, KG-LH-24, KG-KQ-13 amino acid sequences in FIG. 2A-2C, as well as in some instances, variants of such polypeptides, e.g., a polypeptide having the sequence of KG-ME-2 amino acid sequence, but with conservative amino acid substitutions. Also of interest is the Herv-K/HML-2 ENV polypeptide, and fragments thereof, such as that provided in KE-WS2-17, KE-WS-7 and KE-HKX-24 amino acid sequences in FIG. 2A-2C.

By “Herv-K/HML-2 polypeptide” is generally meant a polypeptide that can be obtained from a Herv-K/HML-2 nucleotide sequence, particularly a polypeptide that can be the basis for specific detection of expression of Herv-K/HML-2. A Herv-K/HML-2 polypeptide also can mean a polypeptide that can be obtained from a Herv-K/HML-2 viral particle. Exemplary Herv-K/HML-2 polypeptides of particular interest that is specific for Herv-K/HML-2 is a Herv-K/HML-2 GAG polypeptide, e.g., the polypeptide of KG-ME-2 amino acid sequence and fragments thereof. A Herv-K/HML-2 polypeptide of particular interest is a polypeptide having an amino acid sequence of a 5′ portion of a Herv-K/HML-2 GAG polypeptide, for example, a polypeptide having at least the amino acid sequence of the contiguous amino acid residues about 31 to about 93 of KG-ME-2 amino acid sequence, a polypeptide having at least the amino acid sequence of the contiguous amino acid residues about 21 to about 93 of KG-ME-2 amino acid sequence, a polypeptide having at least the amino acid sequence of the contiguous amino acid residues about 11 to about 93 of KG-ME-2 amino acid sequence, a polypeptide having at least the amino acid sequence of the contiguous amino acid residues about 1 to about 93 of KG-ME-2 amino acid sequence, as well as polypeptides containing such regions.

In general, the Herv-K/HML-2 polypeptides of the subject invention are separated from their naturally occurring environment. In certain embodiments, the subject protein is present in a composition that is enriched for the protein as compared to a control. As such, purified polypeptide is provided, where “purified” generally means that the protein is present in a composition that is substantially free of non-differentially expressed polypeptides, where by substantially free is meant that less than 90%, usually less than 60% and more usually less than 50% of the composition is made up of non-differentially expressed polypeptides.

The Herv-K/HML-2 polypeptides of the invention include variants of the naturally occurring Herv-K/HML-2 protein, where such variants are homologous or substantially similar to the naturally occurring protein, and can be of an origin of the same or different species as the Herv-K/HML-2 described herein (e.g., human, murine, or some other species that naturally expresses the recited polypeptide, usually a mammalian species). However, for use in methods of the invention, any variant Herv-K/HML-2 polypeptide must able to function similarly to the non-variant polypeptides. For example, for use in a method of detection of Herv-K/HML-2 expression that involves detection of anti-Herv-K/HML-2 antibodies in sera, a variant Herv-K/HML-2 must be able to bind to the anti-Herv-K/HML-2 antibodies present in the sera. In general, variant polypeptides have a sequence that has at least about 80%, usually at least about 90%, and more usually at least about 98% sequence identity with a differentially expressed polypeptide of the invention, as measured by BLAST 2.0 using the parameters described above. The variant polypeptides can be naturally or non-naturally glycosylated, i.e., the polypeptide has a glycosylation pattern, if any, that differs from the glycosylation pattern found in the corresponding naturally occurring protein. Variants of polypeptides include mutants. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted.

The Herv-K/HML-2 polypeptides of the invention also include fragments and fusion proteins having an amino acid sequence of an Herv-K/HML-2 polypeptide or a fragment thereof. Of particular interest is a Herv-K/HML-2 polypeptide fragment that is specific for Herv-K/HML-2 GAG polypeptide fragment having an amino acid sequence of KG-ME-2 amino acid sequence, for example a polypeptide having at least the amino acid sequence of the contiguous amino acid residues about 31 to about 93 of KG-ME-2 amino acid sequence.

The Herv-K/HML-2 polypeptide fragments are also encompassed by the present invention, particular antigenically effective polypeptide fragments, as well as fragments defining an epitope that can be bound by an antibody that is specific for the Herv-K/HML-2 polypeptide, for example, as found in the polypeptide containing a portion of the KG-ME-2 amino acid sequence from about amino acid 31 to about amino acid 93. A polypeptide is “antigenically effective” where the polypeptide is effective, either alone or in combination with a carrier protein, to elicit production of antibodies that specifically bind the polypeptide. Thus, Herv-K/HML-2 polypeptides and polypeptide fragments of the invention can be used as a vaccine. As used herein, “epitope” refers to an antigenic determinant of a polypeptide. An epitope can comprise about 3 or more amino acids in a spatial conformation which is unique to the epitope. Generally an epitope consists of at least about 5 such amino acids, and more usually, consists of at least about 8-10 such amino acids. Some epitopes comprise more than 10 amino acids and may involve the structure of the polypeptide. Methods of determining the spatial conformation of amino acids are known in the art, and include, for example, x-ray crystallography and 2-dimensional nuclear magnetic resonance.

A polypeptide is “antigenically reactive” with an antibody when it binds to an antibody due to antibody recognition of a specific epitope contained within the polypeptide. Antigenic reactivity may be determined by antibody binding, more particularly by the kinetics of antibody binding, and/or by competition in binding using as competitor(s) a known polypeptide(s) containing an epitope against which the antibody is directed. The techniques for determining whether a polypeptide is antigenically reactive with an antibody are known in the art.

Polypeptide fragments of interest will typically be at least about 10 amino acids, at least about 15 amino acids, usually at least about 20 amino acids, at least about 50 amino acids, at least about 55 amino acids, at least about 60 to about 63 amino acids in length and can be as long as I 00 amino acids in length or longer.

As discussed in more detail in the Examples below, the portion of the Herv-K/HML-2 GAG polypeptide designated KG-ME-2 is an epitope that can serve as a specific marker for the expression of Herv-K/HML-2 in a biological sample, particularly in a biological sample from an individual with ALS.

Pharmaceutical Compositions

The invention further contemplates pharmaceutical compositions comprising at least one of a Herv-K/HML-2 polypeptide or a Herv-K/HML-2 polynucleotide, which is provided in a pharmaceutically acceptable excipient. In particular, pharmaceutical compositions comprise at least one of a Herv-K/HML-2 GAG polypeptide or a Herv-K/HML-2 GAG-encoding polynucleotide. Preferably, the Herv-K/HML-2 GAG polypeptide in the pharmaceutical composition comprises a polypeptide comprising the amino acid sequence of KG-ME-2 or a fragment thereof. The pharmaceutical composition comprising a Herv-K/HML-2 polynucleotide preferably comprises a polynucleotide that encodes the amino acid sequence of KG-ME-2 or a fragment thereof. In some instances, pharmaceutical compositions comprise at least one of a Herv-K/HML-2 ENV polypeptide or a Herv-K/HML-2 ENV-encoding polynucleotide.

Pharmaceutical compositions comprising a Herv-K/HML-2 polypeptide or a Herv-K/HML-2 polynucleotide may be used, for example, to generate an immune response against the polypeptide or the encoded polypeptide and/or against the Herv-K/HML-2 virus, and thus can be used, for example, in a vaccine. Such an immune response may include a humoral and/or cellular immune response, including a T cell immune response. Accordingly, such a generated immune response would help to decrease the spread of a viral infection and/or ameliorate a symptom of a Herv-K/HML-2-associated disease, such as ALS.

As used herein, “pharmaceutically acceptable excipient” includes any material which, when combined with an active ingredient of a composition, allows the ingredient to retain biological activity and without causing disruptive reactions with the subject's immune system. Various pharmaceutically acceptable excipients are well known in the art.

Exemplary pharmaceutically carriers include sterile aqueous of non-aqueous solutions, suspensions, and emulsions. Examples include, but are not limited to, any of the standard pharmaceutical excipients such as a phosphate buffered saline solution, water, emulsions such as oil-water emulsion, and various types of wetting agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition of a Herv-K/HML-2 polypeptide or Herv-K/HML-2 polynucleotide may also be lyophilized using means well known in the art, for subsequent reconstitution and use according to the invention. Also of interest are formulations for liposomal delivery and formulations comprising microencapsulated Herv-K/HML-2 polypeptides or Herv-K/HML-2 polynucleotides. Compositions comprising such excipients are formulated by well known conventional methods (see: for example, Remington's Pharmaceutical Sciences, 18th Ed., Mack Publishing Co.).

In general, the pharmaceutical compositions can be prepared in various forms, such as granules, tablets, pills, suppositories, capsules (e.g. adapted for oral delivery), microbeads, microspheres, liposomes, suspensions, salves, lotions and the like. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Stabilizing agents, wetting and emulsifying agents, salts for varying the osmotic pressure or buffers for securing an adequate pH value.

Kits

Reagents specific for detection of Herv-K/HML-2 expression, such as, for example Herv-K/HML-2 polynucleotides, Herv-K/HML-2 polypeptides, and/or anti-Herv-K/HML-2 antibodies, can be supplied in a kit for detecting the presence or absence of Herv-K/HML-2 expression in a biological sample. Such reagents can include, for example, nucleotide probes or primers for detection of Herv-K/HML-2 RNA, anti-Herv-K/HML-2 antibodies for detection of Herv-K/HML-2 viral particles and/or polypeptides, and Herv-K/HML-2 polypeptides for detection of anti-Herv-K/HML-2 antibodies in the sample. In particular, the kits can include such reagents specific for detection of Herv-K/HML-2 GAG polypeptide expression, including reagents that specifically detect expression of a polypeptide comprising a portion of the Herv-K/HML-2 GAG polypeptide designated by the KG-ME-2 sequence. The reagents can be provided in labeled vials. The kit can also include buffers or labeling components, as well as instructions for using the reagents to detect (either qualitatively or quantitatively) the target nucleic acid, polypeptide, or antibody in the biological sample. The kit can further include appropriate positive controls, negative controls, or both.

For example, nucleic acid probes can be packaged into diagnostic kits. Diagnostic kits can include one or more polynucleotide probes (e.g., DNA or RNA) which may be labeled; alternatively, the polynucleotide probe may be unlabeled and the ingredients for labeling may be included in the kit in separate containers. The kit may also contain other suitably packaged reagents and materials needed for the particular hybridization protocol, for example, standards, as well as instructions for conducting the test.

Kits suitable for immunodiagnosis and containing the appropriate labeled reagents are constructed by packaging the appropriate materials, including the polypeptides of the invention containing Herv-K/HML-2 epitope(s) or antibodies directed against Herv-K/HML-2 epitope(s) in suitable containers, along with the remaining reagents and materials required for the conduct of the assay, as well as a suitable set of assay instructions. Assays using the kits may be performed in vitro and cell-free (e.g., in vitro binding assays) or may be cell-based.

Kits suitable for vaccines and containing the appropriate labeled reagents are constructed by packaging the appropriate materials, including the polypeptides of the invention containing Herv-K/HML-2 epitope(s) in suitable containers, along with the remaining reagents and materials required for the vaccine, as well as a suitable set of vaccination instructions. The vaccine kit may or may not include adjuvants and/or pharmaceutical excipients for administration.

The following Examples are provided to illustrate, but not limit, the invention.

### EXAMPLES

### Example 1

### Assays for Detection of an Immune Response to Herv/HML Antigens

In order to test for an immune response to Herv/HML antigens in ALS patients, selected portions of various Herv/HML genes were amplified using PCR, the amplification products cloned into expression plasmids and recombinant Herv/HML polypeptides expressed in bacteria. The resultant recombinant polypeptides were then subjected to gel electrophoresis and western blot analysis using standard techniques as described herein. Primary antibody used as a probe for some of the western blots was sera from individuals with ALS or sera from non-ALS individuals (e.g., blood donors).

To generate the specific Herv/HML polynucleotide sequences, primers were constructed based on particular Herv/HML gag gene and env gene sequences and used to amplify human genomic DNA (HGD). For example, selected portions of the Herv-K/HML-2 gag and env genes were amplified using the sequence of the endogenous retrovirus HervK-109/Herv-K10 as the staring point for the design of oligonucleotide primers. The HervK-109 DNA sequence is found at GenBank accession number AF164615 and the Herv-K10 DNA sequence is found at GenBank accession number M14123. The sequences of the primers used and the viral genes to which the primers were directed are provided in Table 1. PCR was carried out according to methods well known in the art, generally using the Expand High Fidelity PCR System (Roche Diagnostics, Cat. No. 1732641). Specifically, a DNA template sample (for example, human genomic DNA (1 mg/ml; Clontech, Cat. No. 6550-1) or plasmid DNA (about 25 ng)) in 10 μl was mixed with 10 μl 10× buffer, 2 μl 10 mM nNTP, 4 μl 20 μM Primer 1, 4 μl 20 μM Primer 2, 1.5 μl of 3.5 u/μl Taq DNA Polymerase and 68.5 μl water. The PCR cycles were as follows: 1 min. at 95° C.; 35 cycles of 15 sec. at 94° C., 30 sec. at 55-60° C., 30-60 sec. at 68° C.; 8 min. at 68° C. The PCR products were purified using a Qiagen PCR purification kit ( Qiagen Cat. No. 28104).

TABLE 1
Primer Guide

| | | | | | |
|---|---|---|---|---|---|
| Clone | Virus | Gene | Temp* | Primer⁺ | Sequence (5′ -> 3′) |

| | | | | | |
|---|---|---|---|---|---|
| KG-ME-2 | HML-2 | gag | HGD | KG-M1F | GCGGAATTCCTCGAGATGGGGCAAACTAAAA |
| | | | | | GTAA SEQ ID NO:15 |
| | | | | KG-E93R | CTGTCGACGCGGCCGCGTTCTAAAGCTGCTT |
| | | | | | TAATAATGG SEQ ID NO:16 |
| KG-PT-5 | HML-2 | gag | HGD | KG-P94F | GCGGAATTCCTCGAGCCATTTCAAACAGAAG |
| | | | | | AAGAT SEQ ID NO:17 |
| | | | | KG-T285R | CTGTCGACGCGGCCGCACGTTACTGGGAATT |
| | | | | | GCCATGC SEQ ID NO:18 |
| KG-LH- | HML-2 | gag | HGD | KG-L286F | GCGGAATTCCTCGAGTTAGAACCGATGCCAC |
| 24 | | | | | CTGGA SEQ ID NO:19 |
| | | | | KG-H521R | CTGTCGACGCGGCCGCTATGCATAGCTCCTC |
| | | | | | CGATTCC SEQ ID NO:20 |
| KG-KQ- | HML-2 | gag | HGD | KG-522F | GAGGAATTCCTCGAGAAAGCTATGCTTATGG |
| 13 | | | | | CTCAA SEQ ID NO:21 |
| | | | | KG-Q666R | CTGTCGACGCGGCCGCACTGCTGCACTGCCA |
| | | | | | CTTGTGG SEQ ID NO:22 |
| KE-WS-7 | HML-2 | env | HGD | KE-W178- | GCGGAATTCCTCGAGTGGTTGGTAGAAGTAC |
| | | | | F | CTA SEQ ID NO:23 |
| | | | | KE-S415R | CTGTCGACGCGGCCGCGATGAATCAATGCAA |
| | | | | | GTAAGCA SEQ ID NO:24 |
| KE-WS2- | HML-2 | env | HGD | KE-W178F | GCGGAATTCCTCGAGTGGTTGGTAGAAGTAC |
| 17 | | | | | CTA SEQ ID NO:25 |
| | | | | KE-S292R | CTGTCGACGCGGCCGCCGCTATCAACAGCTG |
| | | | | | GACTCAC SEQ ID NO:26 |
| KE-HKX- | HML-2 | env | HGD | KE-H491F | GCGGAATTCCTCGAGCACTCTTCTGTTCAGT |
| 24 | | | | | CA SEQ ID NO:27 |
| | | | | KE-K630R | CTGTCGACGCGGCCGCTTAACCCAAGTGACA |
| | | | | | GG SEQ ID NO:28 |
| XJWE-1 | Herv-W | env | HGD | WE-M1-F | GCGGAATTCCTCGAGATGGCCCTCCCTTATC |
| | | | | | ATATT SEQ ID NO: 29 |
| | | | | WE-L192- | TCGGTCGACTGCGGCCGCAGGGGGAGGCATA |
| | | | | R | TCCAACAG SEQ ID NO:30 |
| XJWE-2 | Herv-W | env | HGD | WE-G119- | GCGCAATTCCTCGAGGGAGTTCAAGATCAGG |
| | | | | F | CA SEQ ID NO:31 |
| | | | | WE-R317- | TCGGTCGACTGCGGCCGCTCTCTTTTGTTGC |
| | | | | R | GGGGCTTAG SEQ ID NO:32 |
| XJWE-3 | Herv-W | env | HGD | WE-F346- | GCGCAATTCCTCGAGTTCTACTACAAACTAT |
| | | | | F | CTCA SEQ ID NO:33 |
| | | | | WE-W444- | TCGGTCGACTGCGGCCGCCATTGGCTGAGGA |
| | | | | R | GGCCCCAGG SEQ ID NO:34 |
| JW-A | Herv-W | gag | HGD | WG-M1 F | GCGGAATTCCTCGAGATGTTCTCCACCCTCC |
| | | | | | CCAA SEQ ID NO:35 |
| | | | | WG-C151 | CTGTCGACGCGGCCGCGGCATAATTGGGGAA |
| | | | | R | TATTGGC SEQ ID NO:36 |
| JW-D | Herv-W | gag | HGD | WG-Q131 | GCGGAATTCCTCGAGCAAAAGGAGATAGACA |
| | | | | F | AAAGG SEQ ID NO:37 |
| | | | | WG-G257 | CTGTCGACGCGGCCGCCTGTGGGGAATGTTT |
| | | | | R | CTCT SEQ ID NO:38 |
| JW-G | Herv-W | gag | HGD | WG-L98 F | GCGGAATTCCTCGAGTTATGCCCTACAGGAA |
| | | | | | GC SEQ ID NO:39 |
| | | | | WG-G200 | CTGTCGACGCGGCCGCGTCCTAACCCTTGTA |
| | | | | R | AAAC SEQ ID NO:40 |
| JW-H | Herv-W | gag | HGD | WG-F233 | GCGGAATTCCTCGAGTTTGGCGATCTCTGGT |
| | | | | F | ATCTC SEQ ID NO:41 |
| | | | | WG-L318 | CTGTCGACGCGGCCGCCCAGAAAGGCAGTAG |
| | | | | R | GATT SEQ ID NO:42 |
| JW-1 | Herv-W | gag | HGD | WG-M302 | GCGGAATTCCTCGAGATGATGTCCACCATAA |
| | | | | F | CA SEQ ID NO:43 |
| | | | | WG-A352 | CTGTCGACGCGGCCGCCTGCAGCTGACTGAG |
| | | | | R | TGAT SEQ ID NO:44 |
| HML-1 | HML-1 | gag | HGD | HK-1 F | CGCGAATTCCTCGAGATGGGACAAAGTGAAA |
| JR | | | | | GC SEQ ID NO:45 |
| | | | | HK-1 R | CTGTCGACGCGGCCGCGCTCAAGAGCTGCCT |
| | | | | | TTAT SEQ ID NO:46 |
| HML-4 | HML-4 | gag | HGD | HK4-F | GCGGAATTCCTCGAGATGGGACAAGCCAGTA |
| XJ | | | | | CA SEQ ID NO:47 |
| | | | | HK4-R | CTGTCGACGCGGCCGCATTCTAAGATGGAGC |
| | | | | | GAAC SEQ ID NO:48 |
| HML-5 | HML-5 | gag | HGD | HK5-F | GCGGAATTCCTCGAGATGGGACAACAGTTAT |
| XJ | | | | | CA SEQ ID NO:49 |
| | | | | HK5-R | CTGTCGACGCGGCCGCGGGCCAGAGCTGCCC |
| | | | | | TAAC SEQ ID NO:50 |
| HML-6 | HML-6 | gag | HGD | HK6-F | GCGGAATTCCTCGAGATGTGCAGTTGCTTAG |
| XJ | | | | | AG SEQ ID NO:51 |
| | | | | HK6-R | CTGTCGACGCGGCCGCGCAGAAGTACAGTAT |
| | | | | | GAAC SEQ ID NO:52 |
| XKG-11 | HML-2 | gag | HGD | KG-K11-F | GCGGAATTCAAATATGCCTCTTATCTCAGCT |
| | | | | | SEQ ID NO:53 |
| | | | | KG-E93-R | CTGTCGACGCGGCCGCGTTCTAAAGCTGCTT |
| | | | | | TAATAAT SEQ ID NO:54 |
| XKG-21 | HML-2 | gag | HGD | KG-121-F | GCGGAATTCATTCTTTTAAAAAGAGGGGGA |
| | | | | | SEQ ID NO:55 |
| | | | | KG-E93-R | CTGTCGACGCGGCCGCGTTCTAAAGCTGCTT |
| | | | | | TAATAAT SEQ ID NO:56 |
| XKG-31 | HML-2 | gag | HGD | KG-S31-F | GCGGAATTCTGTACAAAAAATCTAATCAAG |
| | | | | | SEQ ID NO:57 |
| | | | | KG-E93-R | CTGTCGACGCGGCCGCGTTCTAAAGCTGCTT |
| | | | | | TAATAAT SEQ ID NO:58 |
| XKG-45 | HML-2 | gag | HGD | KG-F45-F | GCGGAATTCWTTTGCCCWTGGTTYCCA |
| | | | | | SEQ ID NO:59 |
| | | | | KG-S103- | CTGTCGACGCGGCCGCCTGAAACACTATCTT |
| | | | | R | CTTCTGT SEQ ID NO:60 |
| X31-83 | HML-2 | gag | KG- | KG-S31-F | GCGGAATTCTCTACAAAAAATCTAATCAAG |
| | | | ME-2 | | SEQ ID NO:61 |
| | | | | KG-N83-R | CTGTCGACGCGGCCGCGTTCTAAAGCTGCTT |
| | | | | | TAATAAT SEQ ID NO:62 |
| X31-73 | HML-2 | gag | KG- | KG-S31-F | GCGGAATTCTCTACAAAAAATCTAATCAAG |
| | | | ME-2 | | SEQ ID NO:63 |
| | | | | KG-K73-R | CTGTCGACGCGGCCGCCCTTCCTACCTGCTT |
| | | | | | GTTTTAGT SEQ ID NO:64 |
| X31-63 | HML-2 | gag | KG- | KG-S31-F | GCGGAATTCTCTACAAAAAATCTAATCAAG |
| | | | ME-2 | | SEQ ID NO:65 |
| | | | | KG-I63-R | CTGTCGACGCGGCCGCCAATTCTTTTCCAAT |
| | | | | | CTTTTAGA SEQ ID NO:66 |
| XKG-1- | HML-2 | gag | HGD | KG-M1-F | GCGGAATTCCTCGAGATGGGGCAAACTAAAA |
| 53 | | | | | GTAA SEQ ID NO:67 |
| | | | | KG-G53-R | CTGTCGACGCGGCCGCTTCCTTGTTCTGGAA |
| | | | | | ACCATGG SEQ ID NO:68 |
| JR-1-83 | HML-2 | gag | KG- | KG-M1-F | GCGGAATTCCTCGAGATGGGGCAAACTAAAA |
| | | | ME-2 | | GTAA SEQ ID NO:69 |
| | | | | KG-N83 R | GCGGCCGCATTCCATACTGTAAGTGGAATGA |
| | | | | | TA SEQ ID NO:70 |
| KG-ME- | HML-2 | gag | KG- | HisLink-3 | P-TCGACCCATCACCATCACCATCATTGCA |
| 2.9 | | | ME-2 | | SEQ ID NO:71 |
| | | | | HisLink-4 | P-ATGATGGTGATGGTGATGGG |
| | | | | | SEQ ID NO:72 |

*KG-ME-2 = plasmid of that particular clone
⁻P - = phosphate group; F = Forward; R = Reverse

The plasmid pThiohis-A from the His-Patch ThioFusion Expression System (Invitrogen, Cat. No. K350-01) was used for cloning the amplified DNA. The purified PCR product and the plasmid vector were subjected to restriction endonuclease digestion with EcoRI and NotI (New England Biolabs). The restriction enzyme digestion products were purified using a Qiagen PCR purification kit and ligated using T4 DNA ligase (Promega, Cat. No. M1801) and incubating at 16° C. for 16 hours in standard conditions. Following incubation, the ligation reaction was used to transform competent E. coli bacteria (One Shot® TOP 10 Chemically Competent E. coli, Invitrogen, Cat. No. C4040-10) using standard procedures. Resultant colonies were screened for the presence of the desired insert using PCR with the same set of primers used to generate the insert. The identity of the cloned DNA insert was confirmed by DNA sequencing.

The recombinant protein SE-HA (amino acids 31 to 93 of KG-ME-2) was generated via ligation of a 6 histidine linker into the Pst I and Sal I sites of the vector pThiohis-A. This was followed by excision of the thioredoxin sequences via digestion of KG-ME-2 plasmid with NdeI and EcoRI. The thioredoxin sequences were replaced with an oligonucleotide duplex containing the hemaglutinnin (HA) epitope (YPYDVPDYA, SEQ ID NO:73). Deletion of the thioredoxin sequences and in-frame insertion of the HHHHHH and HA epitope sequences was verified with the use of monoclonal antibodies to thioredoxin (InVitrogen, Carlsbad Calif.), HHHHH (Qiagen, Valencia, Calif.), and the HA epitope (Roche Diagnostics, Indianapolis, Ind.). New oligonucleotide primers were then employed to amplify amino acids 31 to 93 of Herv-K/HML-2 gag sequence, which was introduced into the EcoRI and NotI sites of the modified vector. Detection of insert containing clones was performed as described above. When produced and isolated, both the KG-ME-2 and the SE-HA antigen were purified on ProBond resin (InVitrogen) according to manufacturer's instructions.

To generate recombinant Herv/HML polypeptide, bacteria containing the recombinant plasmid were grown and induced to express the cloned Herv/HML DNA by the addition of IPTG (from 0.1 to 5 mM) to the bacterial growth media and incubation for an additional 2 to 3 hours at 37° C. The cells in 1.5 ml of the IPTG-treated bacterial culture were collected by centrifugation. After resuspension in 100 μl PBS, the bacteria were lysed by the addition of 100 μl 2× denaturing protein gel sample buffer. The bacterial cell lysated was heated at 95° C. for 5 minutes and then 10-20 μl of the preparation was loaded into a 4-12% Bis-Tris polyacrylamide gel (Invitrogen). The gel was run at about 120 mV for about 1 hour in MOPS or MES Running Buffer (Invitrogen) until the proteins were separated over the length of the gel.

Using standard western blotting techniques, the separated polypeptides were transferred from the gel to a nitrocellulose membrane (Schleicher and Schuell/VWR) using an InVitrogen XCELL module transfer apparatus at about 25 mV per 1-3 gels for about 1.5 hours. After blocking the membrane in BLOTTO (150 mM NaCl, 20 mM Tris, pH 7.5, 0.1% Tween-20, 2.5% (volume/volume) normal goat sera, 2.5% (weight/volume) Carnation non fat dry milk) in for 30 minutes to overnight at 4° C., the membranes were washed in TBS (150 mM NaCl, 20 mM Tris pH 7.5) and reacted with the primary antibody at room temperature overnight with gentle agitation. When sera was used as source of the primary antibody, the sera was typically diluted 1:100 in BLOTTO plus 0.02% sodium azide. The sera was typically preadsorbed to reduce background reactivity to bacterial proteins. The preadsorption was performed by incubation of the diluted serum overnight with a nitrocellulose filter disc that had been immersed in a diluted solution whole E. coli cell lysate proteins. When a monoclonal antibody was used as the primary antibody, the monoclonal antibody was diluted as recommended by the manufacturer and was typically used at concentrations of 1-10 μg/ml. After incubation with the primary antibody, the membranes were washed twice with TBS, 5 minutes each, and then incubated with the secondary antibody in BLOTTO for 1 hour at room temperature. After incubation with the secondary antibody, the membrane was washed four times with TBS, 5 minutes each with gentle agitation. The secondary antibody used was typically labeled with alkaline phosphatase and detected using SigmaFast (5)-Bromo-4Chloro-3-Indolyl Phosphate/Nitro Blue Tetrazolium (Sigma Chemical) as the substrate. After the blots were dry, immunoreactive bands were quantitated using a scanner and appropriate software.

### Example 2

### Detection of an Immunologic Response to Herv-K/HML-2 Antigens

To test for expression of Herv/HML in ALS patients, sera from individuals with ALS was screened for the presence of anti-Herv/HML antigen antibodies. For this analysis, selected portions of the Herv/HML genes of interest were amplified, cloned into a pThioHisA vector, expressed in bacteria as thioredoxin-fusion or HA epitope-fusion proteins and subjected to western blot analysis as described in Example 1.

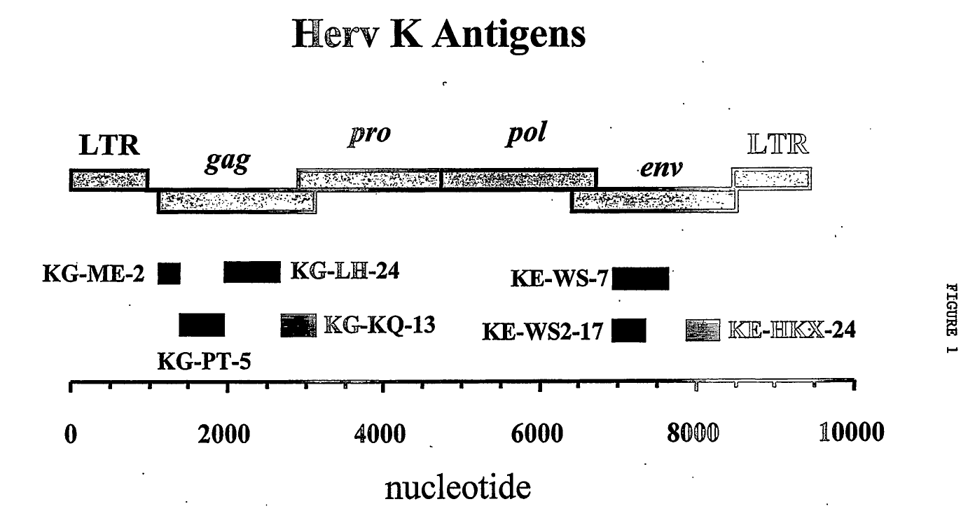

Accordingly, selected portions of the Herv-K/HML-2 gag and env genes were amplified by PCR (FIG. 1) using sequences of HervK-109/Herv-K10 as the starting point for the design of oligonucleotide primers as described in Example 1 and Table 1. The amplified products were then treated as described in Example 1. Confirmation of the desired cloned fragment by DNA sequencing indicated that, overall, the clones were >95% homologous to the appropriate region of Herv-K-109 (GenBank accession number AF164615) or Herv-K10 (GenBank accession number M14123). Nucleotide and amino acid sequences of the 7 Herv-K/HML-2 GAG or ENV polynucleotides and polypeptides generated (KG-ME-2, KG-PT-5, KG-KQ-13, KG-LH-24, KE-WS-7, KE-WS2-17 and KE-HKX-24) are presented in FIG. 2A-C.

Whole cell lysates from bacteria expressing the recombinant viral antigens were analyzed by western blot analysis using sera from individuals with ALS. Western blots were also performed using sera from blood donors (non-ALS individuals) as a control for the ALS sera and, to confirm the presence of a significant amount and the appropriate size of recombinant protein on the blots, a monoclonal antibody to the thioredoxin portion of the fusion protein diluted 1:5000 (InVitrogen; catalog # R920-25). Goat anti-human IgG alkaline phosphatase conjugated antibody was used as the secondary antibody to detect serum antibodies bound to the blot.

Results from screening sera from ALS and non-ALS individuals are presented in Table 2 as the number of sera positive over the total number of sera tested. The results demonstrate that individuals with ALS exhibit immunoreactivity to GAG and/or ENV sequences from Herv-K/HML-2.

TABLE 2
Seroprevalence to Herv-K/HML-2 GAG
and ENV recombinant proteins

| | Herv K GAG | | | | Herv K ENV | | |
|---|---|---|---|---|---|---|---|
| | KG- | KG- | KG- | KG- | KE- | KE- | KE- |
| | ME-2 | PT-5 | LH-24 | KQ-13 | WS-7 | WS2-17 | HKX-24 |
| ALS | 14/21 | 5/18 | 5/16 | 1/13 | 3/11 | 6/21 | 2/19 |
| | 67% | 28% | 31% | 8% | 27% | 29% | 11% |
| non- | 6/25 | 2/11 | 0/3 | 0/4 | 3/10 | 4/18 | 0/7 |
| ALS | 24% | 18% | | | 30% | 22% | |

As can be seen in Table 2, most of the ALS individuals have sera that is reactive to the Herv-K/HML-2 GAG polypeptide KG-ME-2. Fully 67% of individuals with ALS have IgG reactivity with KG-ME-2 as compared to 24% of non-ALS blood donors (a statistically significant difference, p <0.007). The next most reactive antigens were KE-WS2-17/KE-WS-7 (the two clones have significant overlap, see FIG. 1), KG-PT-5, and KG-LH-24, which reacted with 27-31% of sera from both types of individuals—with ALS and non-ALS blood donors. The two antigens from the 3′ end of the gag and env proteins, KG-KQ-13 and KE-HKX-24 exhibited very low percentage of immunoreactivity with ALS sera and with sera from non-ALS blood donors.

The results presented in Table 2 represent the presence of IgG antibodies in the tested sera since the secondary antibody used in these assays was a anti-human IgG antibody. Sera from ALS and non-ALS individuals were also tested for an IgM antibody response to KG-ME-2 and KE-WS2-17 with western blots using a goat anti-human IgM alkaline phosphatase conjugated antibody (Kirkegaard & Perry) as the secondary antibody.

The IgG and IgM reactivity of 21 ALS sera with KG-ME-2 and KE-WS2-17 is shown in Table 3. In Table 3, + indicates reactive sera, − indicates non-reactive sera and nd indicates that the analysis was not done.

TABLE 3
Immunoreactivity of individual ALS
sera with Herv-K/HML-2 antigens.

| | |
|---|---|
| KG-ME-2 | KE-WS2-17 |

| ALS sera | IgG | IgM | IgG | IgM |
|---|---|---|---|---|
| 1 | + | + | − | − |
| 2 | − | − | + | − |
| 3 | + | − | − | − |
| 4 | − | − | − | − |
| 5 | − | − | − | − |
| 6 | − | − | + | − |
| 7 | − | + | − | + |
| 8 | + | − | − | − |
| 9 | + | − | + | − |
| 10 | + | − | − | − |
| 11 | + | − | + | + |
| 12 | + | − | − | − |
| 13 | + | − | − | − |
| 14 | + | − | − | − |
| 15 | + | − | − | − |
| 16 | + | + | − | − |
| 17 | − | + | − | + |
| 18 | − | − | − | − |
| 19 | + | − | + | − |
| 20 | + | − | + | − |
| B | + | nd | − | nd |
| Positive | 14 (67%) | 4 (20%) | 6 (29%) | 3 (15%) |

As shown in Table 3, overall 4 of the 20 sera tested had an IgM response to KG-ME-2 and 3 of the 20 sera had an IgM response to KE-WS2-17. An IgM response is consistent with and may indicate a relatively recent exposure of the individual to the antigen.

Taking into account immunoreactivity to GAG and ENV proteins, 16 of 21 (76%) had an IgG response to one or more Herv-K proteins. Taking into account both IgG and IgM reactivity, 18 of 21 (86%) individuals with ALS had an antibody response to one or more Herv-K/HML-2 proteins. One of the 3 individuals with no detectable antibody response to Herv-K/HML-2, ALSA4, was the only patient in this panel diagnosed with the familial form of ALS. Thus reactivity to Herv-K/HML-2 viral proteins is highly prevalent in sporadic ALS.

Herv-K/HML-2 related sequences have been isolated from an individual with mantle cell lymphoma Therefore, sera samples from individuals with lymphoma were examined for immunoreactivity of towards KG-ME-2 and KE-WS2-17. Immunoreactivity to KG-ME-2 and KE-WS2-17 in sera from non-ALS blood donors was tested as a control. The results are shown in Table 4. In Table 4, nd indicates that analysis was not done.

TABLE 4
IgG and IgM immunoreactivity of KG-ME-2 and KE-WS2-17

| | |
|---|---|
| KG-ME-2 | KE-WS2-17 |

| | IgG | IgM | IgG | IgM |
|---|---|---|---|---|
| ALS | 14/21 | 4/20 | 6/21 | 3/20 |
| | 67% | 20% | 29% | 15% |
| Lymphoma | 2/11 | nd | 1/11 | nd |
| | 18% | | 9% | |
| non-ALS Blood | 6/25 | 0/17 | 4/18 | 1/17 |
| Donors | 24% | | 22% | 6% |

As shown in Table 4, the majority of individuals with lymphoma did not have a detectable immunoreactivity to these Herv-K/HML-2 proteins. The difference between the percentage of ALS individuals with IgG reactivity to KG-ME-2 (67%) and the percentage of individuals with lymphoma with IgG reactivity to KG-ME-2 (18%) is statistically significant (p<0.03).

Testing of 17 non-ALS blood donors for an IgM response to KG-ME-2 or KE-WS2-17 found only one individual with an IgM response to KE-WS2-17. This was compared to IgM immunoreactivity to KG-ME-2 in 4 of the 20 ALS individuals and to KE-WS2-17 in 3 of the 20 ALS individuals. Thus, IgM antibody responses to Herv-K/HML-2 proteins are more prevalent in ALS patients than in non-ALS blood donors.

### Example 3

### Expanded Study for Immunologic Response to Herv-K/HML-2 GAG Antigen

Plasma from 37 patients with sporadic ALS was collected over a period of 18 months. The patients were diagnosed by El Escorial criteria (Brooks et al. (1994) J. Neurol. Sci. 124(suppl):96-107) at the Forbes Norris MDA/ALS Research Center (San Francisco, Calif.) and had blood drawn in accordance with the California Pacific Medical Center and University of California San Francisco (UCSF) committees on human research guidelines, coordinated by the UCSF AIDS and Cancer Specimen Resource program. Clinical status of patients was evaluated using the Revised-ALS Functional Rating Scale (ALSFRS-R), scored 0-48 (The ALS CNTF treatment study (ACTS) phase I-II Study Group, The Amyotrophic Lateral Sclerosis Functional Rating Scale (1996) Arch Neurol. 53:141-147). Patients were evaluated within a month of donating samples. Control sera included 19 plasma samples from patients with Alzheimer's disease (AD). Healthy controls consisted of 80 plasma samples obtained from blood donors from the Stanford University blood bank. Plasma and lymphocytes from ALS patient blood was obtained via percoll gradient centrifugation of 15 mls of whole blood. The supernatant fraction (above the lymphocyte layer) was retained and frozen at −70° C. until use. Plasma was obtained from blood donors via centrifugation of whole blood collected in yellow-top anticoagulant tubes.

The patients consisted of 26 men and 11 women who had been diagnosed with ALS for 4 to 93 months. The median ALSFRS-R score of the cohort was 33 with a range of 8 to 43 (normal=48). Previous neurological conditions in the ALS patients included 2 cases of polio, one patient whose maternal grandmother had a mild dementia and one patient whose father had Parkinson's disease. The majority of the patients (31 of 37) were undergoing therapy with Riluzole and 12 patients were using various anti-inflammatory medications (e.g., Celebrex, Vioxx, Naproxyn, Excedrin). Five of the patients also had a second aliquot of plasma obtained between 3 to 14 months after the initial sample was obtained.

To evaluate the immunoreactivity of patients with ALS towards Herv-K/HML-2, recombinant fusion proteins expressing the 5′ gag sequences of Herv-K/HML-2 were produced as described in Example 1. The larger of the recombinant proteins, KG-ME-2, fused E. coli thioredoxin to amino acids 1 to 93 of Herv-K/HML-2 gag precursor protein followed by a 6 histidine tail near its carboxy terminus. The smaller recombinant protein, SE-HA, contained the hemaglutirnn (HA) epitope tag fused to amino acids 31-93 of the Herv-K/HML-2 gag polyprotein also with a 6 histidine tail near the carboxy terminus. Both KG-ME-2 and SE-HA polypeptides were purified via immobilized metal-ion affinity chromatography using standard methods.

The integrity and reactivity of the purified recombinant proteins was verified by western blot analysis. KG-ME-2 protein was co-electrophoresed with non-recombinant thioredoxin to differentiate reactivity to the GAG insert from immunoreactivity with thioredoxin. When the blots were incubated with a monoclonal antibody to the sequence HHHHH, non-recombinant thioredoxin and the KG-ME-2 and SE-HA proteins were all clearly visualized. The higher molecular weight bands also visualized are derived from multimeric forms of each of the three proteins. Incubation of a duplicate blot with sera from a non-ALS blood donor revealed an immunoreactive contaminating protein with a molecular weight of ˜31 kdal in the SE-HA protein preparation, but no reactivity with thioredoxin, KG-ME-2 or SE-HA. Incubation of a duplicate blot with sera from an individual with ALS results in visualization of the KG-ME-2 and SE-HA recombinant proteins, including multimers, but not non-recombinant thioredoxin.

The reactivity of purified SE-HA protein with the entire sera panel from individuals with ALS was determined by ELISA. For the ELISA, 96 well nickel-nitrilotriacetic acid NiNTA) microtiter plates (Qiagen) were incubated with 100 μl/well of a 2.5 μg/ml solution of purified SE-HA protein. After one hour at 37° C., the solutions were aspirated, the wells washed once with TBS and blocked as described above. Wells were washed one time with TBS and 100 μl of test serum diluted to an IgG concentration of 100 μg/ml (equivalent to a dilution of ˜1:120) in BLOTTO was added to duplicate wells. Monoclonal antibody controls were diluted as recommended by manufacturer. Sera and controls were incubated with antigen for 1.5 hours at room temperature with gentle rocking at which time the sera was aspirated from the wells, and the wells were washed three times with TBS. Then, 100 μl of 1:5000 diluted anti-human IgG or anti mouse alkaline phosphatase conjugate was added and any bound antibody was detected as described above. All sera were tested in at least 2 separate assays. Results obtained with a pilot group of healthy blood donors were employed to set a cut-off for positivity at an average OD for all assays equal to or greater than 0.7. Statistical analyses of all assays and clinical parameters were performed employing the programs Excel (Microsoft, Redmond Wash.), Prism, or InStat (Graph Pad Software, San Diego, Calif.).

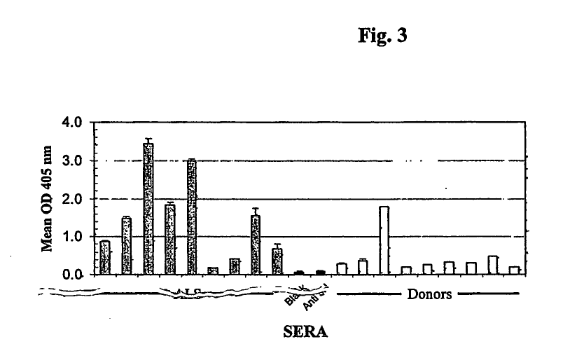

Results from a representative ELISA assay are presented in FIG. 3. In this assay only 1 of 9 non-ALS blood donors exhibited significant reactivity (O.D.>0.7) compared to 6 of 9 individuals with ALS. Nor was any reactivity seen with a monoclonal antibody to the poly-His tails of the antigens, since the poly-His sequence was bound to the nickel on the plate. Results from ELISA testing of the entire panel are presented in Table 5.

TABLE 5
Reactivity of sera from various groups with the SE-HA antigen

| Group | SE-HA Reactive | Negative |
|---|---|---|
| Sporadic ALS | 21 (57%) * † | 16 (43%) |
| AD | 3 (16%) * | 16 (84%) |
| Healthy | 8 (10%) † | 72 (90%) |

* Significantly different than AD samples. P < 0.01, Fisher's exact test.
† Significantly different than healthy donors. P < 0.001, Fisher's exact test.

As shown in Table 5, overall, 21 of 37 (57%) patients with sporadic ALS were reactive with SE-HA antigen. This compared with sera from 8 of 80 non-ALS blood donors (10%, p<0.0001) and 3 of 19 individuals (16%, p<0.005) with early stage Alzheimer's disease (AD). The reactivity rates of AD patients and healthy blood donors (16% vs 10%) with the SE-HA antigen were not statistically distinguishable (p=0.44). Thus individuals with ALS have a significantly increased incidence of IgG reactivity towards Herv-K gag sequences as compared to blood donors or individuals with Alzheimer's disease.

The elevated reactivity to SE-HA could reflect active production and immunological recognition of Herv-K/HML-2 viral particles or it could reflect a long-lived IgG response to an event that occurred long before the advent of neurological disease. One way of distinguishing between these two possibilities would be to look for IgM reactivity to Herv-K/HML-2 gag, since an IgM response would indicate a recent immune response. Therefore, sera from individuals with ALS were re-tested for an IgM antibody response to KG-ME-2. As before, murine (IgG) monoclonal antibody to thioredoxin verified expression of KG-ME-2. The two sera shown had strong IgM reactivity to KG-ME-2 but not thioredoxin. No reactivity to KG-ME-2 was seen with a serum from a non-ALS blood donor. Comparison of the reactivity obtained to electrophoresed human IgG and IgM confirmed that the anti IgM-alkaline phosphatase conjugate was specific for IgM.

Overall, 4 of the 37 ALS sera (1 1%) tested positive for IgM reactivity to Herv-K/HML-2 in contrast to none of 30 non-ALS blood donors. Additionally, one of the five individuals from whom duplicate samples were available developed IgG reactivity to SE-HA antigen in the second sample. The other four patients did not exhibit positive reactivity to SE-HA in either sample. Thus some individuals with ALS do have a significant IgM response to Herv-K/HML-2 and are in the process of seroconverting to Herv-K/HML-2.

The presence of a high rate of antibody reactivity to Herv-K/HML-2 proteins in sporadic ALS patients implies that these individuals have been recently exposed to Herv-K/HML-2 viral proteins.

### Example 4

### Assay for an Immunoreactivity to Other Endogenous Retroviral Antigens

Herv-K/HML-2 is only one subfamily of a greater group of type B/mouse mammary tumor virus (MMTV) related, endogenous retroviruses that are found in the human genome (Medstrand et al. (1993)). Given the high incidence of immunoreactivity to Herv-K/HML-2 proteins in ALS individuals, assays were performed to look for evidence of immunoreactivity to antigens from other endogenous retroviruses.

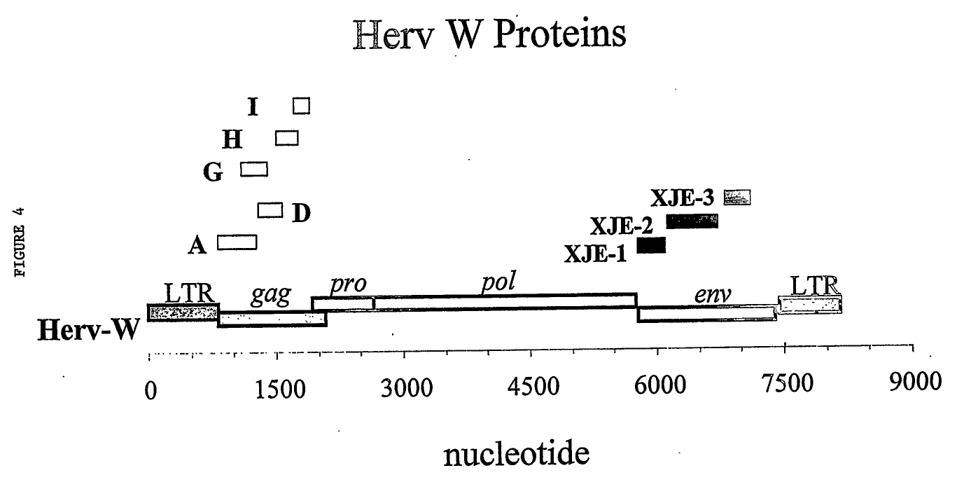

Multiple regions of the GAG and ENV proteins of Herv-W were amplified by PCR and cloned into a pThioHisA vector as described in Example 1. The regions of Herv-W amplified are depicted in FIG. 4. DNA of the resulting clones was sequenced and the cloned fragments used in the experiments were found to be homologous to the appropriate regions of Herv-W. Whole cell lysates from bacteria expressing recombinant Herv-W proteins were analyzed by western blot analysis using sera from individuals with ALS. Western blots were also performed using sera from individuals with lymphoma, sera from blood donors (non-ALS individuals) as a control for the ALS sera and, to confirm the presence of a significant amount and the appropriate size of recombinant protein on the blot, a monoclonal antibody to the thioredoxin portion of the fusion protein. Goat anti-human IgG aline phosphatase conjugated antibody was used as the secondary antibody to detect serum antibodies bound to the blot.

The results from testing the series of Herv-W proteins are presented in Table 6. In Table 6, nd indicates that analysis was not done.

TABLE 6
Reactivity of various sera with Herv-W polypeptides

| | Herv W GAG | | | | | Herv W ENV | | |
|---|---|---|---|---|---|---|---|---|
| | JW-A | JW-D | JW-G | JW-H | JW-I | XJE-1 | XJE-2 | XJE-3 |
| ALS | 0/15 | 0/11 | 0/12 | 0/12 | 0/12 | 0/10 | 0/10 | 0/10 |
| Lymphoma | nd | nd | 0/6 | 0/6 | 0/6 | 0/11 | 0/11 | 0/11 |
| Non-ALS | 0/11 | 0/9 | 0/9 | 1/9 | 3/9 | 0/6 | 0/6 | 0/6 |
| Blood Donors | | | | | | | | |

As indicated in Table 6, no immunoreactivity was seen for any of the Herv-W proteins with sera from individuals either with ALS or with lymphoma. Sera from some of the non-ALS blood donors was immunoreactive with recombinant proteins containing the 3′ portions of the Herv-W GAG protein. The remaining GAG and ENV proteins were also non-reactive with sera from non-ALS blood donors. Thus, although both Herv-W and Herv-K transcription is reported as up-regulated by monocyte/macrophage activation (Johnston et al. (2001) Ann. Neurol. 50:434442), individuals with ALS develop an immune response that is specific for Herv-K/HML-2.

In order to examine whether production of Herv-K particles and development of an antibody response is a consequence of the disease or a cause, the expression of Herv-K and related viruses in activated monocytes was investigated.

Peripheral blood mononuclear cells (PBMCs) were obtained from a healthy individual and cultured overnight. The next day the attached cells (primarily monocytes/macrophages and granulocytes) and unattached cells (primarily T and B cells) were separated and total RNA prepared. The RNA was then subject to RT-PCR using retroviral pol region consensus primers. The PCR products obtained were then hybridized to seven different probes corresponding to Herv-K/HML-2 and six HML-2 related viruses (HML-1, 3, 4, 5, 6, and Herv-K C4) previously described (Medstrand et al. (1993); Mayer et al. (2002) Genomics 80:331-343; Seifarth et al. (1998) J. Virol. 72:8384-8391; Medstrand et al. (1997) J. Gen. Virol. 78:1731-1744; Tassabehji et al. (1994) Nuc. Acids Res. 22:5211-5217. Each of these viruses is between 64 to 78% homologous to Herv-K/HML-2 in the reverse transcriptase gene region amplified. Negative controls included probes for human T cell leukemia virus (HTLV)-1 and 2 and mouse mammary tumor virus (MMTV). To control for differences in cell number, the RNA was also amplified using primers specific for glyceraldhyde-3-phosphate dehydrogenase (GAPDH) and histone H3.

This assay was based on a protocol described in Seifarth et al. (2000) AIDS Res. Hum. Retrovirus. 16:721-729. PBMCs obtained from a healthy blood donor were put into culture at 37° C. overnight. The next day the media and any unattached cells were removed and centrifuged at 1500× g. Attached cells were then washed once with PBS, harvested and pelleted by centrifugation. Both the attached and unattached cells were then washed an additional time with PBS and total RNA was prepared from both samples using a commercially available kit (RNAeasy, Qiagen). After a 2 hour incubation at 37° C. with RNAse-free DNAse (Roche Diagnostics), DNAse was removed by phenol-chloroform extraction and ethanol precipitation. The RNA was then resuspended in distilled water and aliquots subject to reverse transcription using the Titan coupled reverse transcription (RT) PCR kit (Roche Diagnostics) with 250 μM dNTPs supplemented with 12.5 μM digoxigenin dUTP, 5 mM DTT, 10 units RNAsin (Promega, Madison, Wis.), and oligonucleotide primers BDF 5′-GAAGGATCCTGGAMD GTiYTDCCHCARGG (SEQ ID NO:74) and BDR 5′-GTCGGATCCiWDAT RTCATCMATRTA (SEQ ID NO:75), where i=inosine. To control for DNA contamination, each RNA sample was also PCR amplified in the absence of RT. Duplicate aliquots of RNA were subjected to RT-PCR using control primers homologous for GAPDH (5′-CGGAGTCAACGG ATTTGGTCG (SEQ ID NO:76) and 5′-AGCCTTCTCCATG GTGGTGAAGAC (SEQ ID NO:77); Johnston et al. (2001)) and primers homologous to histone H3 (5′-CCCTCTACTGGAGGGGTGAAGAA (SEQ ID NO:78) and 5′-CTTGCC TCCTGCAAAGCACCGAT (SEQ ID NO:79); Medstrand et al. (1992) J. Gen. Virol. 73:2463-2466).

Reverse transcription reaction occurred for 45 minutes at 42° C. followed by denaturation for 4 minutes at 94° C. The cDNA was then amplified for 35 cycles of 94° C. for 1 minute, 52° C. for 1 minute, and 72° C. for 2 minutes followed by extension at 72° C. for 8 minutes. The amplified products were then diluted in hybridization buffer (5×SSC, 5× Denhardt's solution, 10 mM EDTA, 0.5% SDS, 100 μg/ml salmon sperm DNA, pH 8.0) and denatured for 10 minutes at 90° C. Aliquots of the denatured PCR products were then applied to streptavidin coated microtiter plates previously coated with biotinylated 40 mer oligonucleotides homologous to internal sequences of each endogenous retrovirus/control primer. The probe sequences were as follows:

HML-1

(SEQ ID NO:80)

5′-GGAAAGCTATTAAGCCAGTTAKAGAASAGTTTAAAAAATG;
HML-2/Herv-K

(SEQ ID NO:81)

5′-TAGGTCGAGCTCTTCAACCAGTTAGAGAMAAGTTTTCAGAC;
HervK C4 (HKC4)

(SEQ ID NO:82)

5′-TAGGCAGAACTATCCAGCCTGTTAGAGATCAATTTCCAGAT;
HML-3

(SEQ ID NO:83)

5′-TAGGGCAAGCAATTGAACCTACTCATAMAAAATTTTCACAG;
HML-4

(SEQ ID NO:84)

5′-TGGGGCGTGTGCTTCAACCTGTCAGGGATCAGTTTCCCCGA;
HML-5

(SEQ ID NO:85)

5′-TAAATCAGGCTTTGCTCCCCAGTAGAAAAGAATTTCCTAA;
HML-6

(SEQ ID NO:86)

5′-TAGGACAGGCATTAAAGRAGCCTCGGAATATGTTTCCTACTG;
HTLV-1

(SEQ ID NO:87)

5′-AATGCAGCTGGCCCATATCCTGCAGCCCATTCGGCAAGCTTTCC;
HTLV-2

(SEQ ID NO:88)

5′-ACAACAATTAGCAGCCGTCCTCAACCCCATGAGGAAAATGTTTC;
MMTV

(SEQ ID NO:89)

5′-AAAATTTGTGGACAAAGCTATATTGACTGTAAGGGATAAATACC;
GAPDH

(SEQ ID NO:90)

5′-TTGTCATCAATGGAAATCCCATCACCATCTTCCAGGAGCG;
and
Histone H3

(SEQ ID NO:91)

5′-CAGAAGTCCACTGAACTTCTGATTCGCAAACTTCCCTTCC.

The denatured PCR product was allowed to hybridize for 2 hours at 54° C. Microtiter plate wells were then washed 3 times with TBS and 100 μl of a 1:1000 dilution of anti digoxigenin FAb alkaline phosphatase conjugate (Roche Diagnostics) was added. The plates were then incubated for 60 minutes at room temperature with gentle agitation followed by washing the wells four times with PBS. Then 100 μl of BM chemiluminescence ELISA substrate (Roche Diagnostics) was added and plates were incubated for 10 minutes in the dark. The luminescence was then quantitated using a Tropix Luminometer and accompanying software. Signals from triplicate wells were averaged and subtracted from signals obtained from samples amplified without reverse transcriptase.

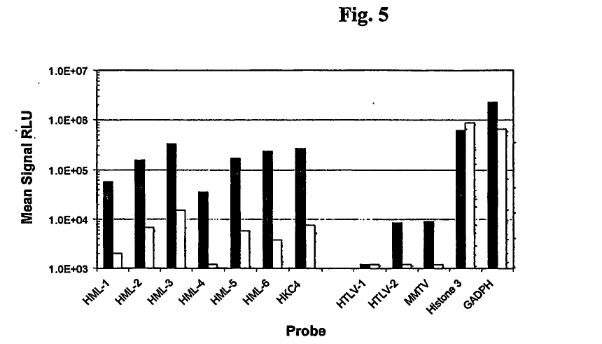

As shown in FIG. 5, strong signals were obtained with the GAPDH and histone H3 primers from both the attached and unattached PBMCs. The unattached cells exhibited only background levels of signal (<10,000 relative light units) with any of the endogenous retroviruses or the negative controls. In contrast, the attached cells exhibited strong signals with the HML-2, 3, 5 and 6 probes and only background levels of signal with the control probes. This indicates that monocyte activation up-regulates expression of HML-2 and most other members of the group of MMTV-related endogenous retroviruses.

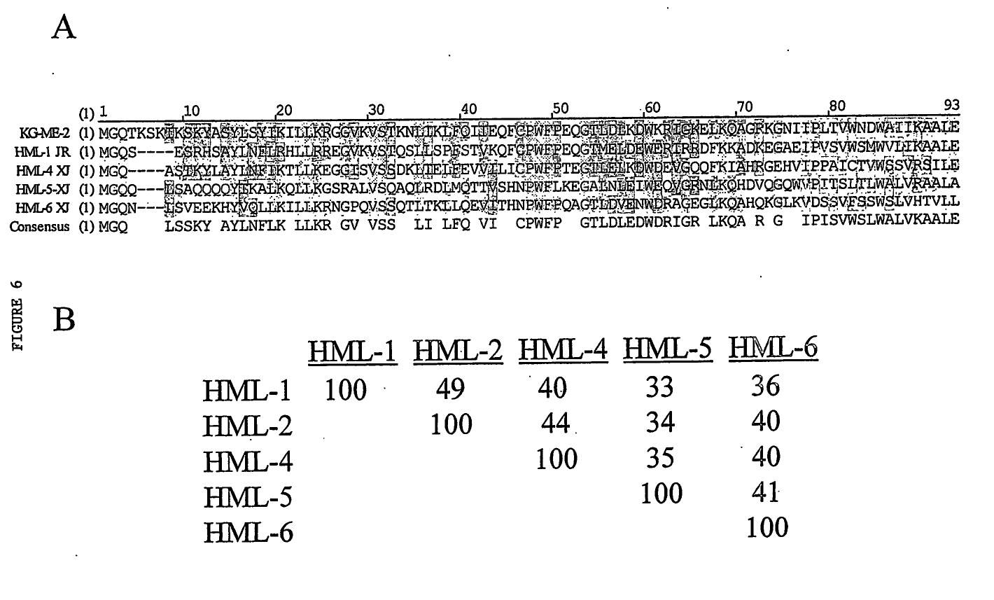

To test whether the observed immunoreactivity to Herv-K/HML-2 antigens in the individuals with ALS might be cross-reactivity of antibodies to an epitope found in other members of the HML family, the 5′ gag sequences of representatives of the HML-1, HML4, HML-5 and HML-6 retroviruses were cloned into a pThioHisA vector and subjected to western blot analysis as described in Example 1. A comparison of the amino acid sequences of the retroviral GAG polypeptides encoded by the clones obtained is presented in FIG. 6A and FIG. 6B. The five polypeptides (including KG-ME-2 from Herv-K/HML-2) are from 33-49% identical to each other, with HML-1 and HML-2 polypeptides sharing an 11 amino acid sequence (QFCPWFPEQGT, SEQ ID NO:92) located in the center of the protein. The corresponding regions of the endogenous viruses HML-1, HML-4, HML-5, and HML-6 are 49%, 44%, 34%, and 40% homologous to the amino acid sequence of KG-ME-2.9, respectively.

Whole cell lysates from bacteria expressing the recombinant HML proteins were analyzed by western blot analysis using sera from individuals with ALS, sera from blood donors (non-ALS individuals) as a control for the ALS sera and, to confirm the presence of a significant amount and the appropriate size of recombinant protein on the blot, a monoclonal antibody to the thioredoxin portion of the fusion protein. Goat anti-human IgG alkaline phosphatase conjugated antibody was used as the secondary antibody to detect serum antibodies bound to the blot. The results of the western blot analysis are shown in Table 7.

TABLE 7
Immunoreactivity of sera with various
MMTV-related GAG polypeptides

| | HML-1 | HML-2 | HML-4 | HML-5 | HML-6 |
|---|---|---|---|---|---|
| ALS | 0/11 | 14/21 | 0/17 | 0/17 | 0/17 |
| | | 67% | | | |
| non-ALS | 0/9 | 6/25 | 0/10 | 0/10 | 0/10 |
| Blood Donors | | 24% | | | |

As shown in Table 7, none of the other four MMTV related 5′ GAG polypeptides reacted with any of the ALS sera tested. Nor was any immunoreactivity seen with the 5′ GAG regions of HML-1, 4, 5 or 6 with sera from non-ALS blood donors. In a direct comparison of 17 sera from ALS patients, 9 of the 17 sera (53%) were immunoreactive with the 5′ GAG region of Herv-K/HML-2 but none of the same 17 sera were immunoreactive with the 5′GAG regions of HML-4, 5 or 6. The immunoreactivity to Herv-K/HML-2 observed in individuals with ALS is specific for Herv-K/HML-2 antigens and does not represent a cross-reaction of antibodies directed to an epitope of a related retrovirus. Thus, ALS patients are specifically reactive with the 5′ gag region of Herv-K/HML-2 and not other endogenous retroviruses.

### Example 5

### Localization of the Reactive Epitope in KG-ME-2

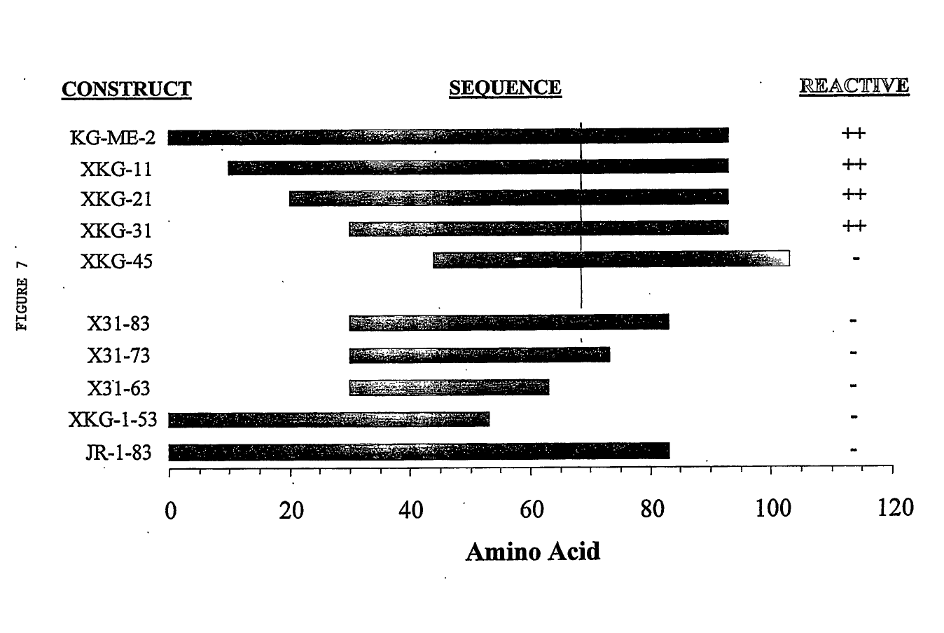

To locate the KG-ME-2 epitope to which the ALS sera was reactive, specific overlapping fragments of the KG-ME-2 polypeptide were generated and subjected to western blot analysis with the sera. The KG-ME-2 polypeptide fragments were generated by amplifying specific portions of the Herv-K/HML-2 gag gene using human genomic DNA or cloned KG-ME-2 DNA as a template. The procedure is described in Example 1 and the primers and templates used in the amplification are listed in Table 1. The amplified DNA was subcloned into the pThioHisA expression vector and the polypeptides were expressed in bacteria as described in Example 1. FIG. 7 contains a diagram indicating amino acid sequences of KG-ME-2 expressed by the various constructs used to localize the epitope within KG-ME-2. To map the amino terminus of the epitope, ten amino acid deletions were introduced into KG-ME-2, starting at its amino terminus and ending at amino acid 30 (clones XKG-11, XKG-21, and XKG-31). Another recombinant protein, XKG-45, was designed that contained the carboxy-terminal 48 amino acids of KG-ME-2, beginning just 5′ to the conserved CPWFP sequence in the middle of the protein. To map the carboxy terminus of the epitope, XKG-31 was used as the starting point and ten amino acid deletions were progressively introduced into the carboxy terminus of XKG-31 to create X31-83, X31-73, and X31-63. XKG-1-53 and JR-1-83 were two other proteins made which retained the original amino terminus of KG-ME-2 but lost 40 and 10 amino acids, respectively, from the carboxy terminus of KG-ME-2.

Whole cell lysates from bacteria expressing recombinant KG-ME-2 polypeptide fragments were analyzed by western blot analysis using ALS sera that was reactive to intact KG-ME-2. Western blots were also performed using a monoclonal antibody to the thioredoxin portion of the fusion protein to confirm the presence of a significant amount and the appropriate size of recombinant protein on the blot. Goat anti-human IgG alkaline phosphatase conjugated antibody was used as the secondary antibody to detect serum antibodies bound to the blot.

Summarized in FIG. 7 are the results of western blot analysis of the KG-ME-2 polypeptide fragments using sera from individuals with ALS. All three proteins, containing amino acids 11 to 93, 21 to 93 and 31 to 93 of KG-ME-2 retained the reactivity observed with full length KG-ME-2. When the protein was further deleted, so that it contained amino acids 45-103 of the Herv-K/HML-2 GAG protein, only 3 sera from individuals with ALS were reactive. Thus, the amino terminus of the epitope begins between amino acids 31 and 45 of KG-ME-2.

None of eleven sera from individuals with ALS reacted with the carboxy terminus deletion polypeptides X31-83 or X31-73. None of 21 sera from individuals with ALS reacted with the protein XKG-1-53 and none of 5 ALS sera reacted with JR-1-83. Thus, the carboxy terminus of the epitope within KG-ME-2 is at or near to amino acid 93 of the Herv-K/HML-2 gag sequence.

Based on this study, the length of the minimal reactive region of KG-ME-2 is about 63 amino acids. Mutational analysis has the potential to determine which of the 63 amino acids are most crucial for proper epitope formation. Additionally, expression of the protein under more native conditions (e.g. in mammalian cell lines) may also provide additional information regarding immunoreactivity of the proteins.

### Example 6

### Correlation of Immunoreactivity to Herv-K/HML-2 Antigen and Clinical Measures

Assays were performed to determine if the observed antibody response to Herv-K/HML-2 correlates with any clinical indicia of ALS, the extent of monocyte activation and/or neuronal disease in the individuals from whom the sera was collected.

Accordingly, antibodies to cell markers and flow cytometry was used to analyze the cell-surface protein expression of circulating T-cells and monocytes from the individuals with ALS. The following panel of fluorophore labeled antibodies directed to the indicated antigens were used in the analyses:
fluorescein-conjugated anti-CD8 (Becton Dickinson);
phycoerythrin-conjugated anti-HLA-DR (Becton Dickinson);
phycoerythrin-conjugated anti-CD38 (Becton Dickinson);
peridinin chlorophyll protein-conjugated anti-CD4 (Becton Dickinson);
peridinin chlorophyll protein-conjugated IgG1 (isotype control, Becton Dickinson);
fluorescein-conjugated anti-CD14 (DAKO Corp.);
phycoerythrin-conjugated anti-CD16 (DAKO Corp.);
fluorescein-conjugated IgG1 (isotype control, DAKO Corp.);
phycoerythrin-conjugated IgG1 (isotype control, DAKO Corp.).

100 μL whole heparinised blood was stained with one or more of the labeled antibodies listed above for 20 minutes at room temperature and protected from light. Red-blood cells were lysed by the addition of 2 ml of FACSLYSE solution (Becton Dickinson, San Jose, Calif.) and a 5 minute incubation. The cell suspensions were centrifuged at 400× g for five minutes. The cell pellets were washed with 1 ml FACSLYSE followed by a wash with 1 ml 0.01 M phosphate-buffered saline (PBS). The cells were fixed with 1 ml of 1% paraformaldehyde in 0.01 M PBS, with 0.1% sodium azide.

Cells were analyzed with a FACSCAN flow cytometer (Becton Dickinson). Antibody staining of the cells was determined by processing at least 10,000 cells per sample through the flow cytometer. Analysis of phenotype was performed by utilizing CELLQUEST software (Becton Dickinson).

The results were then categorized according to whether the individual had an antibody response to KG-ME-2 (IgG and/or IgM) or not. The one individual who was diagnosed with familial ALS was excluded from this analysis. The results of this analysis is shown in Table 7. Statistical analysis using unpaired T-test with Welch's correction for unequal variances (using the program InStat, Graph Pad Software) was used to compare cells from individuals that had an antibody response to KG-ME-2 to cells from individuals that did not have an antibody response to KG-ME-2. In Table 8, ns indicates that the difference in the values is not statistically significant.

TABLE 8
T cells and monocyte correlates of
an antibody response to KG-ME-2

| | anti-KG-ME-2 | | anti-KG-ME-2 | | |
|---|---|---|---|---|---|
| | positive | | negative | | |
| | N | Value | N | Value | Significance |
| T Cells | | | | | |
| % CD4⁺ | 15 | 49.3 | 4 | 43.0 | p = ns |
| Range | | 37.4-58.8 | | 36.2-50.3 | |
| % CD8+ | 15 | 17.9 | 4 | 20.0 | p = ns |
| Range | | 8.4-29.9 | | 13.0-25.7 | |
| CD4⁺/CD8⁺ | 15 | 3.2 | 4 | 2.3 | p = ns |
| Range | | 1.3-6.6 | | 1.4-3.6 | |
| % CD4⁺ CD38⁺ | 15 | 24.4 | 4 | 35.8 | p = ns |
| Range | | 11.6-44.9 | | 18.0-48.7 | |
| % CD8⁺ CD38⁺ | 15 | 15.5 | 4 | 15.4 | p = ns |
| Range | | 5.7-45.4 | | 7.6-22.6 | |
| Macrophage/ | | | | | |
| Monocytes | | | | | |
| % CD14⁺ CD16⁺ | 15 | 42.6 | 4 | 48.4 | p = ns |
| Range | | 22.7-67.4 | | 34.3-67.3 | |
| CD14 side scatter | 15 | 505 | 4 | 618 | p = ns |
| Range | | 323-1023 | | 400-864 | |
| CD14⁺ DR exp. | 15 | 868 | 4 | 904 | p = ns |
| Range | | 395-1243 | | 718-1223 | |

As shown in Table 8, analysis of T-cell surface markers revealed no significant differences in the percentages of CD4⁺ or CD8⁺ T cells between the anti-Herv-K/HML-2 GAG positive and the anti-Herv-K/HML-2 GAG negative individuals. Nor was there any difference between these groups in the number of activated cytotoxic T lymphocytes, as determined by CD38 expression. Although the difference did not reach statistical significance, there was an indication that anti-Herv-K/HML-2 GAG positive individuals with ALS may have lower numbers of activated CD4⁺ T cells (CD4⁺ CD38⁺) than anti-Herv-K/HML-2 GAG negative individuals with ALS. Analysis of circulating monocyte activation also did not reveal any significant differences between anti-Herv-K/HML-2 GAG positive and anti-Herv-K/HML-2 GAG negative individuals with ALS. Levels of CD14⁺/CD16⁺ cells were similar (although very high compared to normal controls) as was the granularity of CD14⁺ cells and expression levels of HLA-DR. Thus the presence oran absence of an antibody response to Herv-K/L-2 GAG was not associated with any significant changes in circulating T cells or monocyte/macrophages.

The length of time with ALS disease and the extent of ALS disease was categorized according to the presence or absence of an antibody response to KG-ME-2. The extent of ALS disease was evaluated according to the ALS Functional Rating Scale. See, for example, The ALS CNTF treatment study (ACTS) phase I-II study group; The Amyotrophic Lateral Sclerosis Functional Rating Scale (1996) Arch. Neurol. 53:141-147. According to this rating scale, a score of 48 indicates no paralysis and a score of 0 indicates complete paralysis. The results of this initial analysis is presented in Table 9.

TABLE 9
Disease correlates of an antibody response to KG-ME-2

| | anti-KG-ME-2 | | anti-KG-ME-2 | | |
|---|---|---|---|---|---|
| | positive | | negative | | |
| ALS Disease | N | Value | N | Value | Significance |
| Months with Disease | 15 | 43.5 | 4 | 15.5 | p = 0.0023 |
| Range | | 4-88 | | 10-19 | |
| Function | 13 | 22.1 | 3 | 32.0 | p = 0.0018 |
| Range | | 8-34 | | 30-34 | |

An shown in Table 9, individuals with an antibody response to Herv-K/HML-2 GAG polypeptide KG-NM-2 had been symptomatic for an average of 43.5 months, a period of time that was, on average, 2.8 times longer than those individuals without antibodies reactive with KG-ME-2; a difference that was highly significant. As also shown in Table 9, antibody positive individuals had significantly lower functional scores. These results indicate that the development of the Herv-K/HML-2 KG-ME-2 antibody response in these individuals was concurrent with the incidence of neurological symptoms.

Using an expanded group of ALS patients, a study of the demographic and clinical characteristics of the patients that had IgG antibodies reactive with the SE-HA antigen (KG-ME-2 amino acids 31-91) to those that were not reactive was conducted. The results of this expanded study are presented in Table 10.

TABLE 10
Clinical and demographic attributes of SE-HA
reactive and negative ALS patients

| Parameter | Positive | Negative |
|---|---|---|
| N (%) | 21 (57%) | 16 (43%) |
| Female (%) | 4 (19%) | 7 (44%) |
| Median age | 60 | 60 |
| (range) | 30-87 | 34-77 |
| Median months since diagnosis | 27.0 | 26.5 |
| (range) | 4-93 | 6-88 |
| Median ALSFRS | 33.5 | 31.0 |
| Range (n) | 13-43 | 8-43 |
| | (n = 18) | (n = 15) |
| Median FVC | 86.0 | 78.5 |
| (range) | 40-123 | 16-100 |
| Used Riluzole (%) | 17 (81%) | 14 (88%) |
| Used Anti Inflammatories (%) | 5 (24%) | 8 (50%) |

As shown in Table 10, individuals whose sera reacted with SE-HA were not distinguishable from non-reactive ALS patients in terms of their age, months since diagnosis, ALSFRS, forced vital capacity (FVC) or frequency of Riluzole therapy (Table 10). ALS patients that had an antibody response to SE-HA were 2 fold less likely to be female (44 vs 19%) and 2 fold less likely to have taken NSAIDs (50 vs 24%), but neither trend reached statistical significance. Including IgM-positive ALS patients with the IgG positive ALS patients did not significantly change the results obtained. Thus, antibody reactivity towards Herv-K SE-HA was not associated with a significantly accelerated or prolonged disease course in this cross-sectional study.

### Example 7

### Expression Herv-K/HML-2 Antigen in Cells

IgG from an ALS sera identified via its immunoreactivity to KG-ME-2 polypeptide was purified on protein-A sepharose according to standard methods and biotinylated using a commercially available kit (Pierce Biotechnology). This biotinylated IgG was then used in concert with anti-human CD14 antibodies to stain monocytes from the blood of several other individuals with ALS. Phycoerythrin-conjugated anti-CD14 was used for the CD14 staining. The stained cells were then analyzed using flow cytometry with streptavidin-labeled FITC using standard methods. Results of this analysis are presented in Table 11.

TABLE 11
PBMC staining

| | Percentage of PBMCs | |
|---|---|---|
| Sample | CD14+/ALS Sera+ | Background |
| ALS-3-5 | 3.3% | |
| ALS-3-6 | 0.6% | |
| ALS-3-7 | 3.2% | |
| ALS-3-8 | 4.5% | <0.1% |
| ALS-3-9 | 2.3% | <0.1% |
| ALS-3-10 | 0.6% | 0.1% |

As shown in Table 11, peripheral blood mononuclear cells O?BMCs) from 4 of 6 individuals tested had between 2% -5% of their monocytes doubly-positive for CD14 and intracellular reactivity with the biotinylated ALS sera. Background staining in these experiments with streptavidin FITC was very low at less than 0.1%. Samples that were negative for ALS sera staining had approximately 0.5% of their monocytes positive. The intracellular reactivity with the biotinylated ALS sera is most likely reactivity to Herv-K/HML-2 GAG polypeptides as supported by results presented herein in other examples. Thus this provides evidence for Herv-K/HML-2 protein expression in a fraction of circulating monocytes in individuals with ALS.

### Example 8

### Expression Herv-K/HML-2 RNA in Cells

Although studies indicate that a low level of Herv-K transcription occurs in PBMCs from healthy individuals (Nedstrand et al. (1993), Medstrand et al. (1992), Brodsky et al. (1993) Blood 81:2369-2374, Depil et al. (2002) Leukemia 16:254-259, Parseval et al. (2003) J Virol. 77:10414-10422), there is no information on levels of Herv-K RNA expression in individuals with neurological disorders. As discussed herein, an immune response to Herv-K in individuals with ALS has the potential to impact the overall level of Herv-K expression in these individuals. Therefore levels of Herv-K genomic RNA expression in circulating PBMC were quantitated in patients with ALS and in patient with Alzheimer's disease (AD), as disease with a neuroinflammatory component.

ALS and AD patient PBMCs were collected as described above and total RNA prepared from aliquots of the cells using the RNAEasy kit according to manufacturer's instructions (Qiagen). The total RNA was then digested with RNAse-free DNAse for 2 hours at 370C to remove any residual DNA contamination. The RNA was then re-purified via phenol-chloroform extraction/ethanol precipitation and resuspended in 250 μl RNAse-free water. Eight microliters of the total RNA was converted to cDNA with random hexamers using a commercially available kit (Roche Applied Sciences, Indianapolis, Ind.) and 10 μl of a 10 fold dilution of the cDNA was subjected to amplification using the Faststart DNA Master SYBR Green kit (Roche Applied Science) with the Search-LC human β-actin amplification kit (Search-LC, Heidelberg, Germany) using the enclosed actin-specific primers. A second 10 μl aliquot was amplified using the Faststart DNA Master SYBR Green kit and primers HML-5A 5′-TTGCCCATG GTT TCC AGA ACA AG (SEQ ID NO:93) and HML-3A 5′-GCT GCT TTA ATA ATG GCC CAA TCA (SEQ ID NO:94). Amplifications were for 50 cycles of 95° C. for 10 seconds, 68° C. for 10 seconds, and 72° C. for 10 seconds on a Light-Cycler controlled with accompanying software (version 3.5, Roche Applied Science). Amplified product was detected via SYBR-Green I fluorescence and the threshold cycle (Ct) at which detectable product was first observed determined for each sample. The Ct for β-actin and Herv-K from each sample was compared to results obtained with 10, 100, 10³, 10⁴ and 10⁵ fold dilutions of cDNA from the cell line Tera-1 that expresses high levels of Herv-K RNA (Boller et al. (1993) Virology 196:349-353). Tera-1 cells were obtained from the American Type Culture Collection (Rockville, Md.) and were cultured in IMDM with 10% fetal calf serum.

Using the standard curve data Herv-K RNA levels were expressed as a fraction of the actin RNA levels according to the formula 10^{((Ctk-Cta×(m}^{ₖ}^{/m}^{ₐ}^{))/m}^{ₖ}⁾ wherein, Ctk refers to the threshold cycles of the sample with the Herv-K primers; Cta refers to the threshold cycle of the sample with the β-actin primers; mₖ refers to the slope of the Tera-1 cDNA standard curve with the Herv-K primers; and mₐ refers to the slope of the Tera-1 cDNA standard curve with the β-actin primers. To control for variations in RNA yield, the level of Herv-K expression in each patient was compared to the level of actin mRNA.

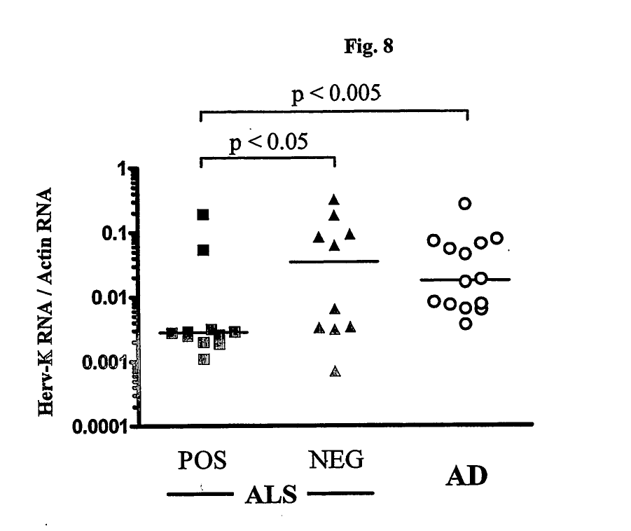

Results from this analysis are presented in FIG. 8. ALS patients who were reactive with Herv-K gag protein had a median Herv-K RNA level that was 0.3% of actin mRNA expression. In contrast, Herv-K antibody negative ALS patients and individuals with AD had median Herv-K RNA levels that were 3.5% (p<0.05, Mann-Whitney test) and 1.8% (p<0.01) of actin, respectively. In this example, the presence of an antibody response to the SE-HA antigen was correlated with a ˜12 fold reduction in the median level of Herv-K RNA in PBMCs in ALS patients. Thus an antibody response to Herv-K is associated with a significant reduction in Herv-K RNA expression. Without wishing to be bound to any particular theory, this result is consistent with an immune response leading to a reduction in Herv-K particle transmission and/or immunological clearance of cells with significant levels of Herv-K protein expression.

What is claimed is:

1. A kit for use in aiding diagnosis of Amyotrophic Lateral Sclerosis (ALS) disease comprising a probe specific for expression of a Herv-K HML-2 gag gene.

2. The kit of claim 1 further comprising instructions for use of the probe in aiding diagnosis of ALS.

3. The kit of claim 1 wherein the probe comprises anti-Herv-K/HML-2 GAG antibodies.

4. The kit of claim 3 wherein the anti-Herv-K/HML-2 GAG antibodies bind a polypeptide comprising amino acids 1 to 93 of SEQ ID NO:2.

5. The kit of claim 3 wherein the anti-Herv-K/HML-2 GAG antibodies bind a polypeptide comprising amino acids 31 to 93 of SEQ ID NO:2.

6. The kit of claim 1 wherein the probe comprises a polypeptide comprising amino acids 1 to 93 of SEQ ID NO:2.

7. The kit of claim 1 wherein the probe comprises a polypeptide comprising amino acids 31 to 93 of SEQ ID NO:2.

8. The kit of claim 1 wherein the probe comprises a polynucleotide comprising the sequence SEQ ID NO:93 and a polynucleotide comprising the sequence SEQ ID NO:94.

9. A kit for use in aiding diagnosis of Amyotrophic Lateral Sclerosis (ALS) disease comprising a probe specific for expression of a Herv-K/HML-2 env gene.

10. The kit of claim 9 further comprising instructions for use of the probe in aiding diagnosis of ALS.

11. The kit of claim 9 wherein the probe comprises anti-Herv-K/HML-2 ENV antibodies.

12. The kit of claim 9 wherein the probe comprises a polypeptide comprising Herv-K/HML-2 ENV or a portion thereof.

13. A method for aiding diagnosis of ALS disease, comprising assaying for expression of Herv-K/HML-2 in a biological sample from an individual.

14. The method of claim 13 wherein the individual is suspected of having ALS.

15. The method of claim 13 wherein the expression of Herv-K/HML-2 is detected by the identification of anti-Herv-K/HML-2 antibodies in the biological sample.

16. The method of claim 15 wherein the anti-Herv-K/HML-2 antibodies are specific for a Herv-K/HML-2 GAG polypeptide or for a Herv-K/HML-2 ENV polypeptide.

17. A method of monitoring ALS therapy in an individual comprising assaying for expression of Herv-K/HML-2 in a biological sample from an individual with ALS disease.

18. The method of claim 17 wherein the expression of Herv-K/HML-2 is detected by the identification of anti-Herv-K/HML-2 antibodies in the biological sample.

19. The method of claim 18 wherein the anti-Herv-K/HML-2 antibodies are specific for a Herv-K/HML-2 GAG polypeptide or for a Herv-K/HML-2 ENV polypeptide.

20. The method of claim 19 wherein the anti-Herv-K/HML-2 GAG antibodies bind a polypeptide comprising amino acids 1 to 93 of SEQ ID NO:2.

21. The method of claim 19 wherein the anti-Herv-K/HML-2 GAG antibodies bind a polypeptide comprising amino acids 31 to 93 of SEQ ID NO:2.

22. The method of claim 17 wherein the expression of Herv-K/HML-2 is detected by the identification of Herv-K/HML-2 RNA in the biological sample.

23. The method of claim 22 wherein the expression of Herv-K/HML-2 is detected by the identification of Herv-K/HML-2 gag or env RNA in the biological sample.

24. The method of claim 23 wherein the Herv-K/HML-2 gag RNA is detected using an oligonucleotides comprising the sequence SEQ ID NO:93 and an oligonucleotides comprising the sequence SEQ ID NO:94 in a polymerase chain reaction technique.

25. A method for classifying ALS disease comprising assaying for expression of Herv-K/HML-2 in a biological sample from an individual with ALS disease.


SEQUENCE LISTING (95 sequences)

SEQ ID NO: 1 - DNA, 278 bp, Herv-K/HML-2
atggggcaaactaaaagtaaaattaaaagtaaatatgcctcttatctcagctatattaaaattcttttaaaaagagggggagttaaagtatctacaaaaaatctaatcaagctatttcaaataatagaacaattttgcccatggtttccagaacaaggaactttagatctaaagattggaaaagaattggtaaggaactaaaacaagcaggtaggaagggtaatatcattccacttacagtatggaatgattgggccattattaaagcagctttagaa

SEQ ID NO: 2 - protein, 93 aa, Herv-K/HML-2
MGQTKSKIKSKYASYLSYIKILLKRGGVKVSTKNLIKLFQIIEQFCPWFPEQGTLDLKDWKRIGKELKQAGRKGNIIPLTVWNDWAIIKAALE

SEQ ID NO: 3 - DNA, 573 bp, Herv-K/HML-2
ccatttcaaacagaagaagatagcatttcagtttctgatgcccctggaagctgtttatagattgtaatgaaaagacaaggaaaaaatcccagaaagaaacggaaagtttacattgcaatatgtagcagagccggtaatggctcagtcaacgcaaaatgttgactataatcaattacagaggtgatatatcctgaaacgttaaaattagaaggaaaaggtccagaattaatggggccatcagagtctaaaccacgaggcacaagtcctcttccagcaggtcaggtgcccgtaagattacaacctcaaacgcaggttaaagaaaataagacccaaccgccagtagcttatcaatactggccgccggctgaacttcagtatcggccacccccagaaagtcagtatggatatccaggaatgcccccagcaccacagggcagggcgccataccctcagccgcccactaggagacttaatcctacggcaccacctagtagacagggtagtgaattacatgaaattattgataaatcaagaaaggaaggagatactgaggcatggcaattcccagtaacg

SEQ ID NO: 4 - protein, 192 aa, Herv-K/HML-2
PFQTEEDSISVSDAPGSCLIDCNEKTRKKSQKETESLHCKYVAEPVMAQSTQNVDYNQLQEVIYPETLKLEGKGPELMGPSESKPRGTSPLPAGQVPVRLQPQTQVKENKTQPPVAYQYWPPAELQYRPPPESQYGYPGMPPAPQGRAPYPQPPTRRLNPTAPPSRQGSELHEIIDKSRKEGDTEAWQFPVT

SEQ ID NO: 5 - DNA, 434 bp, Herv-K/HML-2
aaagctatgcttatggctcaagcaataacaggagttgttttaggaggacaagttagaacatttggaagaaaatgttataattgtggtcaaattggtcacttaaaaaagattgcccagtcttaaacaaacagaatataactattcaagcaactacaacaggtagagagccacctgacttatgtccaagatgtaaaaaaggaaaacattgggctagtcaatgtcattctaaatttgataaaaataggcaaccattgtcaggaaatgagcaaaggggccagcctcaggccccacaacaaactggggcattcccaattcagccatttgttcctcaaagttttcagggacaacaaccccccctgtcccaagtgtttcaaggaataagccagttaccacaatacaacaattgtcccccgccacaagtggcagtgcagcag

SEQ ID NO: 6 - protein, 145 aa, Herv-K/HML-2
KAMLMAQAITGVVLGGQVRTFGRKCYNCGQIGHLKKNCPVLNKQNITIQATTTGREPPDLCPRCKKGKHWASQCHSKFDKNRQPLSGNEQRGQPQAPQQTGAFPIQPFVPQSFQGQQPPLSQVFQGISQLPQYNNCPPPQVAVQQ

SEQ ID NO: 7 - DNA, 708 bp, Herv-K/HML-2
ttagaaccgatgccacctggagaaggagcccaagagggagagcctcccacagttgaggccagatacaagtctttttcgataaaaatgctaaaagatatgaaagagggagtaaaacagtatggacccaactccccttatatgaggacattattagattccattgctcatggacatagactcattccttatgattgggagattctggcaaaatcgtctctctcaccctctcaatttttacaatttaagacttggtggattgatggggtacaagaacaggtccgaagaaatagggctgccaatcctccagttaacatagatgcagatcaactattaggaataggtcaaaattggagtactattagtcaacaagcattaatgcaaaatgaggccattgagcaagttagagctatctgccttagagcctgggaaaaaatccaagacccaggaagtacctgcccctcatttaatacagtaagacaaggttcaaaagagccctatcctgattttgtggcaaggctccaagatgttgctcaaaagtcaattgccgatgaaaaagcccataaggtcatagtggagttgatggcatatgaaaacgccaatcctgagtgtcaatcagccattaagccattaaaaggaaaggttcctgcaggatcagatgtaatctcagaatatgtaaaagcctgtgatggaatcggaggagctatgcat

SEQ ID NO: 8 - protein, 236 aa, Herv-K/HML-2
LEPMPPGEGAQEGEPPTVEARYKSFSIKMLKDMKEGVKQYGPNSPYMRTLLDSIAHGHRLIPYDWEILAKSSLSPSQFLQFKTWWIDGVQEQVRRNRAANPPVNIDADQLLGIGQNWSTISQQALMQNEAIEQVRAICLRAWEKIQDPGSTCPSFNTVRQGSKEPYPDFVARLQDVAQKSIADEKAHKVIVELMAYENANPECQSAIKPLKGKVPAGSDVISEYVKACDGIGGAMH

SEQ ID NO: 9 - DNA, 716 bp, Herv-K/HML-2
tggttggtagaagtacctactgtcagtcccatcagtagattcacttatcccatggtaagcgggatgtcactcaggccacgggtaaattattcacaagacttttcttatcaaagatcatttaaatttagacctaaagggaacccttgccccaaggaaattcccaaagaatcaaaaaatacagaagttttagtttgggaagaatgtgtggccaatagtgcggtgatattacaaaacaatgaatttggaactattatagattgggcacctcgaggtcaattctaccacaattgctcaggacaaactcagtcgtgtccaagtgcacaagtgagtccagctgttgatagcgacttaacagaaagtttagacaaacataagcataaaaaattgcagtctttctacccttgggaatggggagaaaaaggaatctctaccccaagaccaaaaataataagtcctgtttctggtcctgaacatccagaattatggaggcttactgtggcctcacaccacattagaatttggtctggaaatcaaactttagaaacaagagatcgtaagccattttatactgtcgacctaaattccagtctaacagttcctttacaaagttgcataaagcccccttatatgctagttgtaggaaatatagttattaaaccagactcccagactataacctgtgaaaattgtagattgcttacttgcattgattcatc

SEQ ID NO: 10 - protein, 239 aa, Herv-K/HML-2
WLVEVPTVSPISRFTYPMVSGMSLRPRVNYSQDFSYQRSFKFRPKGNPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTVPLQSCIKPPYMLVVGNIVIKPDSQTITCENCRLLTCIDSS

SEQ ID NO: 11 - DNA, 345 bp, Herv-K/HML-2
tggttggtagaagtacctactgtcagtcccatcagtagattcacttatcacatggtaagcgggatgtcgctcaggccacaggtaaattatttacaagacttttcttatcaaagatcattaaaatttagacttaaagggaaaccttgccccaaggaaattcccaaagaatcaaaaaatacagaagttttagtttgggaagaatgtgtggccaatagtgcggtgatattacaaaacaatgaattcggaactattatagattgggcacctcgaggtcaattcgatcacaattgctcaggacaaactcagttgtgtccaagtgcacaagtgagtccagctgttgatagc

SEQ ID NO: 12 - protein, 115 aa, Herv-K/HML-2
WLVEVPTVSPISRFTYHMVSGMSLRPQVNYLQDFSYQRSLKFRLKGKPCPKEIPKESKNTEVLVWEECVANSAVILQNNEFGTIIDWAPRGQFDHNCSGQTQLCPSAQVSPAVDS

SEQ ID NO: 13 - DNA, 419 bp, Herv-K/HML-2
cactcttctgttcagtcagtaaactttgttaatgatgggcaaaaaaattctacaagattgtggaattcacaatctggtattgatcaaaaattggcaaatcaaattaatgatcttagacaaactgtcatttggatgggagacagactcatgagcttagaacatcgtttccagttacagtgtgactggaatacatcagatttttgtattacaccccaaatttataatgagtctgagcatcactgggacatggttagacgtcatctacagggaagagaagataatctcactttagacatttccaagttaaaagaacaaattttcaaaacatcaaaagcccatttaaatttggtgccaggaactgaggcaattgcaggagttgctgatggcctcgcaaatcttaaccctgtcacttgggttaa

SEQ ID NO: 14 - protein, 140 aa, Herv-K/HML-2
HSSVQSVNFVNDGQKNSTRLWNSQSGIDQKLANQINDLRQTVIWMGDRLMSLEHRFQLQCDWNTSDFCITPQIYNESEHHWDMVRRHLQGREDNLTLDISKLKEQIFKTSKAHLNLVPGTEAIAGVADGLANLNPVTWVK

SEQ ID NO: 15 - DNA, 35 bp, HML-2
gcggaattcctcgagatggggcaaactaaaagtaa

SEQ ID NO: 16 - DNA, 40 bp, HML-2
ctgtcgacgcggccgcgttctaaagctgctttaataatgg

SEQ ID NO: 17 - DNA, 36 bp, HML-2
gcggaattcctcgagccatttcaaacagaagaagat

SEQ ID NO: 18 - DNA, 38 bp, HML-2
ctgtcgacgcggccgcacgttactgggaattgccatgc

SEQ ID NO: 19 - DNA, 36 bp, HML-2
gcggaattcctcgagttagaaccgatgccacctgga

SEQ ID NO: 20 - DNA, 38 bp, HML-2
ctgtcgacgcggccgctatgcatagctcctccgattcc

SEQ ID NO: 21 - DNA, 36 bp, HML-2
gaggaattcctcgagaaagctatgcttatggctcaa

SEQ ID NO: 22 - DNA, 38 bp, HML-2
ctgtcgacgcggccgcactgctgcactgccacttgtgg

SEQ ID NO: 23 - DNA, 34 bp, HML-2
gcggaattcctcgagtggttggtagaagtaccta

SEQ ID NO: 24 - DNA, 38 bp, HML-2
ctgtcgacgcggccgcgatgaatcaatgcaagtaagca

SEQ ID NO: 25 - DNA, 34 bp, HML-2; identical to SEQ ID NO: 23

SEQ ID NO: 26 - DNA, 38 bp, HML-2
ctgtcgacgcggccgccgctatcaacagctggactcac

SEQ ID NO: 27 - DNA, 33 bp, HML-2
gcggaattcctcgagcactcttctgttcagtca

SEQ ID NO: 28 - DNA, 33 bp, HML-2
ctgtcgacgcggccgcttaacccaagtgacagg

SEQ ID NO: 29 - DNA, 36 bp, Herv-W
gcggaattcctcgagatggccctcccttatcatatt

SEQ ID NO: 30 - DNA, 39 bp, Herv-W
tcggtcgactgcggccgcagggggaggcatatccaacag

SEQ ID NO: 31 - DNA, 33 bp, Herv-W
gcgcaattcctcgagggagttcaagatcaggca

SEQ ID NO: 32 - DNA, 40 bp, Herv-W
tcggtcgactgcggccgctctcttttgttgcggggcttag

SEQ ID NO: 33 - DNA, 35 bp, Herv-W
gcgcaattcctcgagttctactacaaactatctca

SEQ ID NO: 34 - DNA, 40 bp, Herv-W
tcggtcgactgcggccgccattggctgaggaggccccagg

SEQ ID NO: 35 - DNA, 35 bp, Herv-W
gcggaattcctcgagatgttctccaccctccccaa

SEQ ID NO: 36 - DNA, 38 bp, Herv-W
ctgtcgacgcggccgcggcataattggggaatattggc

SEQ ID NO: 37 - DNA, 36 bp, Herv-W
gcggaattcctcgagcaaaaggagatagacaaaagg

SEQ ID NO: 38 - DNA, 35 bp, Herv-W
ctgtcgacgcggccgcctgtggggaatgtttctct

SEQ ID NO: 39 - DNA, 33 bp, Herv-W
gcggaattcctcgagttatgccctacaggaagc

SEQ ID NO: 40 - DNA, 35 bp, Herv-W
ctgtcgacgcggccgcgtcctaacccttgtaaaac

SEQ ID NO: 41 - DNA, 36 bp, Herv-W
gcggaattcctcgagtttggcgatctctggtatctc

SEQ ID NO: 42 - DNA, 35 bp, Herv-W
ctgtcgacgcggccgcccagaaaggcagtaggatt

SEQ ID NO: 43 - DNA, 33 bp, Herv-W
gcggaattcctcgagatgatgtccaccataaca

SEQ ID NO: 44 - DNA, 35 bp, Herv-W
ctgtcgacgcggccgcctgcagctgactgagtgat

SEQ ID NO: 45 - DNA, 33 bp, HML-1
cgcgaattcctcgagatgggacaaagtgaaagc

SEQ ID NO: 46 - DNA, 35 bp, HML-1
ctgtcgacgcggccgcgctcaagagctgcctttat

SEQ ID NO: 47 - DNA, 33 bp, HML-4
gcggaattcctcgagatgggacaagccagtaca

SEQ ID NO: 48 - DNA, 35 bp, HML-4
ctgtcgacgcggccgcattctaagatggagcgaac

SEQ ID NO: 49 - DNA, 33 bp, HML-5
gcggaattcctcgagatgggacaacagttatca

SEQ ID NO: 50 - DNA, 35 bp, HML-5
ctgtcgacgcggccgcgggccagagctgccctaac

SEQ ID NO: 51 - DNA, 33 bp, HML-6
gcggaattcctcgagatgtgcagttgcttagag

SEQ ID NO: 52 - DNA, 35 bp, HML-6
ctgtcgacgcggccgcgcagaagtacagtatgaac

SEQ ID NO: 53 - DNA, 31 bp, HML-2
gcggaattcaaatatgcctcttatctcagct

SEQ ID NO: 54 - DNA, 38 bp, HML-2
ctgtcgacgcggccgcgttctaaagctgctttaataat

SEQ ID NO: 55 - DNA, 30 bp, HML-2
gcggaattcattcttttaaaaagaggggga

SEQ ID NO: 56 - DNA, 38 bp, HML-2; identical to SEQ ID NO: 54

SEQ ID NO: 57 - DNA, 30 bp, HML-2
gcggaattctctacaaaaaatctaatcaag

SEQ ID NO: 58 - DNA, 38 bp, HML-2; identical to SEQ ID NO: 54

SEQ ID NO: 59 - DNA, 27 bp, HML-2
gcggaattcwtttgcccwtggttycca

SEQ ID NO: 60 - DNA, 38 bp, HML-2
ctgtcgacgcggccgcctgaaacactatcttcttctgt

SEQ ID NO: 61 - DNA, 30 bp, HML-2; identical to SEQ ID NO: 57

SEQ ID NO: 62 - DNA, 38 bp, HML-2; identical to SEQ ID NO: 54

SEQ ID NO: 63 - DNA, 30 bp, HML-2; identical to SEQ ID NO: 57

SEQ ID NO: 64 - DNA, 39 bp, HML-2
ctgtcgacgcggccgcccttcctacctgcttgttttagt

SEQ ID NO: 65 - DNA, 30 bp, HML-2; identical to SEQ ID NO: 57

SEQ ID NO: 66 - DNA, 39 bp, HML-2
ctgtcgacgcggccgccaattcttttccaatcttttaga

SEQ ID NO: 67 - DNA, 35 bp, HML-2; identical to SEQ ID NO: 15

SEQ ID NO: 68 - DNA, 38 bp, HML-2
ctgtcgacgcggccgcttccttgttctggaaaccatgg

SEQ ID NO: 69 - DNA, 35 bp, HML-2; identical to SEQ ID NO: 15

SEQ ID NO: 70 - DNA, 33 bp, HML-2
gcggccgcattccatactgtaagtggaatgata

SEQ ID NO: 71 - DNA, 28 bp, HML-2
tcgacccatcaccatcaccatcattgca

SEQ ID NO: 72 - DNA, 20 bp, HML-2
atgatggtgatggtgatggg

SEQ ID NO: 73 - protein, 9 aa, Artificial Sequence
  Synthetic construct
YPYDVPDYA

SEQ ID NO: 74 - DNA, 29 bp, Artificial Sequence
  Primer
gaaggatcctggamdgtnytdcchcargg

SEQ ID NO: 75 - DNA, 26 bp, Artificial Sequence
  Primer
gtcggatccnwdatrtcatcmatrta

SEQ ID NO: 76 - DNA, 21 bp, Artificial Sequence
  Primer
cggagtcaacggatttggtcg

SEQ ID NO: 77 - DNA, 24 bp, Artificial Sequence
  Primer
agccttctccatggtggtgaagac

SEQ ID NO: 78 - DNA, 23 bp, Artificial Sequence
  Primer
ccctctactggaggggtgaagaa

SEQ ID NO: 79 - DNA, 23 bp, Artificial Sequence
  Primer
cttgcctcctgcaaagcaccgat

SEQ ID NO: 80 - DNA, 40 bp, HML-1
  Primer
ggaaagctattaagccagttakagaasagtttaaaaaatg

SEQ ID NO: 81 - DNA, 41 bp, Herv-K/HML-2
  Primer
taggtcgagctcttcaaccagttagagamaagttttcagac

SEQ ID NO: 82 - DNA, 41 bp, Herv-K C4
  Primer
taggcagaactatccagcctgttagagatcaatttccagat

SEQ ID NO: 83 - DNA, 41 bp, HML-3
  Primer
tagggcaagcaattgaacctactcatamaaaattttcacag

SEQ ID NO: 84 - DNA, 41 bp, HML-4
  Primer
tggggcgtgtgcttcaacctgtcagggatcagtttccccga

SEQ ID NO: 85 - DNA, 40 bp, HML-5
  Primer
taaatcaggctttgctccccagtagaaaagaatttcctaa

SEQ ID NO: 86 - DNA, 42 bp, HML-6
  Primer
taggacaggcattaaagragcctcggaatatgtttcctactg

SEQ ID NO: 87 - DNA, 44 bp, HTLV-1
  Primer
aatgcagctggcccatatcctgcagcccattcggcaagctttcc

SEQ ID NO: 88 - DNA, 44 bp, HTLV-2
  Primer
acaacaattagcagccgtcctcaaccccatgaggaaaatgtttc

SEQ ID NO: 89 - DNA, 44 bp, MMTV
  Primer
aaaatttgtggacaaagctatattgactgtaagggataaatacc

SEQ ID NO: 90 - DNA, 40 bp, Homo Sapiens
  Primer
ttgtcatcaatggaaatcccatcaccatcttccaggagcg

SEQ ID NO: 91 - DNA, 40 bp, Homo Sapiens
  Primer
cagaagtccactgaacttctgattcgcaaacttcccttcc

SEQ ID NO: 92 - protein, 11 aa, HML-1 and HML-2
  Primer
QFCPWFPEQGT

SEQ ID NO: 93 - DNA, 23 bp, HML-5
  Primer
ttgcccatggtttccagaacaag

SEQ ID NO: 94 - DNA, 24 bp, HML-3
  Primer
gctgctttaataatggcccaatca

SEQ ID NO: 95 - protein, 5 aa, Artificial Sequence
  Synthetic Construct
HHHHH